(19) United States
(12) Patent Application Publication
(10) Pub. No.: US 2021/0236485 A1
(43) Pub. Date: Aug. 5, 2021

(54) REPEATED ADMINISTRATION OF DIHYDROERGOTAMINE FOR TREATMENT OF FREQUENT MIGRAINE HEADACHES

(71) Applicant: Impel Neuropharma, Inc., Seattle, WA (US)

(72) Inventors: John D. Hoekman, Seattle, WA (US); Kelsey H. Satterly, Seattle, WA (US); Stephen B. Shrewsbury, Fallbrook, CA (US); Scott Youmans, Bothell, WA (US); Christopher Fuller, Seattle, WA (US)

(21) Appl. No.: 17/148,154

(22) Filed: Jan. 13, 2021

Related U.S. Application Data
(60) Provisional application No. 62/961,076, filed on Jan. 14, 2020.

(51) Int. Cl. A61K 31/4985; A61K 9/00; A61K 31/522; A61K 47/26; A61P 25/06

(52) U.S. Cl. CPC A61K 31/4985; A61K 9/008; A61P 25/06; A61K 47/26; A61K 31/522

(57) ABSTRACT

Methods are provided for reducing the frequency of migraine attacks in a subject who has frequent migraine headaches with or without aura. The methods comprise intranasally administering to the subject a pharmaceutical composition comprising dihydroergotamine (DHE) or salt thereof on a repeat dose schedule, wherein each intranasal administration is delivered by a manually actuated, propellant-driven, metered-dose administration device, and wherein the schedule is a chronic intermittent schedule in which each of the repeated administrations is performed while the subject is experiencing a migraine headache.

### 1. CROSS-REFERENCE TO RELATED APPLICATIONS

This application claims priority to U.S. provisional application No. 62/961,076, filed Jan. 14, 2020, the disclosure of which is herein by reference in its entirety.

### 2. BACKGROUND

Migraine is a common and disabling neurologic disorder experienced by more than 80 million people in the United States and European Union. Most people who are prone to migraines get a painful attack once or twice a month. But some migraineurs get headaches more frequently. These frequent and severe attacks impair quality of life.

Migraine treatments can be categorized as acute treatments, intended to curtail or reduce the intensity of ongoing migraine attacks, or chronic treatments, intended to reduce the frequency of migraine attacks. Prior to the development of CGRP antagonists, most research and development efforts focused on the development of acute treatments for migraine. For example, dihydroergotamine (DHE), a semisynthetic derivative of the ergot alkaloid ergotamine, has been approved for over 70 years for the acute treatment of migraines. The exact mechanism of action of DHE is not known, but DHE is known to act as a serotonin receptor agonist, cause vasoconstriction of intracranial blood vessels, and interact centrally with dopamine and adrenergic receptors.

The oral bioavailability of DHE is poor, and DHE is commonly administered parenterally as the mesylate salt by subcutaneous, intramuscular or intravenous injection, and where approved, by nasal spray. Because migraine headaches are episodic and occur unpredictably, administration by nasal spray is far more convenient for treatment of migraine than is administration by injection. However, the previously approved nasal spray drug-device combination product provides only 32% of the bioavailability of the intravenous injection, and variable efficacy (among other factors) has led to its withdrawal from the market in the EU and other countries, although it remains available in the United States.

There is, therefore, a need for improved agents and methods for treating acute migraine attacks, including improved methods of administering DHE, and a need for agents and methods that are capable of reducing the frequency of migraine attacks.

### 3. SUMMARY

The present disclosure provides a method of reducing the frequency of migraine attacks in a subject who has frequent migraine headaches with or without aura. The method is based on the discovery from a Phase III clinical trial that intranasally administering a pharmaceutical composition comprising dihydroergotamine (DHE) mesylate on a repeat dose schedule, wherein each intranasal administration is delivered by a manually actuated, propellant-driven, metered-dose administration device, and wherein the schedule is a chronic intermittent schedule in which each of the repeated administrations is performed while the subject is experiencing a migraine headache, is effective both to treat acute symptoms, and over repeated dosing, is also effective in reducing the frequency of migraine attacks. As described in detail in Example 3, administration provided acute relief pain—including pain relief at 2 hours, and increased pain freedom and relief of the most bothersome symptom at 2 hours—and upon repeated PRN dosing, reduced migraine attack frequency. The acute relief, coupled with reduced frequency of migraine attack, led to improvements in MIDAS and HIT-6 scores, scores that measure the impact of migraine headaches on quality of life.

In the Phase 3 trial, doses were administered as a single divided 1.45 mg intranasal dose of dihydroergotamine (DHE) mesylate using our Precision Olfactory Delivery (POD®) Device. This drug-device combination product, “INP104”, is a manually actuated, propellant-driven, intranasal administration device that can reproducibly deliver metered doses of liquid pharmaceutical compositions beyond the nasal valve to more distal regions of the nasal cavity. When tested in an earlier Phase I clinical trial described in detail in Example 2, INP104 provided 4-fold higher mean maximal plasma concentration, nearly 3-fold higher mean systemic drug exposure, and reached maximal DHE plasma concentration faster than a 2.0 mg dose of DHE mesylate administered intranasally using Migranal® Nasal Spray according to the US FDA approved product label. The higher maximal plasma concentration and systemic drug exposure were achieved with a lower administered dose of the identical formulation of DHE mesylate, 1.45 mg for INP104 versus 2.0 mg for Migranal®, and without requiring a 15-minute wait between administration of divided sub-doses, as required for Migranal®. In addition, systemic delivery of DHE was more consistent with INP104 than with Migranal®, with lower coefficient of variation (CV %) in DHE AUC_{0-inf} and Cₘₐₓ observed across subjects.

Accordingly, in a first aspect, methods are provided for reducing the frequency of migraine attacks in a subject who has frequent migraine headaches with or without aura, comprising: administering a pharmaceutical composition comprising dihydroergotamine (DHE) or salt thereof via a respiratory track of the subject on a repeat dose schedule, wherein the schedule is a chronic intermittent schedule in which each of the repeated administrations is performed while the subject is experiencing a migraine headache.

In some embodiments, the step of administering is performed by intranasal administration. In some embodiments, the intranasal administration is performed with a manually actuated, propellant driven, metered dose administration device. In some embodiments, the step of administering is performed by pulmonary administration. In some embodiments, the repeat dose schedule comprises administration of at least a first dose and a second dose of the pharmaceutical composition.

In some embodiments, the pharmaceutical composition is a liquid pharmaceutical composition. In some embodiments, each of the doses is administered as two divided subdoses. In some embodiments, the divided subdoses are administered into separate nostrils. In some embodiments, the divided subdoses are administered within no more than 1 minute. In some embodiments, the divided subdoses are administered within no more than 45 seconds. In some embodiments, the divided doses are administered within no more than 30 seconds.

In some embodiments, prior to a first manual actuation, the pharmaceutical composition and propellant are not in contact within the device. In some embodiments, the pharmaceutical composition is contained in a vial and the propellant is contained in a canister, wherein the canister is a pressurized canister. In some embodiments, between successive manual actuations, the pharmaceutical composition in the vial and propellant in the canister are not in contact within the device. In some embodiments, each manual actuation brings a metered volume of the pharmaceutical composition and a separately metered volume of propellant into contact within a dose chamber of the device. In some embodiments, contact of propellant with the pharmaceutical composition within the dose chamber of the device creates a spray of the pharmaceutical composition as the formulation is expelled through a nozzle of the device. In some embodiments, the nozzle has plurality of lumens, and the spray is ejected simultaneously through a plurality of nozzle lumens. In some embodiments, the propellant is a hydrofluoroalkane propellant. In some embodiments, the propellant is hydrofluoroalkane-134a.

In some embodiments, prior to a first actuation, the vial is nonintegral to the device and is configured to be attached thereto. In some embodiments, the vial is configured to be threadably attachable to the device. In some embodiments, each of the doses of the pharmaceutical composition comprises no more than 2.0 mg DHE or salt thereof. In some embodiments, each of the doses of the pharmaceutical composition comprises less than 2.0 mg DHE or salt thereof. In some embodiments, each of the doses of the pharmaceutical composition comprises 1.2-1.8 mg DHE or salt thereof. In some embodiments, each of the doses of the pharmaceutical composition comprises 1.4-1.6 mg DHE or salt thereof. In some embodiments, each of the doses of the pharmaceutical composition comprises about 1.45 mg DHE or salt thereof.

In some embodiments, the liquid composition is administered as two divided subdoses in two sprays, wherein each of the two divided subdoses is 140-250 μL. In some embodiments, each of the two divided doses is 175-225 μL. In some embodiments, each of the two divided doses is about 200 μL. In some embodiments, the liquid composition comprises a salt of DHE. In some embodiments, the liquid composition comprises DHE mesylate. In some embodiments, the liquid composition comprises DHE mesylate at a concentration of 2.5-7.5 mg/ml. In some embodiments, the liquid composition comprises DHE mesylate at a concentration of 3.5-6.5 mg/ml. In some embodiments, the liquid composition comprises DHE mesylate at a concentration of about 4.0 mg/ml.

In some embodiments, the liquid composition further comprises caffeine. In some embodiments, the liquid composition comprises caffeine at a concentration of 10 mg/ml.

In some embodiments, the liquid composition further comprises dextrose. In some embodiments, the liquid composition comprises dextrose at a concentration of 50 mg/ml.

In some embodiments, the pharmaceutical composition comprises 4.0 mg/ml DHE mesylate, 10.0 mg/ml caffeine, and 50 mg/ml dextrose.

In some embodiments, the pharmaceutical composition is a dry powder pharmaceutical composition.

In some embodiments, the intranasal administration is delivered by an intranasal dispenser device. In some embodiments, the device comprises an air source that is adapted to be engaged by a user to force air from an air source through a valve assembly into a reservoir and out of a nozzle. In some embodiments, the device is operated by applying compressive force to a pump. In some embodiments, the pump comprises a manual air pump. In some embodiments, the dry powder pharmaceutical composition comprises DHE or salt thereof and at least one member selected from the group consisting of a thickening agent, a carrier, a pH adjusting agent, and a sugar alcohol.

In some embodiments, the dry powder pharmaceutical composition comprises the thickening agent, wherein the thickening agent is selected from the group consisting of hydroxypropyl methylcellulose (HPMC), hydroxypropyl cellulose, methyl cellulose, carboxymethylcellulose calcium, sodium carboxymethylcellulose, sodium alginate, xanthan gum, acacia, guar gum, locust bean gum, gum tragacanth, starch, carbopols, methylcellulose, and polyvinylpyrrolidone. In some embodiments, the thickening agent is HPMC.

In some embodiments, the dry pharmaceutical composition comprises the carrier, wherein the carrier is selected from microcrystalline cellulose, ethyl cellulose, cellulose acetate, cellulose acetate butyrate, cellulose acetate propionate, cellulose acetate phthalate, hydroxypropylmethylcellulose phthalate, starch, chitosan, and βcyclodextrin. In some embodiments, the carrier is microcrystalline cellulose.

In some embodiments, the dry pharmaceutical composition comprises the sugar alcohol, wherein the sugar alcohol is selected from the group consisting of mannitol, glycerol, galactitol, fucitol, inositol, volemitol, maltotriitol, maltoetetraitol, polyglycitol, erythritol, threitol, ribitol, arabitol, xylitol, allitol, dulcitol, glucitol, sorbitol, altritol, iditol, maltitol, lactitol, and isomalt. In some embodiments, the sugar alcohol is mannitol.

In some embodiments, the dry pharmaceutical composition further comprises a fluidizing agent, wherein the fluidizing agent comprises a calcium phosphate. In some embodiments, the fluidizing agent comprises tribasic calcium phosphate.

In some embodiments, the dry pharmaceutical composition comprises the salt of DHE, wherein the salt is DHE mesylate. In some embodiments, the dry powder pharmaceutical composition comprises DHE mesylate at a concentration of 0.01-0.2 mg/mg. In some embodiments, the dry powder pharmaceutical composition comprises DHE mesylate at a concentration of 0.01-0.1 mg/mg. In some embodiments, the dry powder pharmaceutical composition comprises DHE mesylate at a concentration of 0.016-0.07 mg/mg. In some embodiments, the dry powder pharmaceutical composition comprises DHE mesylate at a concentration of 0.02-0.07 mg/mg.

In some embodiments, the dry powder pharmaceutical composition comprises DHE mesylate, a first microcrystalline cellulose (MCC-1), a second microcrystalline cellulose (MCC-2), and tribasic calcium phosphate (TCP). In some embodiments, the dry powder pharmaceutical composition comprises DHE mesylate and a first microcrystalline cellulose (MCC-1). In some embodiments, the dry powder pharmaceutical composition comprises DHE mesylate, MCC-1, HPMC, Mannitol, MCC-2 and TCP. In some embodiments, the dry powder pharmaceutical composition comprises DHE mesylate, MCC-1, HPMC and Mannitol. In some embodiments, the dry powder pharmaceutical composition comprises DHE mesylate, MCC-1, HPMC, Mannitol, a pH adjuster, MCC-2 and TCP. In some embodiments, the dry powder pharmaceutical composition comprises DHE mesylate, MCC-1, HPMC, Mannitol and a pH adjuster. In some embodiments, the dry powder pharmaceutical composition comprises DHE mesylate, MCC-1, HPMC and Mannitol.

In some embodiments, the pharmaceutical composition comprises particles having an average diameter from 10-300 μm. In some embodiments, the average diameter is from 15-200 μm. In some embodiments, the average diameter is from 20-100 μm. In some embodiments, the particles are spray dried, freeze-dried, or melt-extruded.

In some embodiments, the dry pharmaceutical composition is formulated in a unit dose. In some embodiments, the unit dose comprises 3-6 mg of DHE mesylate. In some embodiments, the unit dose comprises 3.9 mg of DHE mesylate. In some embodiments, the unit dose comprises 5.2 mg of DHE mesylate. In some embodiments, each dose of the dry pharmaceutical composition administered comprises 3-6 mg of DHE or a salt thereof. In some embodiments, each dose comprises 3.9 mg of DHE or a salt thereof. In some embodiments, each dose comprises 5.2 mg of DHE or a salt thereof.

In some embodiments, following administration of the first dose, the mean peak plasma DHE concentration (Cmax) is at least 750 pg/ml. In some embodiments, following administration of the first dose, the DHE Cmax is at least 1000 pg/ml. In some embodiments, following administration of the first dose, the DHE Cmax is at least 1200 pg/ml. In some embodiments, following intranasal administration of the first dose, the DHE Cmax is at least 2000 pg/ml. In some embodiments, following administration of the first dose, the mean time to Cmax (Tmax) of DHE is less than 45 minutes. In some embodiments, following intranasal administration of the first dose, the DHE Tmax is no more than 30 minutes. In some embodiments, following intranasal administration of the first dose, the DHE Tmax is about 30 minutes. In some embodiments, following administration of the first dose, the mean plasma AUC0-inf of DHE is at least 2500 pg*hr/ml. In some embodiments, following administration of the first dose, the mean plasma AUC0-inf of DHE is at least 3000 pg*hr/ml. In some embodiments, following administration of the first dose, the mean plasma AUC0-inf of DHE is at least 4000 pg*hr/ml. In some embodiments, following administration of the first dose, the mean plasma AUC0-inf of DHE is at least 5000 pg*hr/ml. In some embodiments, following administration of the first dose, the mean plasma AUC0-inf of DHE is at least 6000 pg*hr/ml. In some embodiments, following administration of the first dose, the mean plasma AUC0-inf of DHE is at least 10000 pg*hr/ml. In some embodiments, following administration of the first dose, the mean peak plasma concentration (Cmax) of 8″ OH-DHE is at least 50 pg/ml.

In some embodiments, following administration of the first dose, the mean Cmax of 8′ OH-DHE is at least 55 pg/ml. In some embodiments, following administration of the first dose, the mean plasma AUC0-inf of 8′ OH-DHE is at least 1000 pg*hr/ml.

In some embodiments, the subject has at least three migraine attacks in the 4-week period immediately preceding administration of the first dose. In some embodiments, the subject has at least four migraine attacks in the 4-week period immediately preceding administration of the first dose. In some embodiments, the subject has fewer than 3 migraine headaches during the 4-week period immediately following administration of the second dose.

In some embodiments, the subject has fewer than 2 migraine headaches during the 4-week period immediately following administration of the second dose. In some embodiments, the subject has no migraine headaches during the 4-week period immediately following administration of the second dose. In some embodiments, the subject has fewer than 6 migraine headaches during the 8-week period immediately following administration of the second dose. In some embodiments, the subject has fewer than 4 migraine headaches during the 8-week period immediately following administration of the second dose. In some embodiments, the subject has fewer than 2 migraine headaches during the 8-week period immediately following administration of the second dose. In some embodiments, the subject has fewer than 12 migraine headaches in the 12-week period immediately following administration of the second dose. In some embodiments, the subject has fewer than 6 migraine headaches during the 12-week period immediately following administration of the second dose. In some embodiments, the subject has fewer than 3 migraine headaches during the 12-week period immediately following the repeated administrations. In some embodiments, the subject has fewer than 18 migraine headaches during the 24-week period immediately following the repeated administrations. In some embodiments, the subject has fewer than 12 migraine headaches during the 24-week period immediately following administration of the second dose. In some embodiments, the subject has fewer than 4 migraine headaches during the 24-week period immediately following administration of the second dose.

In some embodiments, the frequency of migraine headaches is reduced by at least 50% during the 4-week period immediately following administration of the second dose as compared to the frequency of migraine headaches during the 4 week-period immediately preceding administration of the first dose. In some embodiments, the frequency of migraine headaches is reduced by at least 60% during the 4-week period immediately following administration of the second dose as compared to the frequency of migraine headaches during the 4 week-period immediately preceding administration of the first dose. In some embodiments, the frequency of migraine headaches is reduced by at least 75% during the 4-week period immediately following administration of the second dose as compared to the frequency of migraine headaches during the 4 week-period immediately preceding administration of the first dose. In some embodiments, administration of the first dose of the repeated administrations of the pharmaceutical composition reduce one or more symptoms selected from pain, nausea, phonophobia, and photophobia. In some embodiments, reduction of the one or more symptoms occurs at 2 hours post administration. In some embodiments, the subject has migraine that does not respond to triptan drugs. In some embodiments, each of the repeated administrations is performed by a self-administration.

In some embodiments, the repeat dose schedule lasts at least one month. In some embodiments, the repeat dose schedule lasts at least two months. In some embodiments, the repeat dose schedule lasts at least three months. In some embodiments, the repeat dose schedule lasts at least four months. In some embodiments, the repeat dose schedule lasts at least five months. In some embodiments, the repeat dose schedule lasts at least six months.

In some embodiments, the repeat dose schedule lasts 5 to 8 weeks. In some embodiments, the repeat dose schedule lasts 9 to 12 weeks. In some embodiments, the repeat dose schedule lasts 13 to 16 weeks. In some embodiments, the repeat dose schedule lasts 17 to 20 weeks. In some embodiments, the repeat dose schedule lasts 21 to 24 weeks.

In some embodiments, the repeat dose schedule lasts at least 5 weeks. In some embodiments, the repeat dose schedule lasts at least 9 weeks. In some embodiments, the repeat dose schedule lasts at least 13 weeks. In some embodiments, the repeat dose schedule lasts at least 17 weeks. In some embodiments, the repeat dose schedule lasts at least 21 weeks.

In another aspect, a pharmaceutical composition comprising dihydroergotamine (DHE) or salt thereof is provided for us in a method of reducing the frequency of migraine attacks in a subject who has frequent migraine headaches with or without aura, wherein the method comprises intranasally administering to the subject the pharmaceutical composition on a repeat dose schedule, wherein each intranasal administration is delivered by a manually actuated, propellant-driven, metered-dose administration device, and wherein the schedule is a chronic intermittent schedule in which each of the repeated administrations is performed while the subject is experiencing a migraine headache.

In yet another aspect, kits are provided for treating frequent migraine headache with or without aura. The kits comprise a vial, within which is sealably contained at least one effective dose of a liquid pharmaceutical composition comprising dihydroergotamine (DHE) or salt thereof, and a device, wherein the vial is configured to be attachable to the device, and wherein the device, upon attachment of the vial, is a manually actuated, metered-dose, propellant-driven intranasal administration device capable of providing, after intranasal administration of a dose of liquid pharmaceutical composition, (a) a mean peak plasma DHE concentration (Cₘₐₓ) of at least 750 pg/ml, (b) with a mean time to Cₘₐₓ (Tₘₐₓ) of DHE of less than 45 minutes, and (c) a mean plasma AUC_{0-inf} of DHE of at least 2000 pg*hr/ml.

In some embodiments, the device within the kit comprises a canister, wherein the canister is a pressurized canister containing propellant.

In various kit embodiments, the vial contains no more than 2 ml of liquid pharmaceutical composition. In some embodiments, the vial contains approximately 1 ml of liquid pharmaceutical composition.

In some embodiments, the pressurized canister contains an amount of propellant sufficient to administer no more than 1 dose of liquid pharmaceutical composition.

Other features and advantages of the present disclosure will become apparent from the following detailed description, including the drawings. It should be understood, however, that the detailed description and the specific examples are provided for illustration only, because various changes and modifications within the spirit and scope of the invention will become apparent to those skilled in the art from the detailed description.

### 4. BRIEF DESCRIPTION OF THE DRAWINGS

FIG. 1 shows a cross section of an embodiment of a handheld, manually actuated, metered-dose, propellant-driven intranasal administration device useful for precision olfactory delivery of dihydroergotamine (DHE).

FIGS. 2A, 2B and 2C show a cross section of the nasal delivery device of FIG. 1 in the stages of rest and actuation. FIG. 2A shows the nasal delivery device at rest with FIG. 2B showing the actuation of the pump and FIG. 2C showing actuation of the propellant valve.

FIG. 3 shows a cross section of another implementation of the nasal delivery device.

FIG. 4 shows a cross section of the diffuser as seated within the device.

FIG. 5A shows an exploded view of the dose chamber and the Y-junction unassembled.

FIG. 5B shows an exploded view of the dose chamber and Y-junction in cooperation.

FIG. 6 shows arrows representing both dose and propellant flow.

FIG. 7 shows the actuator grip and conical spring arrangement.

FIG. 8 shows a cross section of the optional nose cone and a side elevation of the optional nose cone.

FIGS. 9A and 9B illustrate the device used in the phase I clinical trial described in Example 2, with further description of the numbered parts set forth in Table 1.

FIGS. 10A and 10B plot plasma concentrations of DHE versus time as measured in the phase I comparative bioavailability clinical trial described in Example 2, with FIG. 10A plotting data from 0 to 8 hours and FIG. 10B plotting data from 0 to 24 hours.

FIGS. 11A and 11B plot plasma concentrations of the 8′-OH-DHE metabolite of DHE versus time as measured in the phase 1 comparative bioavailability clinical trial described in Example 2, with FIG. 11A plotting data from 0 to 8 hours and FIG. 11B plotting data from 0 to 24 hours.

FIG. 12A shows a cross section of an alternate implementation of the nasal delivery device.

FIG. 12B shows a zoomed-in view of the cross section of FIG. 12A.

FIG. 13A shows a cross section of the diffuser as seated within the device, according to an additional embodiment.

FIG. 13B shows an exploded view of the nozzle and the Y-junction, according to an additional embodiment.

FIG. 14 illustrates the nose cone, according to an additional embodiment.

FIG. 15 illustrates the schematic design of the Phase III clinical trial study described in Example 3.

FIG. 16 shows screening period treatments for 302 subjects who participated in the Phase III clinical trial. 1,396 migraines of the 302 subjects were treated with Best Usual Care (e.g., triptans, acetaminophen, NSAID, opioids, barbiturate, combination analgesic) during 4-week screening period.

FIGS. 17A and 17B provide percentages (%) of patients demonstrating pain freedom at 2 hours after treatment with Lasmiditan (placebo, 100 mg, or 200 mg), Rimegepant (placebo or 75 mg), Ubrogepant (placebo, 25 mg, or 50 mg), MAP0004-DHE (placebo or 1.0 mg) or INP104. Data for lasmiditan, rimegepant, ubrogepant and MAP0004-DHE are historical.

FIG. 18 provide percentages (%) of patients demonstrating freedom from most bothersome symptom at 2 hours after treatment with Lasmiditan (100 mg or 200 mg) (historical), Rimegepant (75 mg) (historical), Ubrogepant (50 mg) (historical), Best Usual Care or INP104.

FIG. 19A provides percentages of patients having pain relief after administration of a first dose of INP104, from 15 mins to 120 mins following administration. FIG. 19B provides a table with data from earlier studies, summarizing percentages of patients reporting pain relief at 2 hours after treatment with Lasmiditan (200 mg), Rimegepant (75%), Ubrogepant (100 mg), MAP0004, or Migranal.

FIG. 20 plots migraine attack frequency over time during the 4-week screening period and the 24-week treatment period of the Phase III clinical trial described in Example 3.

FIG. 21 plots percentages of subjects who provided a positive answer (neutral, agree, or strongly agree) to each question related to their treatment experience with INP104 in the Phase III clinical trial described in Example 3.

FIG. 22 plots mean percentage of migraine attacks that were pain free 2 hours after administration of INP104 in the 24-week treatment period, demonstrating maintenance of the acute relief effect through repeat administrations (absence of tachyphylaxis).

FIG. 23 plots mean percentage of migraine attacks that were pain free 2 hours after administration of INP104 in the 52-week treatment period, demonstrating maintenance of the acute relief effect through repeat administrations.

FIG. 24 plots mean percentage of migraine attacks free of the most bothersome symptom (MBS) at 2 hours after INP104 administration in 24-week treatment period, demonstrating maintenance of the acute relief effect through repeat administrations.

FIG. 25 plots mean percentage of migraine attacks free of the most bothersome symptom (MBS) at 2 hours after INP104 administration in 52-week treatment period, demonstrating maintenance of the acute relief effect through repeated administrations.

FIG. 26 plots mean percentage of migraine attacks with pain relapse at 24 hours after INP104 administration in 24-week treatment period.

### 5. DETAILED DESCRIPTION

### 5.1. Definitions

Unless defined otherwise, all technical and scientific terms used herein have the meaning commonly understood by a person skilled in the art to which this invention belongs.

As used herein, the terms “migraine”, “migraine without aura”, and “migraine with aura” are as defined in The International Classification of Headache Disorders, 3^{rd} edition, Cephalalgia 38(1):1-211 (2018), incorporated in its entirety by reference herein.

The term “frequent migraine headaches” or “frequent migraines” as used herein refer to a frequency of at least two migraine attacks per month for a 6-month period.

The term “dose” as used herein refers to a quantity of a medicine or drug taken or recommended to be taken at a particular time. The term “subdose” as used herein refers to a portion of the dose, which is less than an entirety of the dose. In typical embodiments, a dose is divided into two or more subdoses.

The term “initial administration period” as used herein refers to a period inclusive of and immediately following the first dose of a treatment agent.

### 5.2. Other Interpretational Conventions

Ranges: throughout this disclosure, various aspects of the invention are presented in a range format. Ranges include the recited endpoints. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Unless specifically stated or apparent from context, as used herein the term “or” is understood to be inclusive.

Unless specifically stated or apparent from context, as used herein, the terms “a”, “an”, and “the” are understood to be singular or plural. That is, the articles “a” and “an” are used herein to refer to one or to more than one (i.e., to at least one) of the grammatical object of the article. By way of example, “an element” means one element or more than one element.

In this disclosure, “comprises,” “comprising,” “containing,” “having,” “includes,” “including,” and linguistic variants thereof have the meaning ascribed to them in U.S. Patent law, permitting the presence of additional components beyond those explicitly recited.

Unless specifically stated or otherwise apparent from context, as used herein the term “about” is understood as within a range of normal tolerance in the art, for example within 2 standard deviations of the mean and is meant to encompass variations of ±20% or ±10%, more preferably ±5%, even more preferably ±1%, and still more preferably ±0.1% from the stated value.

### 5.3. Summary of Experimental Observations

We designed a manually actuated, propellant-driven, intranasal administration device that can reproducibly deliver metered doses of liquid pharmaceutical compositions beyond the nasal valve to more distal regions of the nasal cavity. We tested the device in a Phase I clinical trial designed to compare the bioavailability of (i) dihydroergotamine (DHE) mesylate administered as a single divided 1.45 mg intranasal dose using this Precision Olfactory Delivery (POD™) Device (“INP104”); (ii) a 2.0 mg dose of DHE mesylate administered intranasally using Migranal® Nasal Spray according to the US FDA approved product label; and (iii) a 1.0 mg intravenous injection of DHE mesylate for injection (D.H.E. 45®), in healthy adult subjects.

As described in detail in Example 2, INP104 provided nearly 3-fold higher mean systemic drug exposure, nearly 4-fold higher mean maximal plasma concentration, and reached maximal DHE plasma concentration faster than Migranal®. The higher systemic drug exposure and higher maximal plasma concentration were achieved with a lower administered dose of the identical formulation of DHE mesylate, 1.45 mg for INP104 versus 2.0 mg for Migranal®, and without requiring a 15-minute wait between administration of divided sub-doses, as required for Migranal®.

In addition, systemic delivery of DHE was more consistent with INP104 than with Migranal®, with lower variation observed across subjects for both AUC_{0-inf} and Cₘₐₓ parameters.

Although bolus intravenous administration of 1 mg DHE mesylate provided greater than 10-fold higher Cₘₐₓ than 1.45 mg DHE mesylate administered intranasally by INP104, the high Cₘₐₓ achieved with intravenous administration is known to be correlated with adverse events (“AE”s), specifically nausea, and IV DHE mesylate is most commonly administered with an anti-emetic. Within 20-30 minutes following administration, DHE plasma concentrations achieved through INP104 intranasal administration were essentially indistinguishable from concentrations achieved by intravenous administration. Thus, despite a greater than 10-fold higher Cₘₐₓ, bolus intravenous administration of 1 mg DHE mesylate provided less than 2-fold greater systemic drug delivery, measured as AUC_{0-inf}, as compared to INP104 intranasal delivery.

The 8′OH-DHE metabolite of DHE is known to be active, and to contribute to the long-lasting effect of DHE on migraine. We found that intranasal administration of 1.45 mg DHE mesylate by INP104 provides equivalent systemic exposure to the active metabolite of DHE as bolus intravenous administration of 1.0 mg DHE mesylate. In contrast, the 8′-OH DHE metabolite could be detected in only a minority of subjects administered Migranal®.

Safety and therapeutic effects of INP104 were further tested in a Phase III interventional, open-label, single-group assignment, safety, tolerability and exploratory efficacy study (NCT03557333, “STOP-301”). Subjects with at least 2 migraine attacks per month self-administered INP104 (1.45 mg DHE in a divided dose, one actuation per nostril) intranasally when they experience a recognizable migraine. They used no more than 2 doses of INP104 within a 24-hour period, 3 doses in a 7-day period, and 12 doses per 4-week period.

As described in detail in Example 3, upon receiving a first dose of INP104, 38% of subjects reported being free of pain at 2 hours, and 53% of subjects reported being free of the most bothersome symptom at 2 hours. These results exceed those reported in the literature for Lasmiditan, Rimegepant, Ubrogepant, and MAP20004-DHE. Additionally, pain relief effects of INP104 appeared faster than has been reported with other treatment methods. At 15 mins after INP104 administration, 16.8% of subjects reported pain relief, whereas only 9% and 8% of subjects are reported in the literature to have had pain relief 15 mins after administration of MAP0004 and Rimegepant, respectively.

Surprisingly, administration of a plurality of doses of INP104 on a repeat dose schedule also reduced migraine frequency. The reduction of migraine frequency was sustained throughout the 24-week treatment period. The 147 patients who completed the 24-week treatment period showed about 44% reduction in migraine frequency in the 24-week treatment period. A 45 patient subgroup of those who completed the 24-week treatment period, those who had fewer than 12 migraine attacks treated over the 24-week treatment period, showed about 76% reduction in the migraine frequency. Further, the number of reported headaches overall and the number of migraine attacks experienced during each postbaseline 4-week interval decreased substantially, especially in the first 12 weeks, compared with the baseline measure of the total headaches treated with standard-of-care acute medication.

### 5.4. Methods of Treating Frequent Migraine with or without Aura

Accordingly, in a first aspect, methods are provided for treating a subject with frequent migraine headaches with or without aura by administering to the respiratory system of a subject a plurality of doses of a pharmaceutical composition comprising dihydroergotamine (DHE) or salt thereof on a repeat dose schedule, to achieve sustained reduced frequency of migraine headaches over a period of time.

The methods comprise administering to the respiratory system of a subject a plurality of doses of a pharmaceutical composition comprising dihydroergotamine (DHE) or salt thereof on a repeat dose schedule, wherein the schedule comprises at least (i) the administration of a first dose of the pharmaceutical composition and (ii) the subsequent administration of a second dose of the pharmaceutical composition within a 28-day initial administration period, and wherein the plurality of doses are sufficient to reduce the frequency of migraine headaches during the 4-week period immediately following the 28-day initial administration period as compared to the frequency of migraine headaches during the 4 week-period immediately preceding the 28-day initial administration period.

### 5.4.1. Administration Methods

The repeat dose schedule comprises the administration of two or more doses of the pharmaceutical composition within the 28-day initial administration period. In some embodiments, the schedule comprises the administration of 2, 3, 4, 5, 6, 7, 8, 9, 10, 11, or 12 doses of the pharmaceutical composition within the 28-day initial administration period. In some embodiments, the schedule comprises administration of a first dose and administration of a second dose of the pharmaceutical composition within the 28-day initial administration period. In some embodiments, the schedule further comprises administration of a third dose of the pharmaceutical composition within the 28-day initial administration period. In some embodiments, the schedule further comprises administration of a fourth dose of the pharmaceutical composition within the 28-day initial administration period. In some embodiments, the schedule further comprises administration of a fifth dose of the pharmaceutical composition within the 28-day initial administration period. In some embodiments, the schedule further comprises administration of a sixth dose of the pharmaceutical composition within the 28-day initial administration period. In some embodiments, the schedule further comprises administration of a seventh dose of the pharmaceutical composition within the 28-day initial administration period. In some embodiments, the schedule further comprises administration of an eighth dose of the pharmaceutical composition within the 28-day initial administration period. In some embodiments, the schedule further comprises administration of a ninth dose of the pharmaceutical composition within the 28-day initial administration period. In some embodiments, the schedule further comprises administration of a tenth dose of the pharmaceutical composition within the 28-day initial administration period. In some embodiments, the schedule further comprises administration of an eleventh dose of the pharmaceutical composition within the 28-day initial administration period. In some embodiments, the schedule further comprises administration of a twelfth dose of the pharmaceutical composition within the 28-day initial administration period.

In some embodiments, the schedule further comprises at least one additional administration following the 28-day initial administration period. In some embodiments, the schedule further comprises 2, 3, 4, 5, 6, 7, 8, 9, 10 or more additional administrations following the 28-day initial administration period.

In some embodiments, the repeat dose schedule lasts at least one month. In some embodiments, the repeat dose schedule lasts at least two months. In some embodiments, the repeat dose schedule lasts at least three months. In some embodiments, the repeat dose schedule lasts at least four months. In some embodiments, the repeat dose schedule lasts at least five months. In some embodiments, the repeat dose schedule lasts at least six months. In some embodiments, the repeat dose schedule lasts at least seven months. In some embodiments, the repeat dose schedule lasts at least eight months. In some embodiments, the repeat dose schedule lasts at least nine months. In some embodiments, the repeat dose schedule lasts at least ten months. In some embodiments, the repeat dose schedule lasts at least eleven months. In some embodiments, the repeat dose schedule lasts at least twelve months.

In some embodiments, the repeat dose schedule lasts 5-8 weeks. In some embodiments, the repeat dose schedule lasts 9-12 weeks. In some embodiments, the repeat dose schedule lasts 13-16 weeks. In some embodiments, the repeat dose schedule lasts 17-20 weeks. In some embodiments, the repeat dose schedule lasts 21-24 weeks. In some embodiments, the repeat dose schedule lasts 25-28 weeks. In some embodiments, the repeat dose schedule lasts 29-32 weeks. In some embodiments, the repeat dose schedule lasts 33-36 weeks. In some embodiments, the repeat dose schedule lasts 37-40 weeks. In some embodiments, the repeat dose schedule lasts 41-44 weeks.

In some embodiments, the repeat dose schedule lasts at least 5 weeks. In some embodiments, the repeat dose schedule lasts at least 9 weeks. In some embodiments, the repeat dose schedule lasts at least 13 weeks. In some embodiments, the repeat dose schedule lasts at least 17 weeks. In some embodiments, the repeat dose schedule lasts at least 21 weeks. In some embodiments, the repeat dose schedule lasts at least 25 weeks. In some embodiments, the repeat dose schedule lasts at least 29 weeks. In some embodiments, the repeat dose schedule lasts at least 33 weeks. In some embodiments, the repeat dose schedule lasts at least 37 weeks. In some embodiments, the repeat dose schedule lasts at least 41 weeks.

In some embodiments, the schedule comprises administration of no more than 20, 18, 16, 14, 12, 10, 8, 6, 4, or 2 doses of the pharmaceutical composition within any 28-day period. In some embodiments, the schedule comprises administration of no more than 12 doses of the pharmaceutical composition within any 28-day period.

In some embodiments, the schedule comprises administration of no more than 10, 9, 8, 7, 6, 5, 4, 3, 2, or 1 dose of the pharmaceutical composition within any 7-day period. In some embodiments, the schedule comprises administration of no more than 3 doses of the pharmaceutical composition within any 7-day period.

In some embodiments, the schedule comprises administration of no more than 2 doses of the pharmaceutical composition within any 24-hour period.

In some embodiments, the schedule is a chronic intermittent schedule in which each administration is performed while the subject is experiencing a migraine headache. In some embodiments, each administration is performed within 240, 120, 90, 60, 45, 30, 15 or 10 minutes when the subject starts experiencing a migraine headache.

In some embodiments, the schedule is a fixed schedule in which administrations are performed at prespecified intervals. In some embodiments, the schedule is 3, 2, or 1 administration per week. In some embodiments, the schedule is one administration per week. In some embodiments, the schedule is two administrations per week.

In various embodiments, each of the repeated administrations is performed within 5 minutes, 10 minutes, 15 minutes, 30 minutes, 45 minutes, 60 minutes, or 120 minutes of onset of at least one prodromal symptom. In various embodiments, each of the repeated administrations is performed within 5 minutes, 10 minutes, 15 minutes, 30 minutes, 45 minutes, 60 minutes, or 120 minutes of onset of at least one acute symptom.

In some embodiments, the subject uses concomitant medications for prevention or treatment of migraine headache. Exemplary concomitant medication includes, but not limited to, acetaminophen, aspirin, and ibuprofen or other nonsteroidal anti-inflammatory drugs (NSAIDs). In typical embodiments, concomitant medications are administered only at 2 hours after administration of the composition using methods described herein. In some embodiments, non-ergot or non-triptan analgesics are used for subjects who still have headache pain.

In some embodiments, a single dose is administered as two or more divided subdoses. In some embodiments, the divided subdoses are administered into separate nostrils. In some embodiments, the divided subdoses are administered within no more than 1 minute. In some embodiments, the divided subdoses are administered within no more than 45 seconds or 30 seconds.

In some embodiments, the subjects are additionally treated with other methods known to be effective in treating migraines. The methods include, but not limited to, Botox injection, treatment with antibodies designed to target calcium gene-related peptide (CGRP) inhibitors and the CGRP receptor, CGRP receptor antagonists (e.g., gepants), medications used to treat high blood pressure such as beta-blockers (e.g., propranolol, timolol, metoprolol) and calcium channel blockers (e.g., verapamil), antidepressants such as amitriptyline and nortriptyline, antiseizure medications such as gabapentin, topiramate and valproic acid, and nontraditional supplement treatments for migraine prevention such as PA-free butterbur, coenzyme-Q10 and feverfew. In some embodiments, the subjects are additionally treated with Lasmiditan, Rimegepant, Ubrogepant, or MAP20004-DHE.

In some embodiments, the subject performs the administration (self-administration). In some embodiments, the administration is performed by another individual, such as a parent, guardian, caregiver, or medical professional.

### 5.4.2. Patients with Frequent Migraine

Patients who can be treated with the methods provided herein have frequent migraine headaches or a migraine symptom, which includes, but not limited to, pain, nausea, photophobia, nausea, phonophobia, foggy thinking, vomiting, visual changes, other pain, smell, dizziness, or touch sensitivity. Patients are subjects who have migraine headaches or a migraine symptom at least twice per month.

The patient with frequent migraine headaches can have two migraine attacks per month on average before treatment with a method provided herein. In some embodiments, the patient with frequent migraine headaches has three migraine attacks per month on average before treatment with a method provided herein. In some embodiments, the patient with frequent migraine headaches has four, five, six, seven, eight, nine, ten, or more migraine attacks per month on average before treatment with a method provided herein. In some embodiments, the patient with frequent migraine headaches has at least two migraine attacks per month on average before treatment with a method provided herein. In some embodiments, the patient with frequent migraine headaches has at least three migraine attacks per month on average before treatment with a method provided herein. In some embodiments, the patient with frequent migraine headaches has at least four, five, six, seven, eight, nine, ten, or more migraine attacks per month on average before treatment with a method provided herein. In some embodiments, the patient with frequent migraine headaches has less than ten migraine attacks per month on average before treatment with a method provided herein. In some embodiments, the patient with frequent migraine headaches has less than nine migraine attacks per month on average before treatment with a method provided herein. In some embodiments, the patient with frequent migraine headaches has less than eight, seven, six, five, four, or three migraine attacks per month on average before treatment with a method provided herein.

The methods described herein can be used to treat frequent migraine headaches in a subject when the subject is experiencing a recognizable migraine headache. In some embodiments, the treatment can achieve sustained reduced frequency of migraine headaches over a period of time.

In some embodiments, the subjects have been diagnosed with migraine per International Headache Society (IHS) criteria. In some embodiments, the subjects have been diagnosed with migraine per other medical criteria. The subject can have migraine headaches with or without aura. In some embodiments, the subject does not have chronic migraine, medication overuse headache, or other chronic headache syndromes as per International Classification of Headache Disorders version 3 beta (ICHD3b) criteria. In some embodiments, the subject does not have trigeminal autonomic cephalalgias (including cluster headache, hemicrania syndromes and short-lasting, unilateral, neuralgiform headache attacks with conjunctival injection and treating), migraine aura without headache, hemiplegic migraine or migraine with brainstem aura (previously referred to as basilar migraines), chronic migraines, medication overuse headache or other chronic headache syndromes, as per by International Classification of Headache Disorders version 3 beta criteria. In some embodiments, the subject does not have positive test for human immunodeficiency virus, hepatitis B surface antigen, or hepatitis C antibodies. In some embodiments, the subject does not have ischemic heart disease or clinical symptoms or findings consistent with coronary artery vasospasm, including Prinzmetal's variant angina. In some embodiments, the subject does not have significant risk factors for coronary artery disease or medical history of diabetes or smoking, known peripheral arterial disease, Raynaud's phenomenon, sepsis or vascular surgery (within 3 months prior to study start), or severely impaired hepatic or renal function. In some embodiments, the subject does not have significant nasal congestion, physical blockage in either nostril, significantly deviated nasal septum, septal perforation, or any pre-existing nasal mucosal abnormality on endoscopy scoring 1 or more (except score 1 allowed for mucosal edema). In some embodiments, the subject has not previously shown hypersensitivity to ergot alkaloids or any of the ingredients in the drug product. In some embodiments, the subject has not previously failed to respond to intravenous DHE for treatment of migraine. In some embodiments, the subject has not used for more than 12 days per month triptan or ergot-based medication in the 2 months prior to treatment with a method provided herein.

Migraine headache can be defined by ICHD3b criteria. Typically, a migraine is a type of primary headache that some people get repeatedly over time. Migraines can occur with symptoms such as nausea, vomiting, or sensitivity to light. For some migraine patients, a throbbing pain is felt only on one side of the head. Migraines treated with a method provided herein can be migraine “with aura” or “without aura.” An aura is a group of neurological symptoms, usually vision disturbances that serve as warning sign.

In some embodiments, the subject has frequent migraine headache with aura. In some embodiments, the subject has frequent migraine headache without aura. In some embodiments, the subject has had onset of at least one prodromal symptom of migraine. In a variety of embodiments, migraine to be treated is menstrual-associated migraine. In some embodiments, migraine to be treated has proven resistant to triptans.

In various embodiments, the subject has had onset of at least one prodromal symptom of migraine, without onset of headache pain. In certain embodiments, the subject has had onset of at least one prodromal symptom selected from neck stiffness, facial paresthesia, photosensitivity, acoustic sensitivity, and visual aura.

In various embodiments, the subject has had onset of at least one symptom associated with acute migraine. In certain embodiments, the subject has had onset of at least one symptom selected from visual aura; headache pain, including dull, throbbing, or pulsing pain; photosensitivity; acoustic sensitivity; nausea; vomiting. Visual aura and headache pain may be unilateral or bilateral, focal or diffuse.

In various embodiments, the methods are used for acute treatment of cluster headaches rather than migraine.

In various embodiments, the subject has had migraine headaches at least twice, three times, four times, five times, six times, or more a month prior to the repeated administration.

In certain embodiments, the subject has triptan-resistant migraine. In some embodiments, the subject does not respond to treatment with triptan. Exemplary triptans includes, but is not limited to, Almotriptan (Axert), Eletriptan (Relpax), Frovatriptan (Frova), Naratriptan (Amerge), Rizatriptan (Maxalt), Sumatriptan (Imitrex), and Zolmitriptan (Zomig). In some embodiments, the subject does not respond to combinatory therapy of migraine using to triptan. In some embodiments, the subject does not respond to Sumatriptan in combination with naproxen sodium (Treximet). In some embodiments, the subject does not use triptan or ergot-based medication or medication strongly or moderately affecting CYP3A4 Cytochrome P450 metabolic pathway.

In some embodiments, the subject is receiving regular migraine preventive treatment for at least 30 days preceding the 28-day initial administration period. In some embodiments, the subject is receiving one or more concomitant medications including, but is not limited to, beta-blocker and tricyclic antidepressant, unless they are contraindicated for concomitant use with an ergot derivative. In some embodiments, the subject does not receive regular migraine preventive treatment for at least 30 days preceding the 28-day initial administration period.

In some embodiments, the subject has at least one, at least two, at least three, at least four, at least five, or at least six migraine attacks as defined by ICHD3b criteria in the 4-week period immediately preceding the 28-day initial administration period. In some embodiments, the subject has at least two migraine attacks as defined by ICHD3b criteria in the 4-week period immediately preceding the 28-day initial administration period. In some embodiments, the subject has at least four migraine attacks as defined by ICHD3b criteria in the 4-week period immediately preceding the 28-day initial administration period.

In some embodiments, the subject has at least one, at least two, at least three, at least four, at least five, or at least six migraine attacks as defined by ICHD3b criteria in the 6-month period immediately preceding the 28-day initial administration period. In some embodiments, the subject has at least two migraine attacks as defined by ICHD3b criteria in the 6-month period immediately preceding the 28-day initial administration period. In some embodiments, the subject has at least four migraine attacks as defined by ICHD3b criteria in the 6-month period immediately preceding the 28-day initial administration period.

In some embodiments, the subject experiences a variety of most bothersome symptoms during the migraine attack. The subject can experience photophobia, nausea, phonophobia, foggy thinking, vomiting, visual changes, other pain, smell, dizziness, or touch sensitivity.

In some embodiments, the subject is a human. In some embodiments, the subject is a non-human animal. In some embodiments, the subject is an adult. In some embodiments, the subject is a male. In some embodiments, the subject is a female.

### 5.4.3. Migraine Reduction

In various embodiments, the repeated administrations of the pharmaceutical composition reduce one or more symptoms selected from pain, nausea, phonophobia, and photophobia. In some embodiments, the repeated administrations of the pharmaceutical composition reduce the frequency or severity of migraine as measured by pain, nausea, phonophobia, and photophobia. In some embodiments, the repeated administrations of the pharmaceutical composition reduce incidence of pain relapse within 2, 4, 6, 12, 24, or 48 hours after the administration.

In some embodiments, the subject has a reduction in migraine headache frequency during the 4-week period immediately following the 28-day initial administration period as compared to the frequency of migraine headaches during the 4 week-period immediately preceding the 28-day initial administration period. In some embodiments, the subject has a reduction in migraine headache frequency during the 8-week period immediately following the 28-day initial administration period as compared to the frequency of migraine headaches during the 4 week-period immediately preceding the 28-day initial administration period. In some embodiments, the subject has a reduction in migraine headache frequency during the 12-week period immediately following the 28-day initial administration period as compared to the frequency of migraine headaches during the 4 week-period immediately preceding the 28-day initial administration period. In some embodiments, the subject has a reduction in migraine headache frequency during the 16-week period immediately following the 28-day initial administration period as compared to the frequency of migraine headaches during the 4 week-period immediately preceding the 28-day initial administration period. In some embodiments, the subject has a reduction in migraine headache frequency during the 20-week period immediately following the 28-day initial administration period as compared to the frequency of migraine headaches during the 4 week-period immediately preceding the 28-day initial administration period.

In some embodiments, a reduction in migraine headache frequency lasts at least 4 weeks after the 28-day initial administration period. In some embodiments, a reduction in migraine headache frequency lasts at least 8 weeks after the 28-day initial administration period. In some embodiments, a reduction in migraine headache frequency lasts at least 12 weeks after the 28-day initial administration period. In some embodiments, a reduction in migraine headache frequency lasts at least 16 weeks after the 28-day initial administration period. In some embodiments, a reduction in migraine headache frequency lasts at least 20 weeks after the 28-day initial administration period. In some embodiments, a reduction in migraine headache frequency lasts at least 24 weeks after the 28-day initial administration period. In some embodiments, a reduction in migraine headache frequency lasts at least 28 weeks after the 28-day initial administration period.

In some embodiments, the frequency of migraine headaches is reduced by at least 40%, at least 45%, at least 50%, at least 55%, at least 60%, at least 65%, at least 70%, at least 75%, at least 80%, at least 85%, at least 90%, or at least 95% during the 4-week period immediately following the 28-day initial administration period as compared to the frequency of migraine headaches during the 4 week-period immediately preceding the 28-day initial administration period.

In some embodiments, the subject has at least 10% reduction in migraine headache frequency during the 4-week period immediately following the 28-day initial administration period as compared to the frequency of migraine headaches during the 4 week-period immediately preceding the 28-day initial administration period. In some embodiments, the subject has at least 20%, 30%, 40%, 50%, 60%, or 70% reduction in migraine headache frequency during the 4-week period immediately following the 28-day initial administration period as compared to the frequency of migraine headaches during the 4 week-period immediately preceding the 28-day initial administration period. In some embodiments, the subject has 10-20%, 20-30%, 30-40%, 40-50%, 50-60%, or 60-70% reduction in migraine headache frequency during the 4-week period immediately following the 28-day initial administration period as compared to the frequency of migraine headaches during the 4 week-period immediately preceding the 28-day initial administration period.

In some embodiments, the subject has at least 10% reduction in migraine headache frequency during the 8-week period immediately following the 28-day initial administration period as compared to the frequency of migraine headaches during the 4 week-period immediately preceding the 28-day initial administration period. In some embodiments, the subject has at least 20%, 30%, 40%, 50%, 60%, or 70% reduction in migraine headache frequency during the 8-week period immediately following the 28-day initial administration period as compared to the frequency of migraine headaches during the 4 week-period immediately preceding the 28-day initial administration period. In some embodiments, the subject has 10-20%, 20-30%, 30-40%, 40-50%, 50-60%, or 60-70% reduction in migraine headache frequency during the 8-week period immediately following the 28-day initial administration period as compared to the frequency of migraine headaches during the 4 week-period immediately preceding the 28-day initial administration period.

In some embodiments, the subject has at least 10% reduction in migraine headache frequency during the 20-week period immediately following the 28-day initial administration period as compared to the frequency of migraine headaches during the 4 week-period immediately preceding the 28-day initial administration period. In some embodiments, the subject has at least 20%, 30%, 40%, 50%, 60%, or 70% reduction in migraine headache frequency during the 20-week period immediately following the 28-day initial administration period as compared to the frequency of migraine headaches during the 4 week-period immediately preceding the 28-day initial administration period. In some embodiments, the subject has 10-20%, 20-30%, 30-40%, 40-50%, 50-60%, or 60-70% reduction in migraine headache frequency during the 20-week period immediately following the 28-day initial administration period as compared to the frequency of migraine headaches during the 4 week-period immediately preceding the 28-day initial administration period.

In some embodiments, the reduction in migraine headache frequency lasts at least 4 weeks, 8 weeks, 12 weeks, 16 weeks, 20 weeks, 24 weeks, or more.

In various embodiments, the subject has migraine headache less than three times, twice, or once a month following the 28-day initial administration period. In some embodiments, the subject has no migraine headache during the 4-week immediately following the 28-day initial administration period. In certain embodiments, the subject has migraine headaches less than six times, five times, four times, three times, twice, or once a month during an 8-week period following the 28-day initial administration period. In certain embodiments, the subject has migraine headaches less than twelve times, eleven times, ten times, nine times, eight times, seven times, six times, five times, four times, three times, twice, or once a month during a 12-week period following the 28-day initial administration period. In certain embodiments, the subject has migraine headaches less than eighteen times, seventeen times, sixteen times, fifteen times, fourteen times, thirteen times, twelve times, eleven times, ten times, nine times, eight times, seven times, six times, five times, four times, three times, twice, or once a month during a 24-week period following the 28-day initial administration period. In certain embodiments, the subject has migraine headaches less than three times, twice, or once a month during a 24-week period following the 28-day initial administration period.

In some embodiments, the repeated administrations of the pharmaceutical composition improve pain freedom at 2 hours. In some embodiments, at least 5%, 10%, 15%, 20%, 25%, 35%, 40%, 45%, 50%, 60%, 70%, 80%, 90%, 99% or more subjects experience pain freedom at 2 hours post administration of the pharmaceutical composition using the methods described herein. In some embodiments, the subject achieves a pain relief at 2 hours in at least 20%, 30%, 40%, 50%, 60%, 70%, or 80% of migraines treated with the method provided herein. In some embodiments, improvements of pain freedom at 2 hours sustains for at least 5, 10, 15, 20, 30, 60, or 90 days. In some embodiments, improvements of pain freedom at 2 hours sustains for at least 1, 2, 3, 4, 5, 6, 7, 8, 9, 10, 11, or 12 months. In some embodiments, the benefit with freedom at 2 hours requires 1 dose of administration using the methods described herein. In some embodiments, the frequency of migraine headaches is reduced by at least 75% during the 4-week period immediately following the 28-day initial administration period as compared to the frequency of migraine headaches during the 4 week-period immediately preceding the 28-day initial administration period.

In some embodiments, the repeated administrations of the pharmaceutical composition reduce one or more symptoms selected from pain, nausea, phonophobia, and photophobia. In some embodiments, the repeated administrations of the pharmaceutical composition reduce most bothersome symptom (MBS) at 2 hours post administration. Typical MBS includes, but is not limited to, photophobia, nausea, phonophobia, foggy thinking, vomiting, visual changes, other pain, smell, dizziness, or touch sensitivity. In some embodiments, at least 5%, 10%, 15%, 20%, 25%, 35%, 40%, 45%, 50%, 60%, 70%, 80%, 90%, 99% or more subjects experience most bothersome symptom freedom at 2 hours post administration of the pharmaceutical composition using the methods described herein. In some embodiments, improvement of most bothersome symptom sustains for at least 5, 10, 15, 20, 30, 60, or 90 days. In some embodiments, improvement of most bothersome symptom sustains for at least 1, 2, 3, 4, 5, 6, 7, 8, 9, 10, 11, or 12 months. In some embodiments, the benefit with freedom at 2 hours requires 1 dose of administration using the methods described herein.

In some embodiments, the subject has reduced or no treatment emergent adverse events. In some embodiments, the subject has reduced or no migraine related healthcare utilization. In some embodiments, the subject has reduced or no hospitalizations, emergency room visits and urgent care visits. In some embodiments, the subject has reduced or no headache-related disability as assessed by MIDAS and/or HIT-6 questionnaires.

In some embodiments, the subject has minimal or no change in nasal mucosa as detected by nasal endoscopy, or olfactory function.

In some embodiments, the subject has fewer than 3, fewer than 2, or no migraine headaches as defined by ICHD3b criteria during the 4-week period immediately following the 28-day initial administration period. In some embodiments, the subject has fewer than 6, fewer than 5, fewer than 4, fewer than 3, or fewer than 2 migraine headaches as defined by ICHD3b criteria during the 8-week period immediately following the initial administration period. In some embodiments, the subject has fewer than 12, fewer than 10, fewer than 8, fewer than 6, fewer than 5, fewer than 4, or fewer than 3 migraine headaches as defined by ICHD3b criteria in the 12-week period immediately following the initial administration period. In some embodiments, the subject has fewer than 18, fewer than 16, fewer than 14, fewer than 12, fewer than 10, fewer than 8, fewer than 6, fewer than 5, or fewer than 4 migraine headaches as defined by ICHD3b criteria during the 24-week period immediately following the repeated administrations. In some embodiments, the subject has fewer than 18, fewer than 16, fewer than 14, fewer than 12, fewer than 10, fewer than 8, fewer than 6, fewer than 5, or fewer than 4 migraine headaches as defined by ICHD3b criteria during the 52-week period immediately following the repeated administrations.

### 5.4.4. Pharmaceutical Composition

The present disclosure provides a method of treating a subject with frequent migraine headaches by administering a pharmaceutical composition comprising dihydroergotamine (DHE) or salt thereof.

In various embodiments, the pharmaceutical composition used for the treatment method is a composition suitable for administration via the respiratory system. In some embodiments, the pharmaceutical composition is a liquid composition suitable for intranasal administration, pulmonary administration, or oral inhalation. In some embodiments, the pharmaceutical composition is a dry powder composition suitable for intranasal administration, pulmonary administration, or oral inhalation.

### 5.4.4.1. Liquid Pharmaceutical Composition

The liquid pharmaceutical composition comprises dihydroergotamine (DHE) or salt thereof.

In typical embodiments, the liquid pharmaceutical composition comprises a salt of DHE. In preferred embodiments, the liquid composition comprises DHE mesylate.

Dihydroergotamine mesylate—ergotamine hydrogenated in the 9,10 position as the mesylate salt—is known chemically as ergotaman-3′, 6′, 18-trione, 9,10-dihydro-12′-hydroxy-2′-methyl-5′-(phenylmethyl)-, (5′α)-, monomethane-sulfonate. Its molecular weight is 679.80 and its empirical formula is C₃₃H₃₇N₅O₅.CH₄O₃S. The structure is shown in formula (I) below:

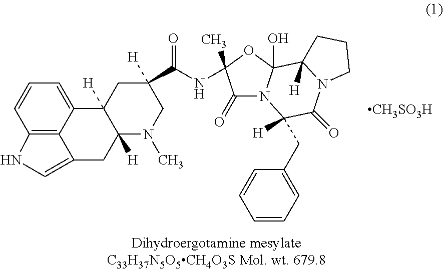

In typical embodiments, the liquid pharmaceutical composition comprises DHE mesylate at a concentration of at least 1 mg/ml, 1.5 mg/ml, 2.0 mg/ml, 2.5 mg/ml, 3.0 mg/ml, 3.5 mg/ml, 4.0 mg/ml, 4.5 mg/ml or 5.0 mg/ml. In some embodiments, the liquid pharmaceutical composition comprises DHE mesylate at a concentration of 2.5-7.5 mg/ml. In certain embodiments, the liquid pharmaceutical composition comprises 3.0-5.0 mg/ml or 3.5-6.5 mg/ml DHE mesylate. In particular embodiments, the liquid pharmaceutical composition comprises 4.0 mg/ml DHE mesylate.

In some embodiments, the composition further comprises caffeine. In particular embodiments, the composition comprises caffeine at a concentration of 1 mg/ml-20 mg/ml, 5 mg/ml-15 mg/ml, or 7.5 mg/ml-12.5 mg/ml. In particular embodiments, the composition comprises 10.0 mg/ml caffeine.

In some embodiments, the composition further comprises dextrose. In certain embodiments, the composition comprises dextrose at a concentration of 5 mg/ml, 10 mg/ml, 15 mg/ml, 20 mg/ml, 25 mg/ml, 30 mg/ml, 35 mg/ml, 40 mg/ml, 45 mg/ml, or 50 mg/ml. In some embodiments, the composition comprises dextrose at a concentration of at least 50 mg/ml.

In various currently preferred embodiments, the liquid pharmaceutical composition comprises 4.0 mg/ml DHE mesylate, 10.0 mg/ml caffeine, and 50 mg/ml dextrose.

### 5.4.4.2. Dry Powder Pharmaceutical Composition

The methods comprise administering to a subject with migraine headache repeated effective doses of a dry pharmaceutical composition comprising dihydroergotamine (DHE) or a salt thereof. In typical embodiments, the dry powder pharmaceutical composition comprises a plurality of particles comprising DHE or a salt thereof, and at least one excipient.

Dry Powder Composition for Intranasal Administration

In some embodiments, the dry pharmaceutical composition is a powder pharmaceutical composition suitable for intranasal administration, the composition comprises an active agent and at least a member selected from the group consisting of a thickening agent, a carrier, a pH adjusting agent, and a sugar alcohol. In some embodiments, at least about 20 percent by powder composition contains 0.1-10 mg, 1-9 mg, 2-7 mg, 3-6 mg, or 4-5 mg of DHE mesylate. In some embodiments, a unit dose of the pharmaceutical composition contains 4-5 mg of DHE mesylate. In some embodiments, a unit dose of the pharmaceutical composition contains 3.9 mg of DHE mesylate. In some embodiments, a unit dose of the pharmaceutical composition contains 5.2 mg of DHE mesylate.

Dry Powder Composition for Pulmonary Administration

In some embodiments, the dry pharmaceutical composition is a powder pharmaceutical composition suitable for pulmonary administration, the composition comprises DHE or a salt thereof and at least one excipient.

In typical embodiments, the powder pharmaceutical composition comprises DHE or a salt thereof, wherein the salt is DHE mesylate, and at least one excipient. In some embodiments, the powder pharmaceutical composition comprises one or more antioxidants. Exemplary formulations of dry powder pharmaceutical composition suitable for pulmonary administration have been described in U.S. Pat. No. 8,119,639, which is incorporated by reference herein in its entirety.

In some embodiments, the composition further comprises additional ingredients, for example preservatives, buffers, tonicity agents, antioxidants and stabilizers, nonionic wetting or clarifying agents, viscosity-increasing agents, absorption enhancing agents, and the like.

Suitable absorption enhancement agents include N-acetylcysteine, polyethylene glycols, caffeine, cyclodextrin, glycerol, alkyl saccharides, lipids, lecithin, dimethylsulfoxide, and the like.

Suitable preservatives for use in a solution include polyquaternium-1, benzalkonium chloride, thimerosal, chlorobutanol, methyl paraben, propyl paraben, phenylethyl alcohol, disodium edetate, sorbic acid, benzethonium chloride, and the like. Typically (but not necessarily) such preservatives are employed at a level of from 0.001% to 1.0% by weight.

Suitable buffers include boric acid, sodium and potassium bicarbonate, sodium and potassium borates, sodium and potassium carbonate, sodium acetate, sodium biphosphate and the like, in amounts sufficient to maintain the pH at between about pH 6 and pH 8, and preferably, between about pH 7 and pH 7.5.

Suitable tonicity agents are dextran 40, dextran 70, dextrose, glycerin, potassium chloride, propylene glycol, sodium chloride, and the like, such that the sodium chloride equivalent of the ophthalmic solution is in the range 0.9 plus or minus 0.2%.

Suitable antioxidants and stabilizers include sodium bisulfite, sodium metabisulfite, sodium thiosulfite, thiourea, caffeine, cromoglycate salts, cyclodextrins and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

In some embodiments, the dry pharmaceutical composition further comprises a stabilizer, wherein the stabilizer is selected from the group consisting of: hydroxypropylmethylcellulose (HPMC), polyvinyl caprolactam-polyvinyl acetate-polyethylene glycol graft co-polymer (Soluplus), vinyl pyrrolinone-vinyl acetate copolymer (Kollidon VA64), polyvinyl pyrrolinone K30 (Kollidon K30), polyvinyl pyrrolidine K90 (Kollidon K90), hydroxypropylcellulose (HPC), hydroxypropyl betacyclodextrin (HPBCD), mannitol, and lactose monohydrate. In some embodiments, the stabilizer is hydroxypropylmethylcellulose (HPMC).

In some embodiments, the dry pharmaceutical composition further comprises an antioxidant, wherein the antioxidant is selected from the group consisting of alpha tocopherol, ascorbic acid, ascorbyl palmitate, bronopol butylated hydroxyanisole (BHA), butylated hydroxytoluene (BHT), citric acid monohydrate, sodium ascorbate, ethylene diainetetraacetic acid, fumaric acid, malic acid, methionine, propionic acid, sodium metabisulfite, sodium sulfite, sodium thiosulfate, thymol, and vitamin E polyethylene glycol succinate.

In some embodiments, the dry pharmaceutical composition further comprises a permeation enhancer, wherein the permeation enhancer is selected from the group consisting of n-tridecyl-B-D-maltoside, n-dodecyl-3-D-maltoside, 1,2-distearoyl-sn-glycero-3-phosphocholine (DSPC), 1,2-dipalmitoyl-sn-glycero-3-phosphocholine (DPPC), 1,2-dioleoyl-sn-glycero-3-phosphocholine (DOPC), propylene glycol, disodium EDTA, PEG400 monostearate, polysorbate 80, and macrogol (15) hydroxystearate. In some embodiments, the permeation enhancer is 1,2-di stearoyl-sn-glycero-3-phosphocholine (DSPC).

In some embodiments, the powder pharmaceutical composition comprises a plurality of particles comprising DHE or a salt thereof and at least one excipient. In some embodiments, the median diameter of the plurality of particles (D50) is 0.5-100, 0.5-75, 0.5-50, 0.5-25, 0.5-10, 0.5-5, or 1-4 μm. In some embodiments, the median diameter of the plurality of particles (D50) is 0.5-75 μm. In some embodiments, the median diameter of the plurality of particles (D50) is 0.5-50 μm. In some embodiments, the median diameter of the plurality of particles (D50) is 0.5-25 μm. In some embodiments, the median diameter of the plurality of particles (D50) is 0.5-10 μm. In some embodiments, the median diameter of the plurality of particles (D50) is 0.5-5 μm. In some embodiments, the median diameter of the plurality of particles (D50) is 1-4 μm.

In some embodiments, the powder pharmaceutical composition is in a crystalline or an amorphous form. In some embodiments, the powder pharmaceutical composition is a partially crystalline and partially amorphous form. In some embodiments, the powder pharmaceutical composition is obtained by spray-drying, supercritical fluid-based process, or prepared using methods that are standard in the field.

### 5.4.5. Route of Administration

Delivery of the pharmaceutical composition can be performed by administration via the respiratory system. Without being bound to any theory, administration via the respiratory system can be delivered by intranasal administration, pulmonary administration, or oral inhalation.

### 5.4.5.1. Intranasal Administration

In some embodiments, each dose of the repeated administration is performed by intranasal administration wherein the composition is a liquid pharmaceutical composition or powder pharmaceutical composition.

### 5.4.5.1.1. Administration of Liquid Composition

In some embodiments, delivery of the liquid composition is performed using an intranasal administration device. In typical embodiments, the intranasal administration device is a manually actuated, propellant-driven, metered-dose intranasal administration device.

In some embodiments, prior to first manual actuation, the liquid pharmaceutical composition and propellant are not in contact within the device. In certain embodiments, the liquid pharmaceutical composition is contained in a vial and the propellant is contained in a canister. The canister may be a pressurized canister. In some embodiments, between successive manual actuations, the liquid pharmaceutical composition in the vial and propellant in the canister are not in contact within the device.

In typical embodiments, each manual actuation brings a metered volume of liquid pharmaceutical composition and a separately metered volume of propellant into contact within a dose chamber of the device, and contact of propellant with liquid pharmaceutical composition within the dose chamber of the device creates a spray of liquid pharmaceutical composition as the formulation is expelled through a nozzle of the device.

In particular embodiments, the nozzle has a plurality of lumens, and the spray is ejected simultaneously through a plurality of nozzle lumens. In some embodiments, the propellant is a hydrofluoroalkane propellant, and in specific embodiments, the propellant is hydrofluoroalkane-134a.

In various embodiments, prior to first actuation, the vial is nonintegral to the device and is configured to be attachable thereto. In some of these embodiments, the vial is configured to be threadably attachable to the device.

In typical embodiments, each of the doses of the liquid composition is administered as two divided subdoses. In a particular embodiment, the divided subdoses are administered into separate nostrils. For instance, the divided subdoses are administered in two sprays, one per nostril. In typical divided subdose embodiments, the dose is administered over no more than 10, 5, 2, or 1 minute. In some embodiments, the divided subdoses are administered within no more than 60, 45, 30, or 15 seconds. In some embodiments, the divided subdoses are administered over no more than 30 seconds. In some embodiments, the divided subdoses are administered within no more than 30 seconds.

### 5.4.5.1.2. Administration of Dry Powder Composition

In some embodiments, delivery of the dry powder composition is performed using an intranasal administration device. In typical embodiments, the intranasal administration device is an intranasally dispenser device.

In some embodiments, the intranasal administration device comprises a nozzle having an upstream end and a downstream end adapted to allow positioning of at least a portion of the nozzle into a nostril of a subject; a reservoir comprising a single dose of a powdered therapeutic formulation, the reservoir having an upstream end and a downstream end, and disposed within the nozzle; a valve having an upstream end and a downstream end, wherein the valve is adapted to occupy a first position and a second position in the device, and wherein the valve is adapted to cause diffusion of the powdered therapeutic formulation when the device is activated; and an air source operably linked to the upstream end of a valve, wherein the device is a single-use device.

In some embodiments, the device comprises an air source that is adapted to be engaged by a user to force air from an air source through a valve assembly into a reservoir and out of a nozzle. In typical embodiments, the device is operated by applying compressive force to a pump. In some embodiments, the pump comprises a manual air pump.

In typical embodiments, each of the doses of the liquid composition is administered as two divided subdoses. In a particular embodiment, the divided subdoses are administered into separate nostrils. For instance, the divided subdoses are administered in two sprays, one per nostril. In typical divided subdose embodiments, the dose is administered over no more than 10, 5, 2, or 1 minute. In some embodiments, the divided subdoses are administered within no more than 60, 45, 30, or 15 seconds. In some embodiments, the divided subdoses are administered over no more than 30 seconds. In some embodiments, the divided subdoses are administered within no more than 30 seconds.

In some embodiments, delivery of the dry powder composition is performed using inhalation therapy. In typical embodiments, the delivery is performed by using a pulmonary administration device. In some embodiments, the device comprises a dry powder inhaler, nebulizer, vaporize, pressurized metered dose inhaler, or breath activated pressurized metered dose inhaler.

### 5.4.5.2. Pulmonary Administration

In some embodiments, each dose of the repeated administration of the dry powder composition is performed by oral inhalation using a pulmonary administration device, wherein each dose is administered via intrapulmonary delivery.

In various embodiments, each dose is administered by a device comprising a dry powder inhaler, nebulizer, vaporizer, pressurized metered dose inhaler, or breath activated pressurized metered dose inhaler. In some embodiments, each dose is administered by a device comprising a breath activated pressurized metered dose inhaler. The breath activated pressurized metered dose inhaler may comprise a plume control feature and/or a vortexing chamber.

In some embodiments, the inhaled dosing is carried out with a breath actuated inhaler such as the Tempo™ Inhaler (Map Pharmaceuticals, Inc., Mountain View, Calif.) as described in U.S. Pat. No. 8,119,639, which is incorporated herein by reference in its entirety.

In some embodiments, the breath actuated pressurized metered dose inhaler contains a suspension of the DHE or salt thereof in a hydrofluoroalkane propellant blend. In some embodiments, the propellant blend consists of 1,1,1,2,3,3,3-heptafluoropropane (HFA 227ea) and 1,1,1,2-tetrafluoroethane (HFA 134a). In a particular embodiment, the propellant blend consists of 70:30 HFA 227ea:HFA 134a.

### 5.4.6. Dose of Administration

The methods comprise repeatedly administering to a subject with migraine headache a plurality of effective doses of a pharmaceutical composition comprising dihydroergotamine (DHE) or a salt thereof, wherein each of the doses is administered by an intranasal delivery device or an intrapulmonary delivery device that provides, following administration of the first dose, (a) a mean peak plasma DHE concentration (Cₘₐₓ) of at least 750 pg/ml, (b) with a mean time to Cₘₐₓ (Tₘₐₓ) of DHE of less than 45 minutes, and (c) a mean plasma AUC_{0-inf} of DHE of at least 2000 pg*hr/ml.

In various embodiments, following administration of the first dose, the mean peak plasma DHE concentration (Cₘₐₓ) achieved following administration of a dose, whether administered as an undivided dose or a plurality of divided subdoses, is at least 750 pg/ml, 800 pg/ml, 900 pg/ml, 1000 pg/ml, 1100 pg/ml, 1200 pg/ml, or 2000 pg/ml. In some embodiments, following administration of the first dose, the mean DHE Cₘₐₓ achieved following administration of a dose is at least 1250, 1300, 1350, 1400, 1450 or 1500 pg/ml. In certain embodiments, following administration of the first dose, the mean DHE Cₘₐₓ achieved following administration of a dose is at least 750 pg/ml, 800 pg/ml, 900 pg/ml, 1000 pg/ml, 1100 pg/ml, or 1200 pg/ml. In certain embodiments, following administration of the first dose, the mean DHE Cₘₐₓ achieved following administration of a dose is at least 1250, 1300, 1350, 1400, 1450 or 1500 pg/ml. In particular embodiments, following administration of the first dose, the mean DHE Cₘₐₓ achieved following administration of a dose is 1000-1500 pg/ml, 1100-1400 pg/ml, or 1200-1300 pg/ml.

In various embodiments, following administration of the first dose, the mean time to Cₘₐₓ (Tₘₐₓ) of DHE following administration is less than 55 minutes. In typical embodiments, the DHE Tₘₐₓ is less than 50 minutes, 45 minutes, 40 minutes, or 35 minutes. In some embodiments, following administration of the first dose, the Tₘₐₓ of DHE following administration is 30-50 minutes, or 35-45 minutes. In particular embodiments, following administration of the first dose, the DHE Tₘₐₓ is no more than 35 minutes, 40 minutes, or 45 minutes. In some embodiments, following administration of the first dose, the DHE Tₘₐₓ is less than 45 minutes. In some embodiments, following administration of the first dose, the DHE Tₘₐₓ is no more than 30 minutes. In some embodiments, following administration of the first dose, the DHE Tₘₐₓ is about 30 minutes.

In various embodiments, the mean plasma AUC_{0-inf} of DHE following administration of the first dose is at least 2500 pg*hr/ml, 3000 pg*hr/ml, 4000 pg*hr/ml, 5000 pg*hr/ml, or 6000 pg*hr/ml. In various embodiments, the mean plasma AUC_{0-inf} of DHE following administration of the first dose is at least 7000 pg*hr/ml, 8000 pg*hr/ml, 9000 pg*hr/ml, or 10,000 pg*hr/ml. In some embodiments, the mean plasma AUC_{0-inf} of DHE following administration of the first dose is at least 5000, 5100, 5200, 5300, 5400, 5500, 5600, 5700, 5800, 5900, or 6000 pg*hr/ml. In some embodiments, the mean plasma AUC_{0-inf} of DHE following administration of the first dose is greater than 6000, 5900, 5800, 5700, 5600, 5500, 5400, 5300, 5200, 5100 or 5000 pg*hr/ml.

In various embodiments, following administration of the first dose, the mean peak plasma concentration (Cₘₐₓ) of 8′-OH-DHE is at least 50 pg/ml. In certain embodiments, the mean Cₘₐₓ of 8′-OH-DHE is at least 55 pg/ml.

In various embodiments, following administration of the first dose, the mean plasma AUC_{0-inf} of 8′-OH-DHE is at least 500 pg*hr/ml. In some embodiments, the mean plasma AUC_{0-inf} of 8′-OH-DHE is at least 600 pg*hr/ml, 700 pg*hr/ml, 800 pg*hr/ml, 900 pg*hr/ml, or even at least 1000 pg*hr/ml. In certain embodiments, the mean plasma AUC_{0-inf} of 8′-OH-DHE is at least 1100 pg*hr/ml, 1200 pg*hr/ml, 1250 pg*hr/ml, 1300 pg*hr/ml, 1400 pg*hr/ml, or 1500 pg*hr/ml. In some embodiments, the mean plasma AUC_{0-inf} of 8′-OH-DHE is at least 1000 pg*hr/ml.

In various embodiments, the dose of a liquid pharmaceutical composition is no more than 2.0 mg DHE or salt thereof. In typical embodiments, the dose is less than 2.0 mg DHE or DHE salt.

In certain embodiments, the dose of a liquid pharmaceutical composition is 1.2-1.8 mg DHE or salt thereof, 1.4-1.6 mg DHE or salt thereof, or 1.4-1.5 mg DHE or salt thereof. In some embodiments, the dose is about 1.2, 1.25, 1.3, 1.35, 1.4, 1.45, 1.5, 1.55, 1.6, 1.65, or 1.7 mg DHE or salt thereof. In some embodiment, the dose is about 1.45 mg DHE or salt thereof.

In various embodiments, the dose of a dry pharmaceutical composition is 0.1-10.0 mg DHE or salt thereof. In typical embodiments, the dose is no more than 10.0 mg DHE or DHE salt.

The pharmaceutical powder composition for intranasal administration is formulated in a unit dose. In certain embodiments, the dose of a dry pharmaceutical composition for intranasal administration is 1.0-6.0 mg DHE or salt thereof, 1.5-4.0 mg DHE or salt thereof, 2.5-4.5 mg DHE or salt thereof, or 4.0-6.0 mg DHE or salt thereof. In some embodiments, the dose is about 1.5, 2.0, 2.5, 3.0, 3.5, 4.0, 4.5, 5.0, 5.5, or 6.0 mg DHE or salt thereof. In some embodiments, the dose is about 3.9 mg DHE or salt thereof. In some embodiments, the dose is about 5.2 mg DHE or salt thereof.

In some embodiments, the powder pharmaceutical composition for intrapulmonary administration is formulated in a unit dose. In some embodiments, a unit dose of the pharmaceutical composition contains 0.1-10, 0.5-5, or 1-2 mg of DHE or a salt thereof. In some embodiments, a unit dose of the pharmaceutical composition contains 0.5-5 mg of DHE or salt thereof. In some embodiments, of the pharmaceutical composition contains 1.0-2.0 mg of a salt of DHE, wherein the salt is DHE mesylate. In some embodiments, a unit dose of the pharmaceutical composition contains 1.0 mg of DHE mesylate.

In some embodiments, the dose is administered as a single undivided dose. In these embodiments, the dose is administered to either the left or right nostril.

In other embodiments, the dose is administered as a plurality of divided subdoses. In some of these embodiments, the dose is administered as 2, 3, or 4 divided subdoses. In particular embodiments, the dose is administered as 2 divided subdoses. In some embodiments, the dose is administered as 2 divided subdoses, with one divided subdose administered to each nostril.

In embodiments in which the dose is administered as a plurality of divided subdoses, the entire effective dose is typically administered over no more than 1 minute—that is, all of the plurality of divided doses are administered within 1 minute of administration of the first divided dose. In certain divided dose embodiments, the dose is administered over no more than 45 seconds. In certain divided dose embodiments, the dose is administered over no more than 30 seconds. In certain divided dose embodiments, the dose is administered over about 30 seconds.

In embodiments in which the dose is administered as a plurality of divided subdoses, the volume of liquid composition administered per divided dose is typically 140-250 μL. In certain embodiments, the volume of liquid composition administered per divided dose is 145 μL-225 μL. In some embodiments, the volume of liquid composition administered per divided dose is 175 μL-225 μL. In particular embodiments, the volume of liquid composition administered per divided dose is about 180 μL or about 200 μL.

### 5.4.7. Additional Embodiments

In further embodiments, the present disclosure provides a method of treating a subject with frequent migraine headaches, with or without aura, comprising administering to the respiratory system of the subject a plurality of doses of a pharmaceutical composition comprising dihydroergotamine (DHE) or salt thereof on a repeat dose schedule, wherein the schedule comprises at least (i) the administration of a first dose of the pharmaceutical composition and (ii) the subsequent administration of a second dose of the pharmaceutical composition within a 28-day initial administration period, and wherein the plurality of doses are sufficient to reduce the frequency of migraine headaches during the 4-week period immediately following the 28-day initial administration period as compared to the frequency of migraine headaches during the 4 week-period immediately preceding the 28-day initial administration period.

Respiratory tract delivery can be effected by intranasal administration or pulmonary administration. Pulmonary administration is used synonymously herein with oral inhalation.

In some embodiments, the schedule comprises the administration of a third dose, a fourth dose of the pharmaceutical composition within the 28-day initial administration period. In some embodiments, the schedule further comprises at least one additional administration following the 28-day initial administration period.

In various embodiments, the schedule comprises administration of multiple doses of the pharmaceutical composition within a certain period of time. In some embodiments, the schedule comprises administration of no more than 12 doses of the pharmaceutical composition within any 28-day period. In some embodiments, the schedule comprises administration of no more than 3 doses of the pharmaceutical composition within any 7-day period. In some embodiments, the schedule comprises administration of no more than 2 doses of the pharmaceutical composition within any 24-hour period.

In some embodiments, the schedule is a chronic intermittent schedule in which each administration is performed while the subject is experiencing a migraine headache.

In some embodiments, the schedule is a fixed schedule in which administrations are performed at prespecified intervals. In some embodiments, the schedule is one administration per week. In some embodiments, the schedule is two administrations per week.

In some embodiments, each dose of the pharmaceutical composition is administered by intranasal administration. In some embodiments, the pharmaceutical composition is a liquid composition. In some embodiments, the pharmaceutical composition is a dry powder composition.

In some embodiments, each of the doses is administered as two divided subdoses. In a particular embodiment, the divided subdoses are administered into separate nostrils. In typical divided subdose embodiments, the dose is administered over no more than 1 minute. In some embodiments, the divided subdoses are administered within no more than 45 seconds, or over no more than 30 seconds. In some embodiments, the divided subdoses are administered within no more than 30 seconds.

The step of delivering can be performed using a delivery device. In some embodiments, the intranasal administration is delivered by an intranasal administration device. In typical embodiments, the intranasal delivery device is a manually actuated, propellant-driven, metered-dose intranasal administration device. In some embodiments, prior to first manual actuation, the liquid pharmaceutical composition and propellant are not in contact within the device. In certain embodiments, the liquid pharmaceutical composition is contained in a vial and the propellant is contained in a canister. The canister may further be a pressurized canister. In some embodiments, between successive manual actuations, the liquid pharmaceutical composition in the vial and propellant in the canister are not in contact within the device.

In some embodiments, each manual actuation brings a metered volume of the pharmaceutical composition and a separately metered volume of propellant into contact within a dose chamber of the device. In some embodiments, contact of propellant with liquid pharmaceutical composition within the dose chamber of the device creates a spray of liquid pharmaceutical composition as the formulation is expelled through a nozzle of the device. In some embodiments, the nozzle has a plurality of lumens, and the spray is ejected simultaneously through a plurality of nozzle lumens. In some embodiments, the propellant is a hydrofluoroalkane propellant. In some embodiments, the propellant is hydrofluoroalkane-134a.

In various embodiments, prior to first actuation, the vial is nonintegral to the device and is configured to be attachable thereto. In some of these embodiments, the vial is configured to be threadably attachable to the device.

In some embodiments, each of the dose is no more than 2.0 mg DHE or salt thereof. In some embodiments, each of the dose is less than 2.0 mg DHE or salt thereof. In some embodiments, each of the dose is about 1.2-1.8 mg DHE or salt thereof. In some embodiments, each of the dose is about 1.4-1.6 mg DHE or salt thereof. In a particular embodiment, the dose is about 1.45 mg DHE or salt thereof.

In a variety of embodiments, the liquid composition is administered as two divided subdoses in two sprays, wherein each of the two divided subdoses is 140-250 pt. In some embodiments, each of the two divided subdoses is 175 μL-225 μL. In some embodiments, the each of the two divided subdoses is about 200 μL.

In typical embodiments, the liquid composition comprises a salt of DHE. In some embodiments, the liquid composition comprises DHE mesylate. In some embodiments, the liquid composition comprises DHE mesylate at a concentration of 2.5-7.5 mg/ml. In some embodiments, the liquid composition comprises DHE mesylate at a concentration of 3.5-6.5 mg/ml. In some embodiments, the liquid composition comprises DHE mesylate at a concentration of 4.0 mg/ml DHE mesylate.

In some embodiments, the liquid composition further comprises caffeine. In some embodiments, the liquid composition comprises caffeine at a concentration of 10 mg/ml. In some embodiments, the liquid composition further comprises dextrose. In some embodiments, the liquid composition comprises dextrose at a concentration of 50 mg/ml. In specific embodiments, the liquid composition comprises 4.0 mg/ml DHE mesylate, 10.0 mg/ml caffeine, and 50 mg/ml dextrose.

In certain embodiments when the pharmaceutical composition is a dry powder composition, the composition is delivered by intranasal administration. In some embodiments, the intranasal administration is delivered by an intranasal dispenser device. In some embodiments, the device comprises an air source that is adapted to be engaged by a user to force air from an air source through a valve assembly into a reservoir and out of a nozzle. In some embodiments, the device is operated by applying compressive force to a pump. In some embodiments, the pump comprises a manual air pump.

In various embodiments, the dry powder pharmaceutical composition comprises DHE or salt thereof and at least one member selected from the group consisting of a thickening agent, a carrier, a pH adjusting agent, and a sugar alcohol.

In some embodiments, the dry powder pharmaceutical composition comprises the thickening agent, wherein the thickening agent is selected from the group consisting of hydroxypropyl methylcellulose (HPMC), hydroxypropyl cellulose, methyl cellulose, carboxymethylcellulose calcium, sodium carboxymethylcellulose, sodium alginate, xanthan gum, acacia, guar gum, locust bean gum, gum tragacanth, starch, carbopols, methylcellulose, and polyvinylpyrrolidone. In a particular embodiment, the thickening agent is HPMC.

In various embodiments, the dry pharmaceutical composition comprises the carrier, wherein the carrier is selected from microcrystalline cellulose, ethyl cellulose, cellulose acetate, cellulose acetate butyrate, cellulose acetate propionate, cellulose acetate phthalate, hydroxypropylmethylcellulose phthalate, starch, chitosan, and βcyclodextrin. In a particular embodiment, the carrier is microcrystalline cellulose.

In various embodiments, the dry pharmaceutical composition comprises the sugar alcohol, wherein the sugar alcohol is selected from the group consisting of mannitol, glycerol, galactitol, fucitol, inositol, volemitol, maltotriitol, maltoetetraitol, polyglycitol, erythritol, threitol, ribitol, arabitol, xylitol, allitol, dulcitol, glucitol, sorbitol, altritol, iditol, maltitol, lactitol, and isomalt. In a particular embodiment, the sugar alcohol is mannitol.

In various embodiments, the dry pharmaceutical composition further comprises a fluidizing agent, wherein the fluidizing agent comprises a calcium phosphate. In some embodiments, the fluidizing agent comprises tribasic calcium phosphate.

In some embodiments, the dry pharmaceutical composition comprises the salt of DHE, wherein the salt is DHE mesylate. In some embodiments, the dry pharmaceutical composition comprises DHE mesylate at a concentration of 0.01-0.2 mg/mg. In some embodiments, the dry pharmaceutical composition comprises DHE mesylate at a concentration of 0.01-0.1 mg/mg. In some embodiments, the dry pharmaceutical composition comprises DHE mesylate at a concentration of 0.016-0.07 mg/mg. In some embodiments, the dry pharmaceutical composition comprises DHE mesylate at a concentration of 0.02-0.07 mg/mg.

In some embodiments, the dry powder pharmaceutical composition comprises DHE mesylate, a first microcrystalline cellulose (MCC-1), a second microcrystalline cellulose (MCC-2), and tribasic calcium phosphate (TCP). In some embodiments, the dry powder pharmaceutical composition comprises DHE mesylate and a first microcrystalline cellulose (MCC-1). In some embodiments, the dry powder pharmaceutical composition comprises DHE mesylate, MCC-1, HPMC, Mannitol, MCC-2 and TCP. In some embodiments, the dry powder pharmaceutical composition comprises DHE mesylate, MCC-1, HPMC and Mannitol. In some embodiments, the dry powder pharmaceutical composition comprises DHE mesylate, MCC-1, HPMC, Mannitol, a pH adjuster, MCC-2 and TCP. In some embodiments, the dry powder pharmaceutical composition comprises DHE mesylate, MCC-1, HPMC, Mannitol and a pH adjuster. In some embodiments, the dry powder pharmaceutical composition comprises DHE mesylate, MCC-1, HPMC and Mannitol.

In some embodiments, the pharmaceutical composition comprises particles having an average diameter from 10-300 μm. In some embodiments, the pharmaceutical composition comprises particles having an average diameter from 15-200 μm. In some embodiments, the pharmaceutical composition comprises particles having an average diameter from 20-100 μm.

In some embodiments, the particles are spray dried, freeze-dried, or melt-extruded. In typical embodiments, the particles are spray dried.

In typical embodiments, the dry pharmaceutical composition is formulated in a unit dose. In some embodiments, the unit dose comprises 3-6 mg of DHE mesylate. In some embodiments, the unit dose comprises 3.9 mg of DHE mesylate. In some embodiments, the unit dose comprises 5.2 mg of DHE mesylate.

In various embodiments, each dose of the dry pharmaceutical composition administered comprises 3-6 mg of DHE or a salt thereof. In some embodiments, the unit dose comprises 3.9 mg of DHE or a salt thereof. In some embodiments, the unit dose comprises 5.2 mg of DHE or a salt thereof.

In some embodiments, each dose of the dry pharmaceutical composition is administered by oral inhalation. In some embodiments, each dose of the dry pharmaceutical composition is administered by a pulmonary administration device. In particular embodiments, each dose is administered via intrapulmonary delivery.

In various embodiments, the dose is administered by a device comprising a dry powder inhaler, nebulizer, vaporizer, pressurized metered dose inhaler, or breath activated pressurized metered dose inhaler. In some embodiments, the dose is administered by a device comprising a breath activated pressurized metered dose inhaler. In some embodiments, the breath activated pressurized metered dose inhaler comprises a plume control feature. In some embodiments, the breath activated pressurized metered dose inhaler comprises a vortexing chamber.

In some embodiments, the breath actuated pressurized metered dose inhaler contains a suspension of the DHE or salt thereof in a hydrofluoroalkane propellant blend. In some embodiments, the propellant blend consists of 1,1,1,2,3,3,3-heptafluoropropane (HFA 227ea) and 1,1,1,2-tetrafluoroethane (HFA 134a). In a particular embodiment, the propellant blend consists of 70:30 HFA 227ea:HFA 134a.

In typical embodiments, each dose comprises 0.1-5.0 mg of DHE or a salt thereof. In some embodiments, each dose comprises 0.1-5.0 mg of DHE mesylate. In some embodiments, each dose comprises 1.0-2.0 mg of DHE mesylate. In some embodiments, each dose comprises 1.0 mg of DHE mesylate.

In some embodiments, the pharmaceutical composition is a dry powder pharmaceutical composition. In certain embodiments, the dry pharmaceutical composition comprises a plurality of particles comprising DHE mesylate and at least one excipient. In some embodiments, the median diameter of the plurality of particles (D50) is 0.5-100 μm. In some embodiments, the median diameter of the plurality of particles (D50) is 0.5-75 μm. In some embodiments, the median diameter of the plurality of particles (D50) is 0.5-50 μm. In some embodiments, the median diameter of the plurality of particles (D50) is 0.5-25 μm. In some embodiments, the median diameter of the plurality of particles (D50) is 0.5-10 μm. In some embodiments, the median diameter of the plurality of particles (D50) is 0.5-5 μm. In some embodiments, the median diameter of the plurality of particles (D50) is 1-4 μm.

In some embodiments, the dry pharmaceutical composition is in a crystalline or an amorphous form. In some embodiments, the dry pharmaceutical composition is a partially crystalline and partially amorphous form. In some embodiments, the dry pharmaceutical composition is in an amorphous form. In some embodiments, the dry pharmaceutical composition is obtained by spray-drying or supercritical fluid-based process.

In some embodiments, following administration of the first dose, the mean peak plasma DHE concentration (Cmax) is at least 750 pg/ml. In some embodiments, following administration of the first dose, the mean peak plasma DHE concentration (Cmax) is at least 1000 pg/ml In some embodiments, following administration of the first dose, the mean peak plasma DHE concentration (Cmax) is at least 1200 pg/ml. In some embodiments, following administration of the first dose, the mean peak plasma DHE concentration (Cmax) is at least 2000 pg/ml.

In some embodiments, following administration of the first dose, the mean time to Cmax (Tmax) of DHE is less than 45 minutes. In some embodiments, following administration of the first dose, the mean time to Cₘₐₓ (Tₘₐₓ) of DHE is no more than 30 minutes. In some embodiments, following administration of the first dose, the mean time to Cₘₐₓ (Tₘₐₓ) of DHE is about 30 minutes.

In some embodiments, following administration of the first dose, the mean plasma AUC_{0-inf} of DHE is at least 2500 pg*hr/ml. In some embodiments, following administration of the first dose, the mean plasma AUC0-inf of DHE is at least 3000 pg*hr/ml. In some embodiments, following administration of the first dose, the mean plasma AUC0-inf of DHE is at least 4000 pg*hr/ml. In some embodiments, following administration of the first dose, the mean plasma AUC0-inf of DHE is at least 5000 pg*hr/ml. In some embodiments, following administration of the first dose, the mean plasma AUC0-inf of DHE is at least 6000 pg*hr/ml. In some embodiments, following administration of the first dose, the mean plasma AUC0-inf of DHE is at least 10000 pg*hr/ml.

In some embodiments, following administration of the first dose, the mean peak plasma concentration (Cmax) of 8′ OH-DHE is at least 50 pg/ml. In some embodiments, following administration of the first dose, the mean Cmax of 8′ OH-DHE is at least 55 pg/ml. In some embodiments, following administration of the first dose, the mean plasma AUC0-inf of 8′ OH-DHE is at least 1000 pg*hr/ml.

In some embodiments, the subject has at least three in the 4-week period immediately preceding the 28-day initial administration period. In some embodiments, the subject has at least four migraine attacks in the 4-week period immediately preceding the 28-day initial administration period.

In some embodiments, the subject has fewer than 3 migraine headaches during the 4-week period immediately following the 28-day initial administration period. In some embodiments, the subject has fewer than 2 migraine headaches during the 4-week period immediately following the 28-day initial administration period. In some embodiments, the subject has no migraine headaches during the 4-week period immediately following the 28-day initial administration period.

In some embodiments, the subject has fewer than 6 migraine headaches during the 8-week period immediately following the initial administration period. In some embodiments, the subject has fewer than 4 migraine headaches during the 8-week period immediately following the initial administration period. In some embodiments, the subject has fewer than 2 migraine headaches during the 8-week period immediately following the initial administration period.

In some embodiments, the subject has fewer than 12 migraine headaches in the 12-week period immediately following the initial administration period. In some embodiments, the subject has fewer than 6 migraine headaches in the 12-week period immediately following the initial administration period. In some embodiments, the subject has fewer than 3 migraine headaches in the 12-week period immediately following the initial administration period.

In some embodiments, the subject has fewer than 18 migraine headaches during the 24-week period immediately following the repeated administrations. In some embodiments, the subject has fewer than 12 migraine headaches during the 24-week period immediately following the repeated administrations. In some embodiments, the subject has fewer than 4 migraine headaches during the 24-week period immediately following the repeated administrations.

In some embodiments, the frequency of migraine headaches is reduced by at least 50% during the 4-week period immediately following the 28-day initial administration period as compared to the frequency of migraine headaches during the 4 week-period immediately preceding the 28-day initial administration period. In some embodiments, the frequency of migraine headaches is reduced by at least 60% during the 4-week period immediately following the 28-day initial administration period as compared to the frequency of migraine headaches during the 4 week-period immediately preceding the 28-day initial administration period. In some embodiments, the frequency of migraine headaches is reduced by at least 75% during the 4-week period immediately following the 28-day initial administration period as compared to the frequency of migraine headaches during the 4 week-period immediately preceding the 28-day initial administration period.

In some embodiments, the administration of the first dose of the pharmaceutical composition reduces one or more symptoms selected from pain, nausea, phonophobia, and photophobia. In some embodiments, wherein reduction of the one or more symptoms occurs at 2 hours post administration.

In some embodiments, the subject has migraine that does not respond to triptan drugs.

In typical embodiments, each of the repeated administrations is performed by a self-administration.

In some embodiments, the repeat dose schedule lasts at least one month. In some embodiments, the repeat dose schedule lasts at least two months. In some embodiments, the repeat dose schedule lasts at least three months. In some embodiments, the repeat dose schedule lasts at least four months. In some embodiments, the repeat dose schedule lasts at least five months. In some embodiments, the repeat dose schedule lasts at least six months.

In some embodiments, the repeat dose schedule lasts 5 to 8 weeks. In some embodiments, the repeat dose schedule lasts 9 to 12 weeks. In some embodiments, the repeat dose schedule lasts 13 to 16 weeks. In some embodiments, the repeat dose schedule lasts 17 to 20 weeks. In some embodiments, the repeat dose schedule lasts 21 to 24 weeks.

In some embodiments, the repeat dose schedule lasts at least 5 weeks. In some embodiments, the repeat dose schedule lasts at least 9 weeks. In some embodiments, the repeat dose schedule lasts at least 13 weeks. In some embodiments, the repeat dose schedule lasts at least 17 weeks. In some embodiments, the repeat dose schedule lasts at least 21 weeks.

In another aspect, the present disclosure provides a method of reducing the frequency of migraine attacks in a subject who has frequent migraine headaches with or without aura, comprising: intranasally administering to the subject a pharmaceutical composition comprising dihydroergotamine (DHE) or salt thereof on a repeat dose schedule, wherein each intranasal administration is delivered by a manually actuated, propellant-driven, metered-dose administration device, and wherein the schedule is a chronic intermittent schedule in which each of the repeated administrations is performed while the subject is experiencing a migraine headache.

In some embodiments, the repeat dose schedule comprises administration of at least a first dose and a second dose of the pharmaceutical composition.

In some embodiments, the first dose and the second dose are administered within a 28-day initial administration period. In some embodiments, the schedule further comprises administration of a third dose of the pharmaceutical composition within the 28-day initial administration period. In some embodiments, the schedule further comprises administration of a fourth dose of the pharmaceutical composition within the 28-day initial administration period. In some embodiments, the schedule further comprises at least one additional administration following the 28-day initial administration period. In some embodiments, the schedule comprises administration of no more than 12 doses of the pharmaceutical composition within any 28-day period. In some embodiments, the schedule comprises administration of no more than 3 doses of the pharmaceutical composition within any 7-day period. In some embodiments, the schedule comprises administration of no more than 2 doses of the pharmaceutical composition within any 24-hour period.

### 5.5. Device

In the methods described herein, the dose is administered by an intranasal delivery device that provides, following intranasal administration, (a) a mean peak plasma DHE concentration (Cₘₐₓ) of at least 750 pg/ml, (b) with a mean time to Cₘₐₓ (Tₘₐₓ) of DHE of less than 45 minutes, and (c) a mean plasma AUC_{0-inf} of DHE of at least 2000 pg*hr/ml.

### 5.5.1. Compound Delivery Device

In various embodiments, the intranasal administration device is a “compound delivery device” as described in U.S. Pat. No. 9,550,036, U.S. Pat. Pub. No. 2018/0256836, or U.S. Pat. Pub. No. 2019/0209463, the disclosures of which are incorporated herein by reference in its entirety.

### 5.5.2. Medical Unit Dose Container Device

In various embodiments, the intranasal administration device is a “medical unit dose container” device as described in WO 2014/179228, U.S. Pat. Pub. No. 2018/0256836, or U.S. Pat. Pub. No. 2019/0209463, the disclosures of which are incorporated herein by reference in its entirety.

### 5.5.3. Device for Intranasal Administration of Liquid Composition: Manually Activated, Propellant-Driven, Metered-Dose Device

In typical embodiments, the intranasal delivery device is a manually actuated, propellant-driven, metered-dose intranasal administration device.

In some embodiments, the liquid pharmaceutical composition and propellant are not in contact within the device prior to first manual actuation, and, optionally, not in contact within the device between successive manual actuations. In such embodiments, the device typically comprises a vial and a canister, wherein the liquid pharmaceutical composition is contained in the vial and the propellant is contained in the canister. Typically, the canister is a pressurized canister of propellant. In typical embodiments, the propellant is a hydrofluoroalkane propellant suitable for pharmaceutical use. In specific embodiments, the propellant is hydrofluoroalkane-134a.

In various embodiments, each manual actuation brings a metered volume of liquid pharmaceutical composition and a separately metered volume of propellant into contact within a dose chamber of the device. Contact of propellant with liquid pharmaceutical composition within the dose chamber of the device propels the dose towards the nozzle of the device, creating a spray as the dose is expelled through the nozzle of the device. In some embodiments, the nozzle has a plurality of lumens, and the spray is ejected simultaneously through a plurality of nozzle lumens.

As discussed in further detail below with respect to kits, in some embodiments the vial is nonintegral to the device and is configured to be attachable thereto. In particular, embodiments, the vial is configured to be threadably attachable to the device.

In some embodiments, the device (e.g., 1123 POD Device) may have a nominal output that is about 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, or 205 μL/actuation pump.

### 5.5.3.1. In-Line Nasal Delivery Device

In certain embodiments, the manually actuated, propellant-driven metered-dose intranasal administration device is an “in-line nasal delivery device” as described in WO 2017/044897, the disclosure of which is incorporated herein by reference in its entirety.

Typically, in these embodiments the device delivers at least a portion of the dose of liquid pharmaceutical composition to the nasal cavity beyond the nasal valve, including delivery to the turbinates and/or the olfactory region. In certain embodiments, the device delivers at least 25%, 30%, 40%, 50%, 60%, or 70% of the dose of liquid pharmaceutical composition beyond the nasal valve. In certain embodiments, the device delivers liquid pharmaceutical composition so that at least 25%, 30%, 40%, 50%, 60%, or 70% of the dose of liquid pharmaceutical composition is brought into contact with the upper third of the nasal cavity (nasal epithelium) of the subject.

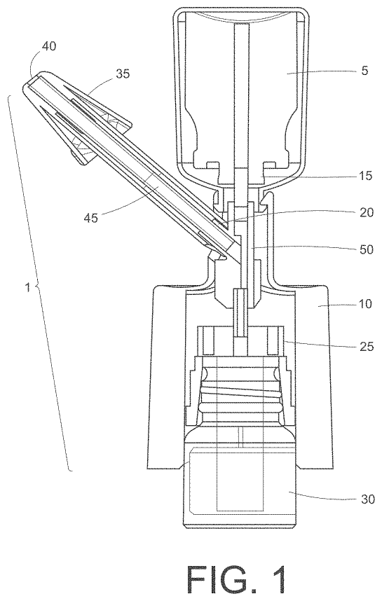

As shown in FIG. 1, the in-line nasal delivery device 1 includes a housing 10, diffuser 20, tip 35, nozzle 40, dose chamber 45, an actuator 50, and a pump 25 to move the liquid pharmaceutical composition into the dose chamber 45. In one series of embodiments, the in-line nasal device 1 is associated and cooperative with a propellant canister 5, a propellant valve 15, and a vial 30 of liquid pharmaceutical composition cooperative with the pump 25 to move the liquid pharmaceutical composition into the dose chamber 45.

In one series of embodiments, the diffuser 20 is a frit 21 (not shown in FIG. 1). The diffuser provides for the conversion of the liquefied propellant in the propellant canister 5 to gas and/or an increase in temperature of the propellant.

In one series of embodiments, the propellant valve 15 is a metered dose propellant valve 16.

In one series of embodiments, the liquid pharmaceutical composition is supplied in the form of a sealed vial 30, e.g., of glass. In one series of embodiments, the vial 30 has a neck 31 (not shown) that is sealed by a removable closure 32 (not shown), for example but not limited to sealed with a plastic cover, crimped metal seal, and rubber stopper (for stability and sterility purposes). When the closure 32 is removed, the device 1 can be engaged with the vial 30. In one series of embodiments, device 1 can be engaged with vial 30 by cooperation with the neck 31 of the vial 30. In a related aspect, further discussed below, sealed vial 30 and device 1 can be co-packaged into a kit to be assembled at time of use.

In certain embodiments, vial 30 is a 3.5-mL amber glass vial.

A pump 25 moves the liquid pharmaceutical composition into the dose chamber 45.

The propellant canister 5 is a canister of a compressed gas or a liquefied propellant. Compressed gases include but are not limited to compressed air and compressed hydrocarbons. In one series of embodiments, the compressed gas is nitrogen or carbon dioxide. Liquefied propellants include but are not limited to chlorofluorocarbons and hydrofluoroalkanes. In some embodiments, propellant canister 5 contains HFA-134a.

The canister 5 will generally be provided with a propellant valve 15 by which the gas flow can be controlled.

The tip 35 includes a nozzle 40. In one series of embodiments, the nozzle 40 has a plurality of nozzle openings 41 (not shown) (synonymously, nozzle lumens). Through the plurality of nozzle openings 41, the liquid pharmaceutical composition and propellant is delivered to the nasal cavity.

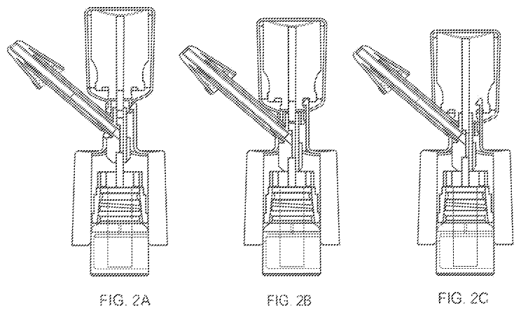

Actuation of the propellant canister 5 is effectively coordinated with actuation of the pump 25 for the vial 30 for the liquid pharmaceutical composition. The arrangement may be such that actuation of the vial 30 for the liquid pharmaceutical composition causes actuation of the propellant canister 5. FIGS. 2A, 2B and 2C show the device 1 at rest (FIG. 2A) and in actuation (FIG. 2B and FIG. 2C).

As an example, the staging of the device 1 actuation is as follows. The housing 10 is compressed to prime the propellant canister 5. When the housing 10 is compressed, an actuator 50 remains stationary in the housing 10 while the propellant canister 5 and the vial 30 move towards the actuator 50. At this time, the propellant valve 15 associated with the propellant canister 5 is not actuated by compression. The actuator 50 acts upon the pump 25 compressing the pump 25 and the liquid pharmaceutical composition from the vial 30 is moved into the dose chamber 45. After a majority of the liquid pharmaceutical composition has moved into the dose chamber 45, the actuator 50 acts upon the propellant valve 15 and the propellant valve 15 begins to compress. The continued depression of the actuator 50 releases the propellant from the propellant canister 5. The propellant pushes the liquid pharmaceutical composition as it exits the device 1 through the nozzle openings (lumens) 41 (not shown) of the nozzle 40 located in the tip 35. The actuator 50 provides for first actuation of the pump 25, then once the pump 25 bottoms out, the continued depression of the actuator 50 provides for release of the propellant from the canister 5.

In an alternative implementation of the device 1 (not shown), the device 1 does not include a diffuser 20. In such embodiments, the device typically incorporates another type of dose retaining valve.

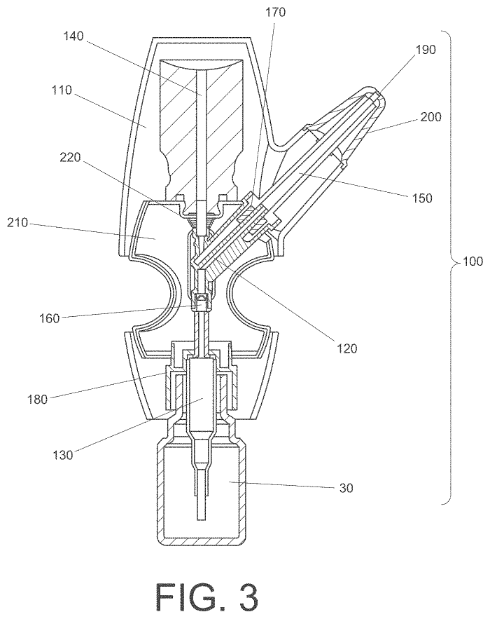

FIG. 3 shows yet another implementation of the device 100. The device 100 can deliver a single or multiple dose from a vial 30 or other container. The device 100 allows for multiple doses to be delivered from the vial 30, or a single dose. For example, the vial 30 may contain a volume of liquid pharmaceutical composition for multiple doses, while the user may decide to only deliver a single dose from the vial 30. The liquid pharmaceutical composition may be a drug, active pharmaceutical ingredient, or a pharmaceutical formulation.

Initially, the vial 30 may be separate from the rest of the assembled device 100. At the time of use, the device 100 and vial 30 are taken out of their respective packaging. Prior to use, the vial 30 will generally be sealed. In the embodiment where the vial 30 is covered by a plastic cover, metal seal and stopper, the plastic cover and metal seal are pulled away from the top of the vial 30, and the rubber stopper is removed from the vial 30. The vial 30 may be screwed into a pump fitment 180 located at the base of the device 100. For example, but not limitation, the vial 30 may have female threads which can be screwed into male threads on a pump fitment 180, or vice versa. The vial 30 may contain, for example but not limited to, inclusive of end points, 2-3 ml, in another embodiment 2-2.5 ml of liquid pharmaceutical composition.

As shown in FIG. 3, the device 100 includes a housing 110. The housing 110 contains components of the device 100 including the Y-junction 120. The Y-junction 120 has three branches radiating from a common base. The Y-junction and its three branches may be a molded component. The Y-junction 120 establishes both fluid and gas paths within the device 100, and connects the metered dose pump 130, the dose chamber 150, and the propellant canister 140 when the propellant canister 140 is assembled with the device.

As shown in FIG. 3, for use of the device 100, the user will generally orient the device 100 with the propellant canister 140 assembled and located at the top and the vial 30 assembled and located at the bottom. Housed within the device's 100 housing 110, the optional check-valve 160 (attached to the metered dose pump 130 stem) press fits into a receiving hub of a first branch of the Y-junction 120. An internal bore provides fluid communication from the metered dose pump 130, through the optional check-valve 160 and to a third branch of the Y-junction 120, which connects to the dose chamber 150. In one series of embodiments, the check valve 160 is an elastomeric component that installs within a plastic housing between the metered dose pump 130 and the Y-junction 120. The optional check valve 160: (a) reduces or eliminates dose leakage which could occur through the metered dose pump 130 if the pump stem was depressed and the propellant canister 140 was actuated; (b) allows for improved consistency in dose delivery by the device 100; and/or provides that liquid pharmaceutical composition is not pushed back down the internal dose loading channel 230 of the Y-junction 120 and into the metered dose pump 130.

When oriented as to be used in operation, housed within the device's 100 housing 110, towards the top of the device 100, the propellant canister 140 press fits into a second branch of the Y-junction 120, establishing the gas path through internal bores, through the diffuser 170 and to the dose chamber 150.

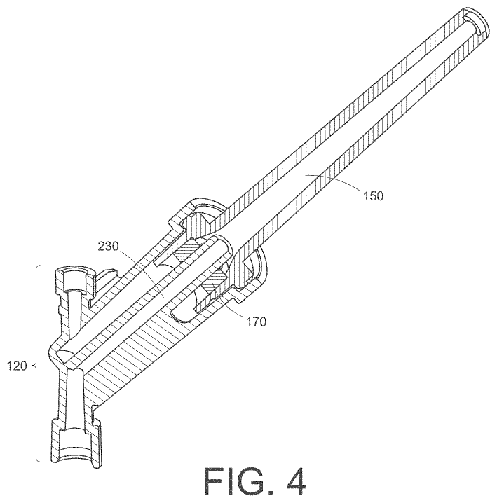

In this implementation of the device 100, the diffuser 170 is annular. As shown in FIG. 4, the annular diffuser 170 sits inside a bore on the back end of the dose chamber 150. The external diameter of the annular diffuser 170 is in a compression fit with the dose chamber 150. In other embodiments, not shown, the annular diffuser is fixed to the dose chamber using means other to or in addition to compression fit.

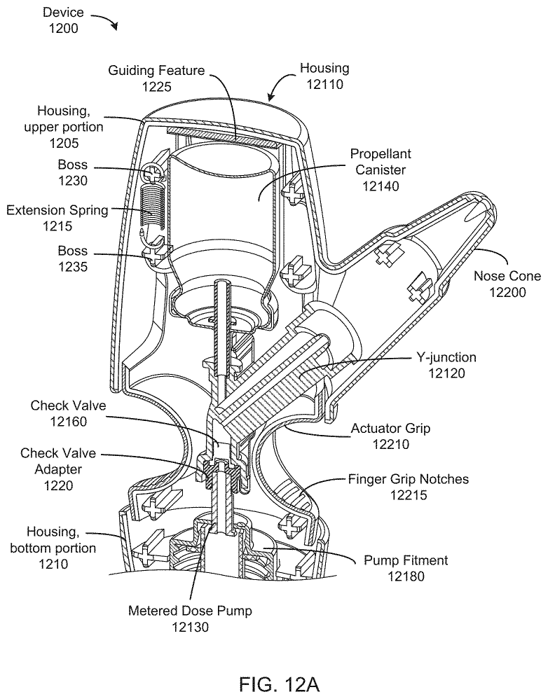

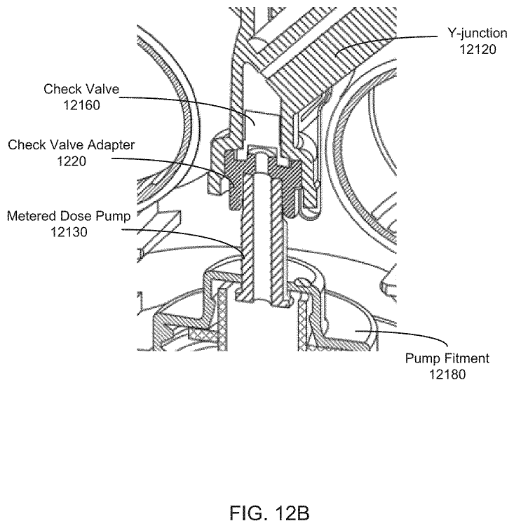

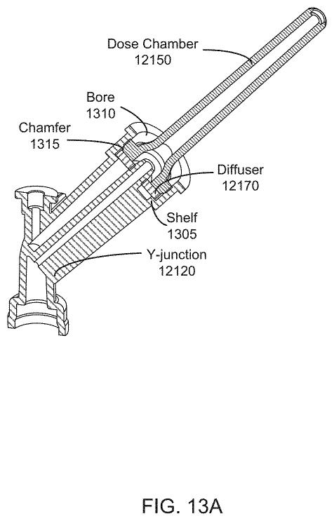

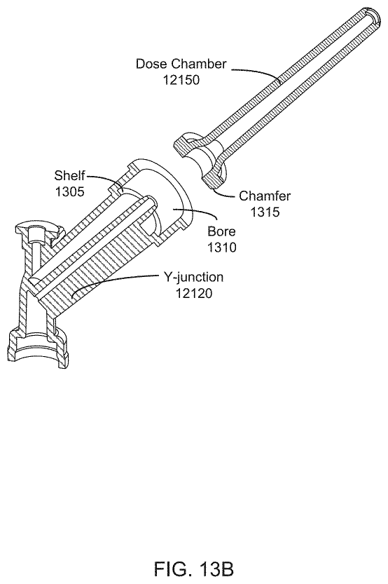

An internal dose loading channel 230 which is molded as a portion of the Y-junction 120 fits into the inner bore of the annual diffuser 170 when the dose chamber 150 is installed onto the Y-junction 120. The inner diameter of the annular diffuser 170 is in compression with the internal dose loading channel 230 portion of the Y-junction 120. The annular diffuser 170 is seated between the outer wall of the internal dose loading channel 230 and the inner wall of the dose chamber 150, sealing against both of those surfaces to form the bottom of the dose chamber 150. Additional embodiments of the diffuser 170, dose chamber 150, and Y-junction 120 are discussed with regards to FIGS. 12-13.

In one series of embodiments, the diffuser 170 is a frit 171 (not shown). In other embodiments, the diffuser 170 is a component that is homogenously or heterogeneously porous. In some embodiments, the diffuser 170 may be a disk-shaped member. The diffuser 170: (a) provides for the conversion of the liquefied propellant in the propellant canister 140 to gas; (b) provides an increase in temperature of the propellant; (c) acts to prevent the propellant from flowing back into the device 100; (d) acts to prevent the liquid pharmaceutical composition from flowing back into the device 100; and/or (e) acts to allows gas flow into the dose chamber 150 while preventing the liquid pharmaceutical composition from leaking out. The diffuser may be made of a porous polymer material.

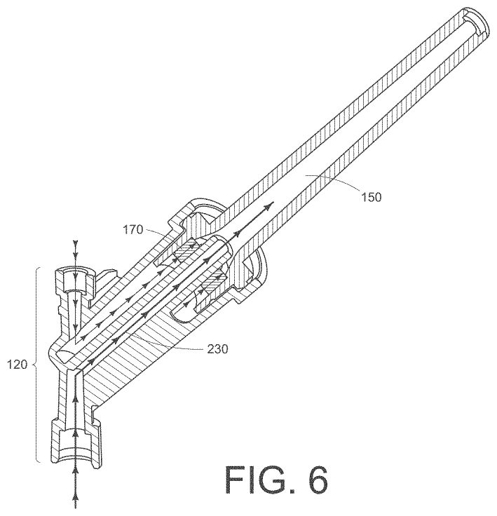

The relationship in operation of the device 100 between the liquid pharmaceutical composition, the diffuser 170, the inner dose loading tube 230, the dose chamber 150 and the Y-junction 120 are shown at least in FIG. 6. In operation, the liquid pharmaceutical composition being loaded into the dose chamber 150 takes the less restrictive route, flowing out of the vial 30 and filling the dose chamber 150 rather than loading backwards through the diffuser 170 and into the delivery path of the propellant of the Y-junction 120. In operation of the device 100, the staging of operation and the amount of time required for operation of the device allows the diffuser 170 to restrict liquid pharmaceutical composition from flowing back into the Y-junction 120 for the period of time needed, as the propellant canister 140 is activated after liquid pharmaceutical composition loading. During proper device 100 use, the entire actuation of the device 100, including metered dose pump 130 and propellant canister 140, is approximately a second or less than a second. The loaded dose in the dose chamber 150 does not have enough time to flow backwards into the Y-junction 120. Immediately after the dose chamber 150 is full, the propellant expels the liquid pharmaceutical composition from the device 100.

On the third leg of the Y-junction 120 at a 45-degree angle, the dose chamber 150 press fits into the Y-junction 120, completing the flow paths for both gas and fluid through the device. In one series of embodiments, the angle is 30 degrees, 35 degrees, 40 degrees, 45 degrees, 50 degrees, 55 degrees, 60 degrees, inclusive of endpoints and intervening degrees.

The Y-junction 120 may contain engagement ribs (not shown) to help secure and position the assembly within the housing 110 of the device 100.

The device 100 includes a pump fitment 180. The pump fitment 180 secures the metered dose pump 130 to the vial 30 and holds both components in place during device 100 use. One series of embodiments of the pump fitment 180 is that it consists of engagement ribs that retain it within the housing 110, provide vertical displacement, and prevent rotation during installation of the vial 30.

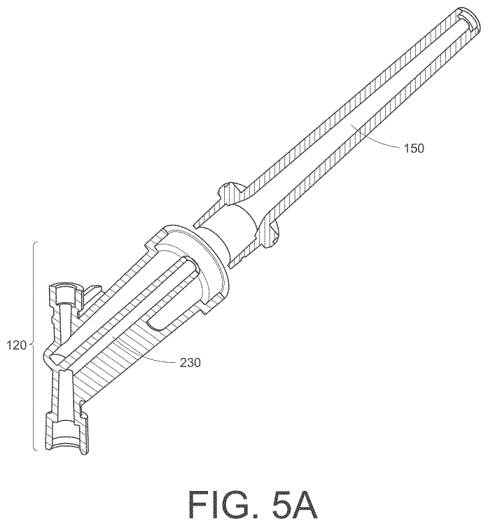

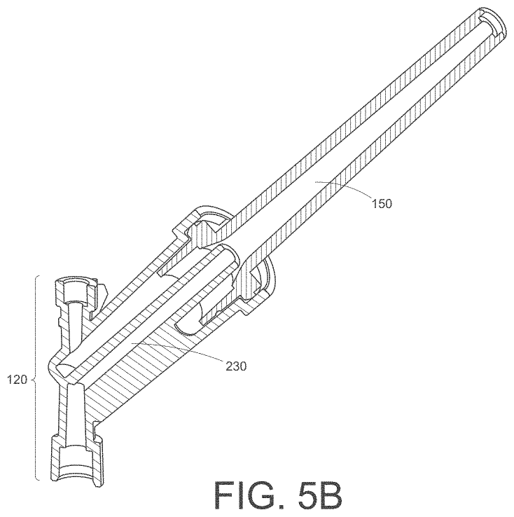

The device 100 includes a dose chamber 150. The dose chamber 150 receives and stores the liquid pharmaceutical composition that has been pushed out of the inner tube of the Y-junction 120. When the propellant canister 140 is actuated, the Y-junction 120 and dose chamber 150 are pressurized and the propellant gas expels the liquid pharmaceutical composition out of the dose chamber 150. As shown in FIGS. 5A and 5B, the dose chamber 150 is press fit into the Y-junction 120. The nozzle 190 is installed into the end of the dose chamber 150 opposite where it is press fit into the Y-junction 120.

The nozzle 190 is installed into the distal end (end opposite where the dose chamber 150 is press fit into the Y-junction 120) of the dose chamber 150, forming a liquid and gas-tight seal around the outer diameter. During actuation of the device 100, propellant evacuates liquid pharmaceutical composition from the dose chamber 150, pushing it out the nozzle 190.

The nozzle 190 forms the narrow plume angle (for example, an angle of 1 to 40 degrees, including endpoints and angles intermittent there between; in one series of embodiments the angle is 5 degrees, 10 degrees, 15 degrees, 20 degrees, 25 degrees, 30 degrees, 35 degrees) multi-stream deposition. The nozzle 190 and resultant angle of the plume produced promotes delivery of the liquid pharmaceutical composition to the olfactory region of the user's nasal cavity.

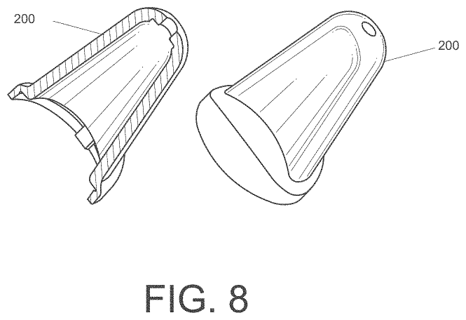

In this implementation, as shown in FIG. 8, the device 100 may include an optional nose cone 200. The external geometries of the nose cone 200 assist in providing proper alignment of the device 100 during insertion into the nose. The diametrically opposed flat sides aid with placement against the septum of either naris, with the depth stop providing correct depth of insertion. The nose cone 200 adds redundancy to nozzle 190 retention through mechanical interference incorporated into the design. As shown in FIG. 3 and FIG. 8, there is an opening in the nose cone 200 which aligns with the nozzle 190. The nose cone 200 is not part of the pressurized flow path.

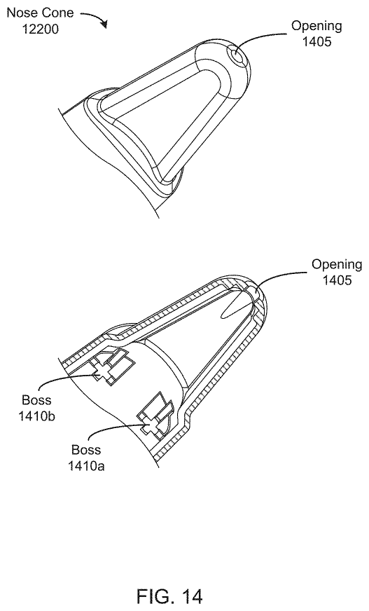

The housing 110 represents the body of the device 100. The housing 110 includes two different “clamshells” concealing the components of the device 100 and retaining all components to ensure functionality. The housing 110 houses the metered dose pump 130 and pump fitment 180, the actuator grip 210, the Y-junction 120, the propellant canister 140, and the dose chamber 150. The nose cone 200 engages onto the outer geometry of the housing 110, or may be optionally integrated into the design of the clamshells. An additional embodiment of the nose cone 200 is discussed with regards to FIG. 14. The housing 110 is designed to assemble easily through the use of, for example but not limited to, mattel pins, snaps, post or screws, or a combination thereof, molded into the geometry.

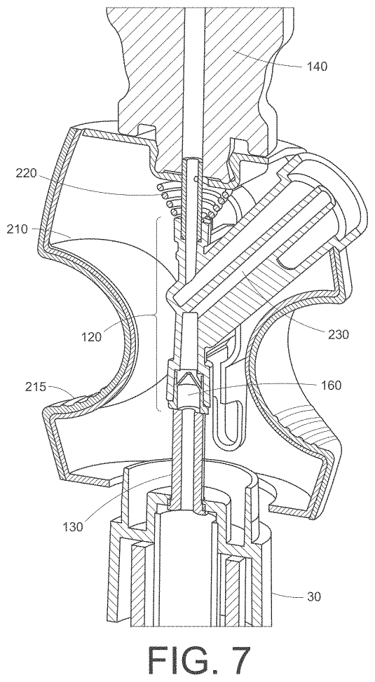

The actuator grip 210 provides for actuation displacement by the user. The actuator grip 210 is composed of two parts, actuator grip A and actuator grip B and surround the Y-junction 120 and reside within the housing 110. FIG. 7 shows two finger grip notches 215 are designed into the actuator grip 210 to allow the user to engage the device 100 with the fingers, for example but not limited to, the index and middle finger. These finger grip notches 215 allow the user to apply downward movement leading to device 100 actuation.

The metered dose pump 130 draws liquid pharmaceutical composition up from the vial 30 to the Y-junction 120. The metered dose pump 130 may utilize a custom pump fitment 180 to promote functionality within the device 100, and allow attachment of the vial 30 via threads. The metered dose pump 130 may deliver, for example but not limited to, volumes of 130 μl, 140 μl, 150 μl, 160 μl, 170 μl, 180 μl, 190 μl, 200 μl, or 230 μl during actuation. Commercially available metered dose pumps 130 can be used.

For the device 100 to consistently deliver liquid pharmaceutical composition, the metered dose pump 130 must first deliver liquid pharmaceutical composition, followed by propellant canister 140 actuation to expel the liquid pharmaceutical composition. As shown in FIG. 7, one manner in which to accomplish this is via a conical spring 220 between the propellant canister 140 and Y-junction 120 to create the necessary propellant canister 140 actuation force resulting in the correct order of actuation between the metered dose pump 130 and propellant canister 140. In one implementation, a conical spring 220 is used, although this force is not limited to being produced by a conical spring 220 as other mechanisms can be used. In one series of embodiments, the conical spring 220 has a near zero preload, with a k value of about 25.5 lbf in and a maximum load of 3.2 lbf. Selection of the spring or mechanism will include the considerations of: (a) providing for proper device 100 staging; (b) physical space in the device 100; and/or (c) and user feedback regarding how stiff of a conical spring 220 still allows a variety of users to activate the device 100.

The conical spring 220 is installed inline between the propellant canister 140 and Y-junction 120. The actuator grip 210 physically holds the propellant canister 140. The user activates the device 100 by, for example, applying an in-line force acting down from the actuator grips 210, and up from the vial 30. This force simultaneously acts to activate both the metered dose pump 130 and the propellant canister 140. The conical spring 220 acts in parallel to the internal propellant canister metering valve spring, increasing the necessary force required to activate the propellant canister 140. By choosing the conical spring 220 such that the necessary force required to actuate the propellant canister 140 is in excess of the maximum necessary force required to completely actuate the metered dose pump 130, the device 100 provides that dose is loaded into the dose chamber 150 before propellant gas begins to expel liquid pharmaceutical composition from the device 100.

In another embodiment, an extension spring is used in lieu of a conical spring. The extension spring is discussed with regards to FIG. 12A.

During device 100 actuation, the metered dose pump 130 draws liquid pharmaceutical composition up from the vial 30 at the bottom of the device 100 via the Y-junction 120, through the internal dose loading channel 230 and into the dose chamber 150. The internal dose loading channel 230 provides a clear route for the liquid pharmaceutical composition to be loaded ahead of the diffuser 170, without needed to physically pass through the porous material of the diffuser 170. As shown in FIG. 6, small arrow heads represent the flow of the propellant while large arrow heads represent the flow of the liquid pharmaceutical composition. Priming shots may be required to completely fill the metered dose pump 130 and internal dose loading channel 230 of the Y-junction 120 prior to user dosing. An optional dose cap (not shown) may cover the nose cone 200 of the device 100 and captures the priming shots while also providing a means of visual indication to the user that the device is primed.

In the second stage of device 100 actuation, once the dose chamber 150 has been filled, the propellant canister 140 releases propellant which enters through the top of the Y-junction 120, following the path shown by smaller arrow heads in FIG. 6. The propellant flows physically through the porous material of the diffuser 170, which promotes the vaporization of the propellant. The diffuser 170 and the path along which the propellant travels (shown by the arrow heads in FIG. 6) convert liquid propellant into gas propellant, resulting in expansion and propulsion of the propellant. The propellant first contacts the liquid pharmaceutical composition at the proximal (distal being closer to the nozzle 190, proximal being farther away from the nozzle 190) face of the diffuser 170 as seated in the device 100. As the propellant continues to expand, it pushes the liquid pharmaceutical composition forward (toward the nozzle 190) in the dose chamber 150, exiting though the nozzle 190 at the end of the dose chamber 150.

The propellant canister 140 provides the propulsive energy for the device 100. The stem of the propellant valve seats into the top receiver of the Y-junction 120. During use, the user presses down on the actuator grips 210 which pulls the propellant canister 140 body down, actuating the propellant valve. This releases a metered volume of liquid propellant. As the propellant vaporizes and expands, the liquid pharmaceutical composition is forced toward the distal end of dose chamber 150 and out through the nozzle 190.

As a non-limiting example of propellant, the propellant canister 140 uses HFA 134A as the propellant for the system. Other propellants are envisioned. There are commercially available propellant canisters 140.

In certain embodiments, the device, propellant canister, and vial containing liquid pharmaceutical composition are provided separately, optionally co-packaged into a kit, and thereafter assembled for use. In certain embodiments, propellant canister 140 is provided assembled within device 100 and the vial containing liquid pharmaceutical composition is provided separately, optionally with the device (with integrated canister) and vial co-packaged into a kit. In some embodiments, the device, propellant canister, and vial containing liquid pharmaceutical composition are provided to the user fully assembled.

### 5.5.3.2. Alternate in-Line Nasal Delivery Device

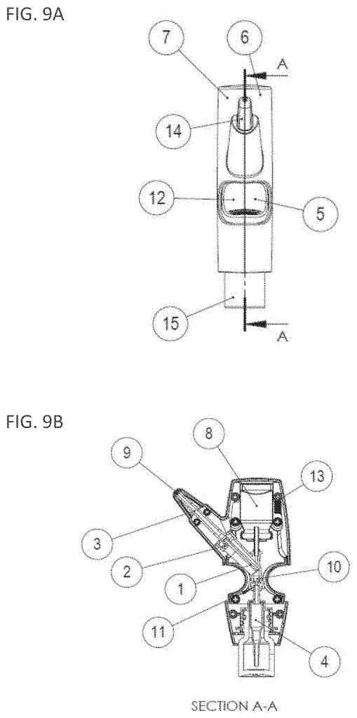

In certain embodiments, the device comprises the following parts; part numbering is as depicted in FIGS. 9A and 9B.

TABLE 1
Clinical Trial Device

| COMPONENT | PART ID | PART NAME | MATERIAL |
|---|---|---|---|
| Device | 1 | Y-Junction | PP |
| | 2 | Diffuser | PE |
| | 3 | Dose Chamber | PP |
| | 4 | Metering Pump | POM; PE Medium Density; Chlorobutyl |
| | | | Rubber PP; White Masterbatch Colorant |
| | | | Stainless Steel; PE (HDPE + LDPE) |
| | 5 | Finger Grip (right) | ABS |
| | 6 | Clamshell (right) | ABS |
| | 7 | Clamshell (left) | ABS |
| | 8 | Propellant Canister | Propellant: HFA |
| | | | Canister: Anodized Aluminum |
| | | | HFA Metering Valve: Anodized |
| | | | Aluminum; Polyester; Stainless Steel; |
| | | | EF327 Seat and Gasket |
| | 9 | Nozzle | LCP |
| | 10 | Check Valve | Silicone |
| | 11 | Check Valve Adapter | PP |
| | 12 | Finger Grip (left) | ABS |
| | 13 | Extension Spring | Stainless Steel |
| | 14 | Nose Cone | ABS |
| Drug | 15 | Drug Vial | 3.5 ml amber glass vial container |

Abbreviations
ABS = acrylonitrile butadiene styrene;
CMO = contract manufacturing organization;
HDPE = high density polyethylene;
HFA = hydrofluoroalkane-134a;
LCP = liquid crystal polymer;
LDPE = low density polyethylene;
PE = polyethylene;
POM = polyacetal copolymer;
PP = polypropylene

The vial contains liquid pharmaceutical composition in an amount sufficient for at least one total dose of DHE, or salt thereof, to be delivered by the device, in a single undivided or a plurality of divided doses. In particular embodiments, the vial contains liquid pharmaceutical composition in an amount sufficient for at most one total dose of DHE, or salt thereof, to be delivered by the device, in a single undivided or a plurality of divided doses.

In various embodiments, the propellant canister contains pressurized propellant in an amount sufficient for optional priming of the device followed by delivery of at least one total dose of DHE, or salt thereof, to be delivered by the device, in a single undivided or a plurality of divided doses. In particular embodiments, the propellant canister contains pressurized propellant in an amount sufficient for optional priming of the device followed by delivery of at most one total dose of DHE, or salt thereof, to be delivered by the device, in a single undivided or a plurality of divided doses.

In some embodiments, with each actuation, a minority of the pressurized liquid hydrofluoroalkane is converted to gaseous hydrofluoroalkane. In certain embodiments, the quantity of pressurized liquid hydrofluoroalkane is sufficient to permit a predetermined number of device actuations. In some of these embodiments, the quantity is sufficient to permit 2, 3, 4, 5, 6, 7 or 8 actuations. In some embodiments, the quantity is sufficient to permit 10, 11, 12, 13, 14, 15, or even 20 actuations. In certain embodiments, a majority of the pressurized liquid hydrofluoroalkane is converted to gaseous hydrofluoroalkanes after 2, 3, 4, 5, 6, 7, or 8 actuations. In certain embodiments, a majority of the pressurized liquid hydrofluoroalkane is converted to gaseous hydrofluoroalkanes after 10, 11, 12, 13, 14, 15, or 20 actuations.

FIG. 12A shows a cross section of an alternate implementation of the in-line nasal delivery device 1200. The in-line nasal delivery device 1200 may be an embodiment of the in-line nasal delivery device 100. For example, the device 1200 may use the same or similar components as the device 100, as described with regards to FIGS. 3-9. Additionally, components of device 1200 and device 100 may be used interchangeably or in some combination thereof. In the embodiment of FIG. 12A, the device 1200 includes a housing 12110, a Y-junction 12120, a metered dose pump 12130, a propellant canister 12140, a dose chamber 12150 (shown in FIG. 13A), a check valve 12160, a diffuser 12170 (shown in FIG. 13A), a pump fitment 12180, a nozzle (not shown), a nose cone 12200, and an actuator grip 12210. The housing 12110 includes an upper portion 1205 and a bottom portion 1210. The device 1200 additionally includes an extension spring 1215 and a check valve adapter 1220.

Similar to the actuator grip 210 described with regards to FIG. 3, the actuator grip 12210 provides for actuation displacement by the user. The actuator grip 12210 surrounds the Y-junction 12120 and resides within the housing 12110. FIG. 12A shows two finger grip notches 12215 that are designed into the actuator grip 12210 to allow the user to engage the device 1200 with the fingers, for example but not limited to, the index and middle finger. The finger grip notches 12215 allow the user to engage or grip the device in order to cause device 1200 actuation.

More specifically, the actuator grip 12210 includes a guiding feature 1225 that extends along a length of the housing 12110 behind (as illustrated in FIG. 12A) the propellant canister 12140 and captures an end of the propellant canister 12140. In the illustrated example, the end is the bottom of the propellant canister 12140, which is opposite from the end containing the valve for propellant dispersal. The guiding feature 1225 may capture the end of the propellant canister 12140 by folding above or adhering to the end. The propellant canister 12140 is nested within the guiding feature 1225 such that the guiding feature 1225 securely supports the propellant canister 12140. By enveloping a portion of the propellant canister 12140, the guiding feature 1225 is securely coupled to a larger, more rigid surface area of the propellant canister 12140 than when coupled to a narrow surface, such as the propellant valve 15 in the embodiment of device 1. In this configuration, as the user applies downward movement via the finger grip notches 12215 to actuate the device 1200, the guiding feature 1225 transmits the downward force to the propellant canister 12140, thereby actuating the propellant canister 12140. The guiding feature 1225 actuates the propellant canister 12140 in a stable manner and is less likely to lose its physical coupling to the propellant canister 12140.

In one embodiment, the propellant canister 12140 is entirely enclosed within the housing 12110. In one specific embodiment, the propellant canister 12140 is enclosed by the upper portion of the housing 1205, which may be formed during manufacturing from at least two separate parts. The Y-junction 12120 is fixed in place with the bottom housing portion 1210, with the guiding feature 1225 extending upward to establish the position of the propellant canister 12140 with respect to the Y-junction 12120. This structure ensures that the propellant canister 12140 moves relative to the Y-junction 12120 during actuation, to which it is fluidly coupled.

In a similar manner to the conical spring 220 described with regards to FIG. 7, the extension spring 1215 creates an actuation force that ensures a desired order of actuation between the metered dose pump 12130 and the propellant canister 12140. Specifically, during device actuation, the metered dose pump 12130 first delivers liquid pharmaceutical composition to the dose chamber 12150, followed by propellant canister 12140 actuation to expel the liquid pharmaceutical composition. The force of the extension spring 1215 is established to both provide proper order of actuation and enable ease of actuation by users.

The extension spring 1215 is coupled to the housing upper portion 1205 and the actuator grip 12210. As illustrated in FIG. 12A, a first end of the extension spring 1215 couples to a boss 1230 on the housing upper portion 1205, and a second end of the extension spring 1215 couples to a boss 1235 on the actuator grip 12210. In the embodiment of FIG. 12A, the housing upper portion 1205 and the actuator grip 12210 translate relative to one another during actuation of the device 1200. The extension spring 1215 is coupled to each component such that the extension spring 1215 creates a resisting force when the housing upper portion 1205 and the actuator grip 12210 translate away from each other. As previously described, the user activates the device 1200 by, for example, applying an in-line force acting down from the actuator grips 12210, and up from the vial containing the pharmaceutical composition. This applied force actuates both the metered dose pump 12130 of the vial and the propellant canister 12140. As the applied force on the extension spring 1215 increases, a threshold (higher) force to actuate the propellant canister 12140 is achieved after a threshold (lower) force to actuate the metered dose pump 12130 is achieved, such that the applied force first exceeds the threshold force of the metered dose pump 12130. In this configuration, actuation of the device 1200 first activates the metered dose pump 12130 and then activates the propellant canister 12140 such that dose is loaded into the dose chamber 12150 before propellant begins to expel liquid pharmaceutical composition from the device 1200.

In some embodiments, the extension spring 1215 may be used in lieu of or in addition to the conical spring 220. The configuration of the extension spring may streamline the assembly process of the device relative to the configuration of the conical spring, as the conical spring may create a resisting force between the propellant canister 140 and Y-junction 120 such that the components are pushed apart during assembly, whereas the extension spring may pull the components towards each other. In addition, the configuration of the extension spring may prolong the shelf life and overall lifetime of the device relative to the configuration of the conical spring. This may be in part due to the press fit between the stem of the propellant canister 140 and Y-junction 120 of the device 100, which may naturally relax over time and which may be propagated by the resisting force of the conical spring between the propellant canister 140 and Y-junction 120, potentially furthering the decrease in durability of the press fit over time.

The check valve adapter 1220 is an adapter that couples the check valve 12160 and the Y-junction 12120. The check valve 12160 may be an embodiment of check valve 160. In the embodiment of FIGS. 12A-12B, the check valve adapter 1220 is a cylindrical component having a first end that inserts into a channel of the Y-junction 12120 and mates with the check valve 12160 positioned within the channel of the Y-junction 12120 and a second end that mates with the metered dose pump 130. As illustrated in the zoomed-in view in FIG. 12B, an end of the check valve 12160 comprises a flange that is captured at an end of the channel of the Y-junction 12120 and mates with a respective interface of the check valve adapter 1220. The check valve 12160 and/or check valve adapter 1220 may be secured at each end with an adhesive, ultrasonic welding, an interference fit (e.g., press fit, friction fit, or similar), or some combination thereof. The check valve adapter 1220 may augment the function of the check valve 12160 by improving the seal between the check valve 12160 and the Y-junction 12120. As discussed with regards to FIG. 3, a check valve may: (a) reduce or eliminate dose leakage which could occur through the metered dose pump if the pump stem was depressed and the propellant canister was actuated; (b) allow for improved consistency in dose delivery by the device; and/or (c) provide that liquid pharmaceutical composition is not pushed back down an internal dose loading channel of the Y-junction and into the metered dose pump.

FIG. 13A shows a cross section of a diffuser 12170 as seated within the device 1200, according to an additional embodiment. The diffuser 12170 may be an embodiment of the diffuser 170. In this implementation of the device 1200, the diffuser 12170 is annular. As shown in FIG. 13A, the diffuser 12170 sits on a shelf 1305 inside a bore 1310 of the Y-junction 12120, and the dose chamber 12150 is inserted into the bore 1310 of the Y-junction 12120. The diffuser 12170 is seated between the shelf of the bore of the Y-junction 12120 and a bottom face of the dose chamber 12150, sealing against both of those surfaces. The diffuser 12170 may further be sealed along its inner diameter to the Y-junction 12120. In this configuration, the diffuser 12170 creates an interference seal along its inner diameter, its upper face, and its lower outer edge (in contact with the shelf 1305). This configuration may allow expansion of the diffuser 12170, for example, as propellant flows through the diffuser 12170 due to changes in temperature or as a result of device assembly. Sealing the diffuser 12170 along its inner diameter may improve the consistency and/or quality of the seal and/or performance of the diffuser 12170 relative to sealing the diffuser 12170 along its top and bottom faces in a compression fit, which could compress the diffusion path within (the path along which propellant travels and is diffused). In this configuration, variations in the manufacturing of the diffuser 12170 may be less likely to affect the performance of the diffuser 12170. For example, the tolerances of the outer diameter of the diffuser 12170 may not need to be as precisely controlled to prevent bending of the diffuser 12170 such that flatness of the diffuser 12170 is maintained to ensure a proper compression fit along its faces. In some instances, the interference seal may or may not be liquid or gas tight.

FIG. 13B shows an exploded view of the dose chamber 12150 and the Y-junction 12120, according to an additional embodiment. FIG. 13B illustrates the bore 1310 and the shelf 1305 of the Y-junction 12120. The dose chamber 12150 may include a chamfer 1315 around an outer edge of its bottom face such that the dose chamber 12150 may be easily inserted into the bore 1310. In alternate embodiments, the configuration of the dose chamber 12150 and Y-junction 12120 may be reversed such that the dose chamber 12150 includes a bore into which a diffuser and an end of the Y-junction 12120 is inserted.

FIG. 14 illustrates the nose cone 12200, according to an additional embodiment. The nose cone 12200 may be an embodiment of the nose cone 200. As previously described, the external geometries of the nose cone 12200 assist in providing proper alignment of the device 1200 during insertion into the nose. As shown in FIG. 14, the nose cone 12200 comprises an opening 1405 that aligns with the nozzle (not shown). The dose chamber 12150 (not shown in this view) may be positioned between two bosses 1410a, 1410b that maintain the alignment of the dose chamber 12150 and the nozzle within the nose cone 12200. In the embodiment of FIG. 14, the nose cone 12200 is integrated into the design of the clamshells. The nose cone 12200 and the clamshells may be molded together during manufacturing, decreasing the overall part count of the device 1200 and enabling easy assembly of the device 1200.

### 5.5.4. Device for Administration of Dry Powder Composition

The powder composition can be intranasally administered utilizing any conventional device in the field. For example, the composition can be administered utilizing a dispenser, for example a single use dispenser or a multi-use dispenser. In certain embodiments the powder composition is intranasally administered using a device such as, for example, a device as described in US 2011/0045088 or in WO 2012/105236, each of which is incorporated herein by reference in its entirety. In specific embodiments, the device used to administer the powder composition is a Fit-lizer™ (SNBL, LTD) intranasally dispenser device.

In some embodiments, the device used to intranasally administer the powder composition comprises a nozzle having an upstream end and a downstream end adapted to allow positioning of at least a portion of the nozzle into a nostril of a subject; a reservoir comprising a single dose of a powdered therapeutic formulation, the reservoir having an upstream end and a downstream end, and disposed within the nozzle; a valve having an upstream end and a downstream end, wherein the valve is adapted to occupy a first position and a second position in the device, and wherein the valve is adapted to cause diffusion of the powdered therapeutic formulation when the device is activated; and an air source operably linked to the upstream end of a valve, wherein the device is a single-use device.

In certain embodiments, the dry powder composition is delivered by an intranasal dispenser device. In some embodiments, the device comprises an air source that is adapted to be engaged by a user to force air from an air source through a valve assembly into a reservoir and out of a nozzle. In typical embodiments, the device is operated by applying compressive force to a pump. In some embodiments, the pump comprises a manual air pump.

The powder composition can be delivered by pulmonary administration utilizing any conventional techniques is the field, for example, a device as described in U.S. Pat. No. 8,119,639, which is incorporated herein by reference in its entirety. In some embodiments, the method of administration is by pulmonary inhalation using aerosols, dry powder inhalers, nebulizers, vaporizers, pressurized metered dose inhalers (pMDIs) and the like. In some embodiments, a pMDI such as a breath activated metered dose inhaler (for example, TEMPO™ Inhaler from Map Pharmaceuticals, Mountain View, Calif.) is used to administer DHE.

In some embodiments, the device comprises a breath activated pressurized metered dose inhaler. In some embodiments, the breath activated pressurized metered dose inhaler comprises a plume control feature. In some embodiments, the breath activated pressurized metered dose inhaler comprises a vortexing chamber.

In some embodiments, the breath actuated pressurized metered dose inhaler contains a suspension of the DHE or salt thereof in hydrofluoroalkane propellant. In some embodiments, the hydrofluoroalkane propellant is HFA134a

In some embodiments, the breath actuated pressurized metered dose inhaler contains a suspension of the DHE or salt thereof in a hydrofluoroalkane propellant blend.

### 5.6. Kits

In another aspect, kits are provided for treating migraine headaches in a subject to achieve sustained reduced frequency of migraine headaches by administering a pharmaceutical composition via the respiratory system. The subject may have migraine headaches with or without aura.

The kit comprises a vial and a device. In some embodiments, the vial is sealed, and sealably contains at least one effective dose of a liquid pharmaceutical composition comprising dihydroergotamine (DHE) or salt thereof. The vial can be configured to be attachable to the device. The device can be reciprocally configured to receive the vial. Upon attachment of the vial to the device by the user, the device can become a manually actuated, propellant-driven, metered-dose intranasal administration device capable of providing, after intranasal administration of a dose of liquid pharmaceutical composition, (a) a mean peak plasma DHE concentration (Cₘₐₓ) of at least 750 pg/ml, (b) with a mean time to Cₘₐₓ (Tₘₐₓ) of DHE of less than 45 minutes, and (c) a mean plasma AUC_{0-inf} of DHE of at least 2000 pg*hr/ml.

In typical embodiments, upon attachment of the vial to the device, the device becomes a manually actuated, propellant-driven, metered-dose intranasal administration device as described in Section 4.5 above. In some embodiments, upon attachment of the vial to the device, the device becomes a manually actuated, propellant-driven, metered-dose intranasal administration device as particularly described in Section 4.5 above. In some embodiments, the propellant-containing canister is a pressurized canister that is sealed within the device and is not accessible to the user.

In various embodiments, the vial is a sealed glass vial. In some embodiments, the vial is a 3.5-mL amber sealed glass vial.

In typical embodiments, the liquid pharmaceutical composition that is sealably contained within the vial is a liquid pharmaceutical composition as described in Section 5.3.2 above. In some embodiments, the vial comprises a liquid pharmaceutical composition having the following composition: a clear, colorless to faintly yellow solution in an amber glass vial containing:

| | |
|---|---|
| dihydroergotamine mesylate, USP | 4.0 mg |
| caffeine, anhydrous, USP | 10.0 mg |
| dextrose, anhydrous, USP | 50.0 mg |
| carbon dioxide, USP | qs |
| purified water, USP | qs 1.0 mL. |

The vial contains liquid pharmaceutical composition in an amount sufficient for at least one total dose of DHE, or salt thereof, to be delivered by the device, in a single undivided or a plurality of divided doses. In particular embodiments, the vial contains liquid pharmaceutical composition in an amount sufficient for at most one total dose of DHE, or salt thereof, to be delivered by the device, in a single undivided or a plurality of divided doses.

In typical embodiments, the propellant canister within the device that is co-packaged with the vial in the kit contains pressurized propellant in an amount sufficient for optional priming of the device followed by delivery of at least one total dose of DHE, or salt thereof, to be delivered by the device either in a single undivided or a plurality of divided doses. In particular embodiments, the propellant canister contains pressurized propellant in an amount sufficient for optional priming of the device followed by delivery of at most one total dose of DHE, or salt thereof, to be delivered by the device, in a single undivided or a plurality of divided doses.

### 6. EXPERIMENTAL EXAMPLES

The invention is further described through reference to the following experimental examples. These examples are provided for purposes of illustration only, and are not intended to be limiting.

### 6.1. Example 1: Reproducibility of Dose Delivery

Table 2 provides experimental data on one implementation of the in-line device described in Section 5.5.3 above. As used in Table 2, “dose” refers to a volume delivered in a single device actuation.

TABLE 2

| | Dose Volume [μL] | | | | | |
|---|---|---|---|---|---|---|
| Shot # | Device 1 | Device 2 | Device 3 | Device 4 | Device 5 | Device 6 |
| 1 | 190.6 | 193.7 | 185.3 | 199.2 | 199.2 | 145.1 |
| 2 | 181.4 | 205.5 | 178.9 | 167.7 | 167.7 | 141.7 |
| 3 | 183.1 | 188.5 | 173.3 | 165.6 | 165.6 | 138.5 |
| 4 | 183.2 | 193.3 | 145.8 | 164.6 | 164.6 | 136.6 |
| 5 | 183.3 | 201.5 | 200.7 | 162.0 | 162.0 | 142.1 |
| 6 | 185.8 | 207.7 | 166.3 | 179.4 | 179.4 | 138.9 |
| 7 | 184.3 | 195.1 | 180.3 | 164.8 | 164.8 | 140.9 |
| 8 | 183.3 | 205.4 | 175.3 | 164.9 | 164.9 | 142.0 |
| 9 | 180.5 | 178.1 | 172.0 | 164.1 | 164.1 | 141.8 |
| 10 | 179.7 | 204.0 | 178.0 | 170.6 | 170.6 | 143.9 |
| Mean | 183.5 | 197.3 | 175.6 | 170.3 | 170.3 | 141.2 |
| StDev | 3.1 | 9.3 | 14.0 | 11.3 | 11.3 | 2.5 |
| Min | 179.7 | 178.1 | 145.8 | 162.0 | 162.0 | 136.6 |
| Max | 190.6 | 207.7 | 200.7 | 199.2 | 199.2 | 145.1 |

| | |
|---|---|
| 185 uL + 10% | 203.5 |
| 185 uL − 10% | 166.5 |
| 185 uL + 15% | 212.8 |
| 185 uL − 15% | 157.3 |

### 6.2. Example 2: Phase I Clinical Trial

A Phase I clinical trial was conducted to compare the bioavailability of dihydroergotamine (DHE) mesylate following (i) single divided dose intranasal administration of INP104, a drug-device combination employing a Precision Olfactory Delivery (POD®) Device (Impel NeuroPharma, Seattle); (ii) intranasal administration of Migranal® Nasal Spray (Valeant Pharmaceuticals); and (iii) intravenous injection with D.H.E. 45® (Valeant Pharmaceuticals) in healthy adult subjects.

### 6.2.1. Study Design

The study was a three-period, three-way, randomized, open-label, single-dose, cross-over, comparative bioavailability study. Treatment assignment was randomized in a three-treatment, three-period balanced crossover study of six sequences shown below, with a 7-day washout between treatments:

TABLE 3

| | Treatment | | |
|---|---|---|---|
| Sequence | 1 | 2 | 3 |
| 1 | A | B | C |
| 2 | B | C | A |
| 3 | C | A | B |
| 4 | A | C | B |
| 5 | B | A | C |
| 6 | C | B | A |

A = 1.45 mg INP104
B = 1.0 mg D.H.E. 45, IV
C = 2 mg Migranal ® Nasal Spray.

Subjects all received 10 mg IV metoclopramide 5-10 minutes prior to each treatment.

Thirty-eight subjects (approximately equal numbers of men and women) were enrolled and randomized into the study. A total of 36 (94.7%) subjects received at least 1 dose of INP104 (investigational product) and were included in the Safety Population. Overall, 29 subjects received all 3 scheduled doses of DHE (Migranal, INP104, and DHE 45); of these, 27 subjects had sufficient data for calculation of all PK parameters for each of the 3 doses and were included in the primary PK Population.

INP104 was self-administered using the 1123 POD™ Device (Impel NeuroPharma, Seattle). The dose of DHE mesylate was divided, with one spray in each nostril delivering a total target dose of 1.45 mg DHE mesylate.

The 1123 POD Device is a handheld, manually actuated, propellant-driven, metered-dose administration device intended to deliver a drug formulation to the nasal cavity as illustrated in FIGS. 12-14. Drug delivery to the nasal cavity via the 1123 POD Device is driven by hydrofluoroalkane-134a (HFA) propellant. The 1123 POD Device functions as an intranasal delivery device; the HFA propellant in the 1123 POD Device is not intended to deliver drug to the lungs and does not contact the DHE formulation until the time of delivery.

The INP104 drug component, DHE DP, is a 3.5-mL amber glass vial filled with DHE mesylate 4 mg/mL. The formulation is identical to that in the Migranal® Nasal Spray device: a clear, colorless to faintly yellow solution in an amber glass vial containing:

| | |
|---|---|
| dihydroergotamine mesylate, USP | 4.0 mg |
| caffeine, anhydrous, USP | 10.0 mg |
| dextrose, anhydrous, USP | 50.0 mg |
| carbon dioxide, USP | qs |
| purified water, USP | qs 1.0 mL. |

The DHE DP vial attaches to the 1123 POD Device. The 1123 POD Device may have a nominal output between 175 μL/actuation pump and 205 μL/actuation pump (inclusive). In some embodiments, the 1123 POD Device has a nominal output that is about 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, or 205 μL/actuation pump.

A single manual actuation of the device by the user results in the operation of the metering pump to fill the dose chamber with the DHE formulation and subsequent, but almost instantaneous, activation of the propellant canister to expel the formulation through the nozzle, as a spray, resulting in delivery to the nasal cavity of the user. The device is designed to be disposed of after successful single divided-dose drug delivery (1 spray per nostril). Actuation of the 1123 POD Device releases approximately 63 μL of HFA-134a propellant, similar to HFA exposure from metered-dose inhalers.

D.H.E. 45® (Valeant Pharmaceuticals (now Bausch), NDA 005929) was administered in a volume of 1 mL intravenously over 1 minute.

Migranal® Nasal Spray (2 mg) (Valeant Pharmaceuticals (now Bausch), NDA 20148) was self-administered with equal dosing to both nostrils. In accordance with the product label, one spray (0.5 mg) was administered in each nostril initially, followed by an additional spray (0.5 mg) in each nostril 15 minutes later.

### 6.2.2. Pharmacokinetic Assessments

### Sampling and Processing

Blood samples for PK analysis were obtained, according to the clinical trial site's standard operating procedures (SOPs), within 15 minutes prior to dosing and at 5, 10, 20, 30, 40 and 50 minutes, and 1, 1.25, 1.5, 1.75, 2, 3, 4, 8, 12, 24, 36 and 48 hours after dosing. For the Migranal® Nasal Spray dose, the PK sampling timeclock was started following administration of the first dose of Migranal® Nasal Spray.

### Pharmacokinetic Analysis

Individual DHE and 8′-OH-DHE plasma concentration data were listed for each individual and summarized by nominal sampling time-point and administration method with descriptive statistics (sample size [N], arithmetic mean, standard deviation [SD], median, minimum, maximum and geometric mean). Individual and mean DHE and 8′-OH-DHE plasma concentration-time profiles for each administration method were also graphed.

Pharmacokinetic parameters were computed from the individual plasma DHE and 8′-OH-DHE concentrations using a non-compartmental approach. Appropriate validated PK software (e.g., Phoenix WinNonlin v6.3) was used. The parameters that were determined and their definitions are provided in Table 4 below.

TABLE 4

| | |
|---|---|
| Cₘₐₓ | Maximum observed drug concentration. |
| Tₘₐₓ | Time to maximum observed drug concentration. If the maximum value |
| | occurs at more than one time-point, Tₘₐₓ is defined as the first time point |
| | with this value. |
| AUC₀₋ₜ | Area under the drug concentration-time curve, calculated using linear-up |
| | log-down trapezoidal summation from time zero to the time of the last |
| | Measurable concentration. |
| kₑₗ | Apparent terminal elimination rate constant, calculated by linear regression |
| | of the terminal linear portion of the log concentration vs. time curve. |
| AUC_{0-inf} | Area under the drug concentration-time curve from time zero to infinity, |
| | calculated as AUC₀₋ₜ + Ct/kₑₗ. |
| t_{1/2} | Apparent elimination half-life, calculated as ln(2)/kₑₗ. |
| CL/F | Apparent clearance calculated as Dose/AUC_{0-inf}. |
| (CL for i.v.) | |
| Vz/F | Apparent volume of distribution at the terminal phase, calculated as Dose/(ₑₗ |
| (Vz for i.v.) | * AUC_{0-inf}). |

### Statistical Methods for Pharmacokinetic Analyses

PK parameters were summarized by administration method using descriptive statistics (arithmetic means, SD, coefficients of variation [CV], sample size [N] minimum, maximum, median and geometric mean). Geometric mean was calculated for AUC₀₋₄, AUC_{0-inf}, and Cₘₐₓ.

No value for kₑ₁, t_{1/2}, AUC_{0-inf}, CL/F, Vz/F, as appropriate, were reported for cases that did not exhibit a terminal log-linear phase in the concentration versus time profile or did not contain sufficient data during this phase for parameter estimation.

### Statistical Analysis

A comparative bioavailability assessment was undertaken to demonstrate (i) that the lower 90% confidence interval of the DHE after INP104 to DHE after Migranal Nasal Spray geometric mean ratios for Cₘₐₓ and AUC (AUC₀₋₄, AUC_{0-inf}) is not less than 80%, and (ii) the upper 90% confidence interval of the DHE after INP104 to D.H.E. 45 Injection (IV) geometric mean ratios for Cₘₐₓ and AUC (AUC₀₋₄, AUC_{0-inf}) not greater than 125%—i.e., to demonstrate that exposure is equal to or greater than 80% and equal to or less than 125% range observed between Migranal Nasal Spray and D.H.E. 45 Injection (IV), respectively.

For each comparator (Migranal Nasal Spray and D.H.E. 45 Injection (IV)), the following analysis methods were performed independently. Analysis of variance (ANOVA) with effects for sequence, subject nested within sequence, period, and treatment were performed on the ln-transformed DHE and 8′OH-DHE AUC₀₋₄, AUC_{0-inf} and Cₘₐₓ. Each ANOVA included calculation of least squares mean (LSM), the difference between administration method LSM, and the standard error associated with the difference.

Only subjects who had completed all three treatments and had sufficient PK sample collection to generate the key PK parameters (AUC₀₋₄, AUC_{0-inf} and Cₘₐₓ) for each administration method were included in the ANOVA analysis.

Ratios of geometric means were calculated using the exponentiation of the difference between treatment LSM from the analyses on the ln-transformed AUC₀₋₄, AUC_{0-inf} and Cₘₐₓ. These ratios were expressed as a percentage relative to the reference (comparator) treatment, i.e. INP104 [test]/Comparator [reference]. Consistent with the two one-sided tests for bioequivalence, 90% confidence intervals were obtained for the ratio of the geometric means for AUC₀₋₄, AUC_{0-inf} and Cₘₐₓ.

### 6.2.3. Results: DHE and 8′OH-DHE Pharmacokinetics

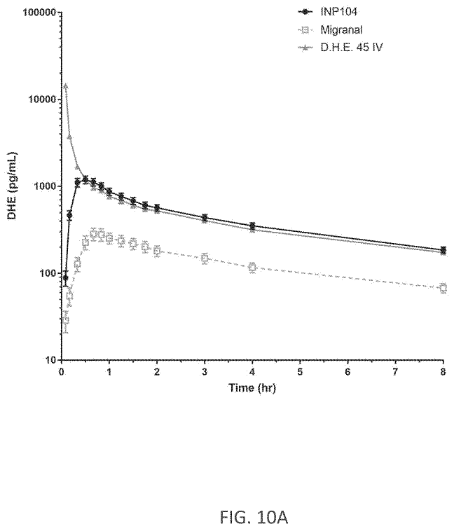

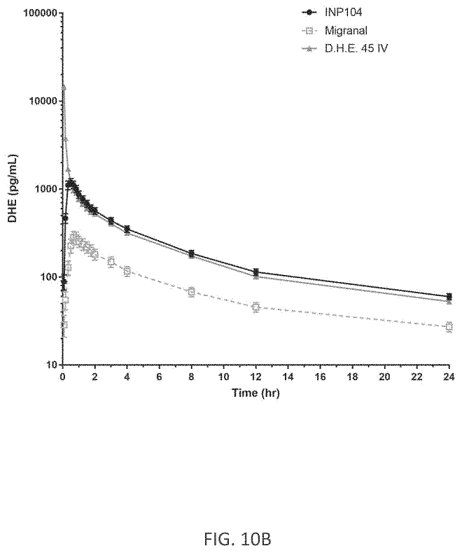

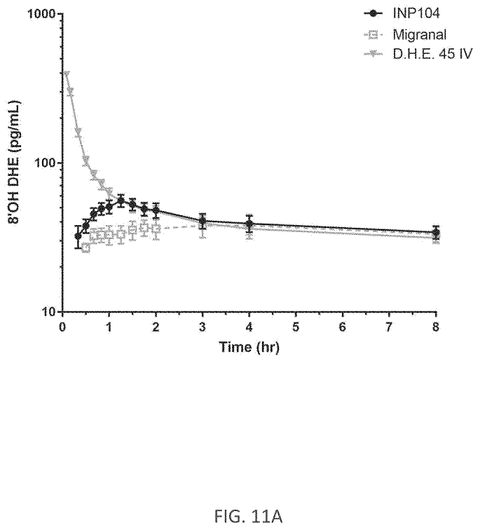

The time course of plasma DHE concentrations is plotted in FIGS. 10A and 10B, and initial summary statistics are provided in Tables 5A and 5B below.

### Safety Population

Plasma dihydroergotamine PK parameters were derived for all subjects in the Safety Population (i.e., all subjects who received at least 1 dose of any DHE product). A summary of dihydroergotamine plasma PK parameters for the Safety Population is presented in Table 5A.

Following administration of D.H.E. 45, mean Cₘₐₓ was 14190 pg/mL, compared with 1301 pg/mL and 299.6 pg/mL for INP104 and Migranal, respectively. Attainment of peak dihydroergotamine plasma concentrations was fastest following D.H.E. 45 administration with a median Tₘₐₓ of 0.083 hours (5 min post-dose, the first sampling time point), followed by INP104 at 0.5 hours and Migranal at 0.783 hours. The median t_{1/2} was similar for D.H.E. 45 (13.717 hours), INP104 (11.109 hours) and Migranal (10.732 hours). The mean AUC₀₋ₗₐₛₜ was 7091 h*pg/mL, 5930 h*pg/mL, and 1834 h*pg/mL for D.H.E. 45, INP104, and Migranal, respectively. The mean AUC₀₋₂ₕ was 3022 h*pg/mL, 1603 h*pg/mL, and 387.5 h*pg/mL for D.H.E. 45, INP104, and Migranal, respectively. The mean km was similar for D.H.E. 45, INP104, and Migranal (0.05184 l/h, 0.06173 l/h, and 0.07411 l/h, respectively). The mean CL was, as expected, lowest for D.H.E. 45 at 117.9 L/h while the CL/F was approximately 2-fold higher at 267.0 L/h for INP104 and approximately 11-fold higher for Migranal at 1293 L/h. Similarly, the mean Vz (or Vz/F) was lowest for D.H.E. 45 at 2432 L, followed by INP104 at 4314 L and Migranal at 16860 L.

These differences were likely to be largely due to differences in bioavailability between nasal administrations and IV administration, with reduced bioavailability resulting in relative increases in CL/F and Vz/F values rather than true differences in actual systemic clearance and distribution. Mean Cₘₐₓ following D.H.E. 45 administration was greater and occurred earlier than in the nasal IPs, and mean Cₘₐₓ following INP104 administration was 4-fold higher than observed following Migranal administration.

TABLE 5A
Plasma Dihydroergotamine Pharmacokinetic Parameters (Safety Population)

| | | Cmax | Tmax | AUC₀₋ₗₐₛₜ | AUC_{0-2 h} | AUC_{0-inf} |
|---|---|---|---|---|---|---|
| Treatment | Statistic | (pg/mL) | (h) | (h*pg/mL) | (h*pg/mL) | (h*pg/mL) |
| 1.45 mg | N | 31 | 31 | 31 | 31 | 31 |
| INP104 | Mean | 1301 | 0.549 | 5930 | 1603 | 6275 |
| | SD | 668.33 | 0.3111 | 2520.8 | 783.26 | 2621.2 |
| | CV % | 51.4 | 56.7 | 42.5 | 48.9 | 41.8 |
| | Median | 1240 | 0.500 | 5800 | 1615 | 6082 |
| | Minimum | 270 | 0.33 | 737 | 331 | 969 |
| | Maximum | 2660 | 2.05 | 9920 | 3120 | 10400 |
| | Geometric | 1119 | | 5224 | 1389 | 5583 |
| | Mean | | | | | |
| | Geometric | 64.8 | | 63.4 | 63.8 | 59.1 |
| | CV (%) | | | | | |
| 1 mg | N | 31 | 31 | 31 | 31 | 31 |
| D.H.E. 45 | Mean | 14190 | 0.107 | 7091 | 3022 | 7490 |
| | SD | 5247.9 | 0.1257 | 1224.9 | 529.61 | 1245.5 |
| | CV (%) | 37.0 | 117.5 | 17.3 | 17.5 | 16.6 |
| | Median | 13900 | 0.083 | 7128 | 2982 | 7564 |
| | Minimum | 1560 | 0.07 | 4820 | 2250 | 5250 |
| | Maximum | 23200 | 0.78 | 9930 | 4000 | 10200 |
| | Geometric | 12910 | | 6990 | 2977 | 7390 |
| | Mean | | | | | |
| | Geometric | 54.8 | | 17.4 | 17.8 | 16.8 |
| | CV (%) | | | | | |
| 2 mg | N | 34 | 34 | 34 | 34 | 31 |
| Migranal | Mean | 299.6 | 1.021 | 1834 | 387.5 | 2199 |
| | SD | 275.14 | 0.6048 | 1589.9 | 334.15 | 1643.1 |
| | CV (%) | 91.8 | 59.3 | 86.7 | 86.2 | 74.7 |
| | Median | 206.0 | 0.783 | 1383 | 270.3 | 1648 |
| | Minimum | 25.4 | 0.5 | 132 | 30.9 | 436 |
| | Maximum | 1190 | 3.08 | 6680 | 1460 | 7120 |
| | Geometric | 198.4 | | 1248 | 263.8 | 1704 |
| | Mean | | | | | |
| | Geometric | 126.0 | | 120.7 | 121.9 | 84.6 |
| | CV (%) | | | | | |

| | | kₑₗ | t_{1/2} | CL/F | Vz/F |
|---|---|---|---|---|---|
| Treatment | Statistic | (1/h) | (h) | (L/h) | (L) |
| 1.45 mg | N | 31 | 31 | 31 | 31 |
| INP104 | Mean | 0.06173 | 11.777 | 267.0 | 4314 |
| | SD | 0.013228 | 2.8229 | 224.29 | 2858.5 |
| | CV % | 21.4 | 24.0 | 84.0 | 66.3 |
| | Median | 0.06240 | 11.109 | 203.9 | 3072 |
| | Minimum | 0.0312 | 7.36 | 119 | 2040 |
| | Maximum | 0.0942 | 22.22 | 1280 | 13600 |
| | Geometric | 0.06033 | | 222.1 | 3681 |
| | Mean | | | | |
| | Geometric | 22.4 | | 59.1 | 57.7 |
| | CV (%) | | | | |
| 1 mg | N | 31 | 31 | 31 | 31 |
| D.H.E. 45 | Mean | 0.05184 | 14.201 | 117.9 | 2432 |
| | SD | 0.013283 | 3.4926 | 19.665 | 771.33 |
| | CV (%) | 25.6 | 24.6 | 16.7 | 31.7 |
| | Median | 0.05053 | 13.717 | 113.7 | 2327 |
| | Minimum | 0.0326 | 8.06 | 84.4 | 1150 |
| | Maximum | 0.0860 | 21.29 | 164 | 4070 |
| | Geometric | 0.05028 | | 116.4 | 2314 |
| | Mean | | | | |
| | Geometric | 25.4 | | 16.8 | 33.1 |
| | CV (%) | | | | |
| 2 mg | N | 31 | 31 | 31 | 31 |
| Migranal | Mean | 0.07411 | 10.417 | 1293 | 16860 |
| | SD | 0.027739 | 3.0921 | 917.08 | 9380.4 |
| | CV (%) | 37.4 | 29.7 | 70.9 | 55.6 |
| | Median | 0.06459 | 10.732 | 1044 | 15900 |
| | Minimum | 0.0422 | 4.99 | 242 | 4520 |
| | Maximum | 0.139 | 16.41 | 3950 | 42400 |
| | Geometric | 0.06993 | | 1009 | 14440 |
| | Mean | | | | |
| | Geometric | 34.4 | | 84.6 | 63.4 |
| | CV (%) | | | | |

Abbreviations:
BA = bioavailability; CV = coefficient of variation; SD = standard deviation.
Notes:
Pharmacokinetic parameters determined using Phoenix WinNonLin v6.3.
Pharmacokinetic parameters for Subject 100-025 after 1 mg D.H.E. 45 (Period 2) were not derived due to insufficient sampling during the peak concentration phase (5- and 10-minute samples were collected at the same clock time as the 20-minute sample).
For 1 mg D.H.E. 45, BA was assumed to be 1.00. As such CL/F = CL and Vz/F = Vz.

### PK Population

Plasma dihydroergotamine PK parameters were also derived for all subjects in the PK Population (i.e., all subjects who received all three DHE products—Migranal, D.H.E. 45 and INP104—and provided a sufficient number of blood samples where data were sufficient for parameter estimation using non-compartmental analysis). Plasma dihydroergotamine PK parameters for the PK Population are summarized in Table 5B.

Following administration of D.H.E. 45, mean Cₘₐₓ was 14620 pg/mL, compared with 1281 pg/mL and 328.5 pg/mL for INP104 and Migranal, respectively. Attainment of peak dihydroergotamine plasma concentrations was fastest following D.H.E. 45 administration with a median Tₘₐₓ of 0.083 hours followed by INP104 at 0.5 hours, and Migranal at 0.667 hours. The median t_{1/2} was similar for D.H.E. 45 (13.827 hours), INP104 (11.842 hours), and Migranal (10.732 hours). For each of the test and reference products, plasma PK sampling covered more than 3 terminal elimination half-lives of dihydroergotamine. The mean AUC₀₋ₗₐₛₜ were 6967 h*pg/mL, 5807 h*pg/mL, and 1999 h*pg/mL for D.H.E. 45, INP104, and Migranal, respectively. The mean AUC₀₋₂ₕ was 3019 h*pg/mL, 1595 h*pg/mL, and 428.7 h*pg/mL for D.H.E. 45, INP104, and Migranal, respectively. The mean AUC_{0-inf} were 7381 h*pg/mL, 6153 h*pg/mL, and 2208 h*pg/mL for D.H.E. 45, INP104, and Migranal, respectively. On average, ≥90% of the AUC was captured during the sampling period when comparing mean AUC₀₋ₗₐₛₜ to AUC_{0-inf} for each product. For INP104, the AUC percent extrapolated was >10% for 3 subjects, with 1 of those 3 subjects having AUC percent extrapolated >20%. For D.H.E. 45 and Migranal, the AUC percent extrapolated >10% occurred for zero and 15 subjects, respectively. For Migranal, 4 of those 15 subjects had AUC percent extrapolated >20%. The mean kₑ₁ was similar for D.H.E. 45, INP104, and Migranal (0.05086 L/h, 0.06088 L/h, and 0.07187 L/h, respectively). The mean CL was lowest for D.H.E. 45 at 119.3 L/h. INP104 was approximately 2-fold higher at 279.5 L/h, and Migranal was approximately 10-fold higher at 1208 L/h. Similarly, the mean Vz was lowest for D.H.E. 45 at 2505 L, followed by INP104 at 4546 L, and Migranal at 16370 L.

In the PK Population, the PK parameters were generally similar to those from the Safety Population for each treatment; however, due to the exclusion of some subjects from the PK population, there was a slight decrease in AUC₀₋ₗₐₛₜ and AUC_{0-inf} for INP104 and D.H.E. 45 in the PK Population, whereas there was a slight increase in these parameters and in Cₘₐₓ for Migranal in the PK Population.

TABLE 5B
Plasma Dihydroergotamine Pharmacokinetic Parameters (PK Population)

| | | Cmax | Tmax | AUC₀₋ₗₐₛₜ | AUC_{0-2 h} | AUC_{0-inf} |
|---|---|---|---|---|---|---|
| Treatment | Statistic | (pg/mL) | (h) | (h*pg/mL) | (h*pg/mL) | (h*pg/mL) |
| 1.45 mg | N | 27 | 27 | 27 | 27 | 27 |
| INP104 | Mean | 1281 | 0.511 | 5807 | 1595 | 6153 |
| | SD | 682.11 | 0.1409 | 2623.4 | 800.90 | 2721.4 |
| | CV % | 53.3 | 27.6 | 45.2 | 50.2 | 44.2 |
| | Median | 1240 | 0.500 | 5800 | 1615 | 6082 |
| | Minimum | 270 | 0.33 | 737 | 331 | 969 |
| | Maximum | 2660 | 0.78 | 9920 | 3120 | 10400 |
| | Geometric | 1093 | | 5043 | 1372 | 5407 |
| | Mean | | | | | |
| | Geometric | 66.7 | | 67.3 | 65.7 | 62.6 |
| | CV (%) | | | | | |
| 1 mg | N | 27 | 27 | 27 | 27 | 27 |
| D.H.E. 45 | Mean | 14620 | 0.085 | 6967 | 3019 | 7381 |
| | SD | 4906.4 | 0.0064 | 1094.9 | 513.44 | 1138.5 |
| | CV (%) | 33.6 | 7.6 | 15.7 | 17.0 | 15.4 |
| | Median | 13900 | 0.083 | 7128 | 2982 | 7564 |
| | Minimum | 6630 | 0.07 | 4820 | 2250 | 5250 |
| | Maximum | 23200 | 0.1 | 9050 | 4000 | 9530 |
| | Geometric | 13830 | | 6883 | 2976 | 7295 |
| | Mean | | | | | |
| | Geometric | 35.4 | | 16.1 | 17.4 | 15.7 |
| | CV (%) | | | | | |
| 2 mg | N | 27 | 27 | 27 | 27 | 27 |
| Migranal | Mean | 328.5 | 0.837 | 1999 | 428.7 | 2208 |
| | SD | 260.70 | 0.3014 | 1433.1 | 317.81 | 1487.8 |
| | CV (%) | 79.4 | 36.0 | 71.7 | 74.1 | 67.4 |
| | Median | 283.0 | 0.667 | 1689 | 391.7 | 1864 |
| | Minimum | 50.2 | 0.5 | 343 | 77.6 | 436 |
| | Maximum | 1190 | 1.8 | 6680 | 1460 | 7120 |
| | Geometric | 248.3 | | 1554 | 332.7 | 1784 |
| | Mean | | | | | |
| | Geometric | 91.4 | | 87.1 | 86.1 | 77.2 |
| | CV (%) | | | | | |

| | | kₑₗ | t_{1/2} | CL/F | Vz/F |
|---|---|---|---|---|---|
| Treatment | Statistic | (1/h) | (h) | (L/h) | (L) |
| 1.45 mg | N | 27 | 27 | 27 | 27 |
| INP104 | Mean | 0.06088 | 11.934 | 279.5 | 4546 |
| | SD | 0.012945 | 2.8646 | 237.91 | 2993.7 |
| | CV % | 21.3 | 24.0 | 85.1 | 65.9 |
| | Median | 0.05853 | 11.842 | 203.9 | 3108 |
| | Minimum | 0.0312 | 7.36 | 119 | 2040 |
| | Maximum | 0.0942 | 22.22 | 1280 | 13600 |
| | Geometric | 0.05952 | | 229.3 | 3853 |
| | Mean | | | | |
| | Geometric | 22.3 | | 62.6 | 60.2 |
| | CV (%) | | | | |
| 1 mg | N | 27 | 27 | 27 | 27 |
| D.H.E. 45 | Mean | 0.05086 | 14.511 | 119.3 | 2505 |
| | SD | 0.013535 | 3.5893 | 18.917 | 769.38 |
| | CV (%) | 26.6 | 24.7 | 15.9 | 30.7 |
| | Median | 0.05013 | 13.827 | 113.7 | 2327 |
| | Minimum | 0.0326 | 8.06 | 90.2 | 1310 |
| | Maximum | 0.0860 | 21.29 | 164 | 4070 |
| | Geometric | 0.04925 | | 117.9 | 2393 |
| | Mean | | | | |
| | Geometric | 26.0 | | 15.7 | 31.6 |
| | CV (%) | | | | |
| 2 mg | N | 27 | 27 | 27 | 27 |
| Migranal | Mean | 0.07187 | 10.645 | 1208 | 16370 |
| | SD | 0.026173 | 3.0211 | 869.26 | 9121.5 |
| | CV (%) | 36.4 | 28.4 | 72.0 | 55.7 |
| | Median | 0.06459 | 10.732 | 922.6 | 15550 |
| | Minimum | 0.0422 | 4.99 | 242 | 4520 |
| | Maximum | 0.139 | 16.41 | 3950 | 42400 |
| | Geometric | 0.06812 | | 964.3 | 14150 |
| | Mean | | | | |
| | Geometric | 32.9 | | 77.2 | 60.1 |
| | CV (%) | | | | |

Abbreviations:
BA = bioavailability; CV = coefficient of variation; PK = pharmacokinetic SD = standard deviation.
Notes:
Pharmacokinetic parameters determined using Phoenix WinNonLin v6.3.
Pharmacokinetic parameters for Subject 100-025 after 1 mg D.H.E. 45 (Period 2) were not derived due to insufficient sampling during the peak concentration phase (5- and 10-minute samples were collected at the same clock time as the 20-minute sample).
For 1 mg D.H.E. 45, BA was assumed to be 1.00. As such CL/F = CL and Vz/F = Vz.

As compared to Migranal® Nasal Spray, INP104 provides nearly 3-fold higher mean systemic drug exposure, with an AUC_{0-inf} of 5,407 pg*hr/ml as compared to 1,784 pg*hr/ml for Migranal®. INP104 also provides nearly 4-fold higher mean maximal plasma concentration, with a Cₘₐₓ of 1,093 pg/ml as compared to 248.3 pg/ml for Migranal®. Maximal DHE plasma concentration is reached faster with INP104, with a mean Tₘₐₓ of 34 minutes versus 55 minutes for Migranal®. The higher systemic drug exposure and higher maximal plasma concentration were achieved with a lower administered dose of the identical formulation of DHE mesylate, 1.45 mg for INP104 versus 2.0 mg for Migranal®, and without requiring a 15-minute wait between administration of divided sub-doses, as required for Migranal®.

In addition, systemic delivery of DHE was more consistent with INP104 than with Migranal®, with lower variation observed across subjects for both AUC_{0-inf} and Cₘₐₓ parameters (see Table 5A and Table 5B above for coefficients of variation).

Although bolus intravenous administration of 1 mg DHE mesylate provided greater than 10-fold higher Cₘₐₓ than 1.45 mg DHE mesylate administered intranasally by INP104, the high Cₘₐₓ achieved with intravenous administration is known to be correlated with adverse events (“AE”s), specifically nausea, and IV DHE mesylate (D.H.E. 45) is most commonly administered with an anti-emetic. Within 20-30 minutes following administration, DHE plasma concentrations achieved through INP104 intranasal administration were essentially indistinguishable from concentrations achieved by intravenous administration. Thus, despite a greater than 10-fold higher Cₘₐₓ, bolus intravenous administration of 1 mg DHE mesylate provided less than 2-fold greater systemic drug delivery, measured as AUC_{0-inf}, as compared to INP104 intranasal delivery.

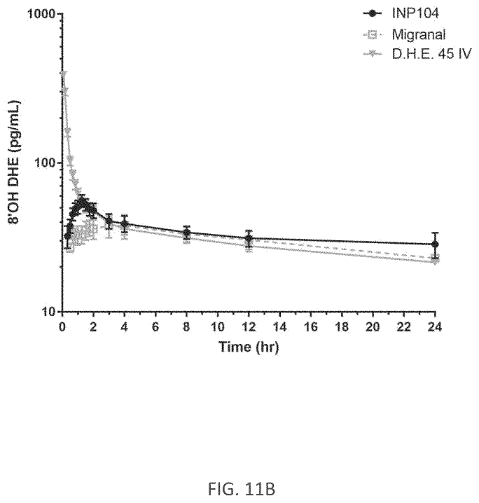

The 8′OH-DHE metabolite of DHE is known to be active, and to contribute to the long-lasting effect of DHE on migraine. The time course of plasma 8′-OH-DHE concentrations is plotted in FIGS. 11A and 11B. Initial summary statistics for plasma concentrations of 8′OH-DHE are provided in Table 6, below.

TABLE 6
Plasma 8′-OH-Dihydroergotamine Pharmacokinetic
Parameters (Safety Population)

| | | Cmax | Tmax | AUC₀₋ₗₐₛₜ | AUC_{0-inf} |
|---|---|---|---|---|---|
| Treatment | Statistic | (pg/mL) | (h) | (h*pg/mL) | (h*pg/mL) |
| 1.45 mg | N | 29 | 29 | 29 | 20 |
| INP104 | Mean | 55.87 | 1.417 | 414.5 | 1137 |
| | SD | 26.227 | 0.7200 | 462.64 | 624.95 |
| | CV % | 46.9 | 50.8 | 111.6 | 55.0 |
| | Median | 51.80 | 1.333 | 289.7 | 1013 |
| | Minimum | 21.3 | 0.33 | 2.78 | 162 |
| | Maximum | 151 | 4.08 | 2320 | 2710 |
| | Geometric | 50.70 | | 215.6 | 952.4 |
| | Mean | | | | |
| | Geometric | 47.5 | | 284.0 | 75.8 |
| | CV (%) | | | | |
| 1 mg | N | 31 | 31 | 31 | 25 |
| D.H.E. 45 | Mean | 387.4 | 0.148 | 500.9 | 934.3 |
| | SD | 112.57 | 0.3531 | 367.09 | 501.73 |
| | CV (%) | 29.1 | 238.8 | 73.3 | 53.7 |
| | Median | 368.0 | 0.083 | 410.7 | 891.4 |
| | Minimum | 104 | 0.07 | 144 | 288 |
| | Maximum | 592 | 2.05 | 1480 | 2280 |
| | Geometric | 368.8 | | 409.3 | 811.7 |
| | Mean | | | | |
| | Geometric | 35.3 | | 68.5 | 60.3 |
| | CV (%) | | | | |
| 2 mg | N | 10 | 10 | 10 | 4 |
| Migranal | Mean | 38.76 | 2.253 | 351.3 | 1309 |
| | SD | 15.423 | 1.2528 | 353.96 | 414.73 |
| | CV (%) | 39.8 | 55.6 | 100.8 | 31.7 |
| | Median | 36.20 | 1.925 | 202.4 | 1421 |
| | Minimum | 20.1 | 0.7 | 2.68 | 714 |
| | Maximum | 75.8 | 4.08 | 1040 | 1680 |
| | Geometric | 36.43 | | 162.4 | 1247 |
| | Mean | | | | |
| | Geometric | 37.5 | | 437.8 | 39.4 |
| | CV (%) | | | | |

| | | kₑₗ | t_{1/2} | CL/F | Vz/F |
|---|---|---|---|---|---|
| Treatment | Statistic | (1/h) | (h) | (L/h) | (L) |
| 1.45 mg | N | 20 | 20 | 20 | 20 |
| INP104 | Mean | 0.06040 | 16.856 | 1748 | 29550 |
| | SD | 0.052454 | 9.5475 | 1699.6 | 9791.6 |
| | CV % | 86.8 | 56.6 | 97.3 | 33.1 |
| | Median | 0.04376 | 15.840 | 1279 | 30910 |
| | Minimum | 0.0174 | 2.67 | 473 | 8660 |
| | Maximum | 0.259 | 39.85 | 7920 | 45400 |
| | Geometric | 0.04857 | | 1344 | 27670 |
| | Mean | | | | |
| | Geometric | 69.8 | | 75.8 | 41.6 |
| | CV (%) | | | | |
| 1 mg | N | 25 | 25 | 25 | 25 |
| D.H.E. 45 | Mean | 0.08161 | 10.533 | 1263 | 15580 |
| | SD | 0.044633 | 4.7842 | 756.51 | 4464.0 |
| | CV (%) | 54.7 | 45.4 | 59.9 | 28.7 |
| | Median | 0.07049 | 9.834 | 987.2 | 15190 |
| | Minimum | 0.0276 | 3.23 | 386 | 6800 |
| | Maximum | 0.215 | 25.16 | 3060 | 22900 |
| | Geometric | 0.07275 | | 1084 | 14900 |
| | Mean | | | | |
| | Geometric | 50.0 | | 60.3 | 32.3 |
| | CV (%) | | | | |
| 2 mg | N | 4 | 4 | 4 | 4 |
| Migranal | Mean | 0.03858 | 18.754 | 1498 | 38240 |
| | SD | 0.0096681 | 4.2336 | 650.64 | 8817.1 |
| | CV (%) | 25.1 | 22.6 | 43.4 | 23.1 |
| | Median | 0.03602 | 19.456 | 1239 | 39500 |
| | Minimum | 0.0305 | 13.39 | 1050 | 26300 |
| | Maximum | 0.0518 | 22.71 | 2460 | 47600 |
| | Geometric | 0.03773 | | 1411 | 37400 |
| | Mean | | | | |
| | Geometric CV | 24.3 | | 39.4 | 25.4 |
| | (%) | | | | |

Abbreviations:
BA = bioavailability;
CV = coefficient of variation;
SD = standard deviation.
Notes:
Pharmacokinetic parameters determined using Phoenix WinNonLin v6.3.
Pharmacokinetic parameters for Subject 100-025 after 1 mg D.H.E.45 (Period 2) were not derived due to insufficient sampling during the peak concentration phase (5- and 10-minutes samples were collected at the same clock time as the 20-minutes sample).
For 1 mg D.H.E. 45, BA was assumed to be 1.00. As such CL/F = CL and Vz/F = Vz.

These data demonstrate that intranasal administration of 1.45 mg DHE by INP104 provides equivalent systemic exposure to the active metabolite of DHE as bolus intravenous administration of 1.0 mg DHE. In addition, the metabolite was detected in only 8 subjects after Migranal® intranasal delivery, versus 24 subjects following intranasal administration of INP104.

### 6.3. Example 3: Phase III Clinical Trial

A Phase III clinical trial was conducted to test safety and therapeutic efficacy of chronic intermittent administration of intranasal Dihydroergotamine Mesylate (DHE) for 24 or 52 weeks by a delivery device (INP104) in patients with frequent migraine headaches (NCT03557333, “STOP-301”).

### 6.3.1. Study Design

The study was an interventional, open-label, single-group assignment, safety, tolerability, and exploratory efficacy study.

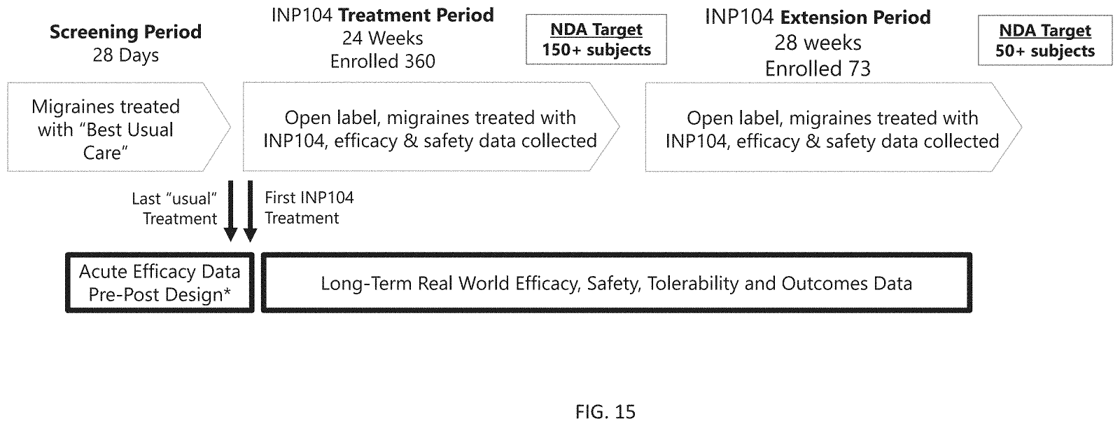

The study comprised a 4-week screening period, a 24-week treatment period for all subjects, with a 28-week extension (to 52 weeks total) for a subset of the subjects who reached Week 24, and a 2-week post-treatment follow-up period as outlined in FIG. 15. The primary objective was to access safety and tolerability of intermittent usage of INP104 via the respiratory system.

Approximately 300 to 340 patients were planned for enrollment with the goal of having at least 150 patients who had an average of 2 or more migraine and/or headaches treated with INP104 every 28 days for 24 weeks. Suitable patients continuing to the 52-week endpoint were re-consented at 24 weeks, with approximately 80 patients continuing to the 52-week treatment period in order to obtain at least 60 patients completing 52 weeks of treatment. A tabular display of study events is provided in Table 7, below:

TABLE 7
Time and event schedule

| | | | | | | | | | | | | | Follow |
|---|---|---|---|---|---|---|---|---|---|---|---|---|---|
| Period | Screening | Baseline | Treatment Period | | | | | | | | | | up |
| Week | −4 | 0 | 4 | 8 | 12 | 16 | 20 | 24/ | 26 | 36 | 42 | 52/ | 54 |
| | | | | | | | | EOT | | | | EOT | |
| Days | −28 | 0 | 28 | 56 | 84 | 112 | 140 | 168 | 182 | 252 | 294 | 364 | 378 |
| Window (days) | −7 | | ±5 | ±5 | ±5 | ±5 | ±5 | ±5 | ±10 | ±10 | ±10 | ±10 | ±14 |
| Visit | 1 | 2 | 3 | 4 | 5 | 6 | 7 | 8 | 9a | 9b | 10 | 11 | 12 |
| Informed consent | X | | | | | | | X⁸ | | | | | |
| Medical history | X | | | | | | | | | | | | |
| Confirmation of | X | X | | | | | | | | | | | |
| Eligibility | | | | | | | | | | | | | |
| Height | X | | | | | | | | | | | | |
| Weight | X | | | | | | | X | | | | X | |
| Vital signs | X | X | X | X | X | X | X | X | X | X | X | X | X |
| 12-lead ECG | X | X | | | | | | X⁹ | | | | X⁹ | |
| Physical exam | X | | | | | | | X | X | | | X | X |
| Directed exam | | X | X | X | X | X | X | | | X | X | | |
| Nasal endoscopy | X³ | | X | X | X | | | X | | X | | X | |
| UPSIT | X | X | | | X | | | X | | X | | X | |
| MIDAS and HIT-6 | X | X | | | X | | | X | | X | | X | |
| Hematology and | X | X | | | X | | | X | X | X | | X | X |
| chemistry labs | | | | | | | | | | | | | |
| Urinalysis | X | X | | | X | | | X | X | X | | X | X |
| Serology | X | | | | | | | | | | | | |
| Urine drug screen | X | | | | | | | | | | | | |
| Serum pregnancy | X | | | | | | | | | | | | |
| test | | | | | | | | | | | | | |
| Urine pregnancy | | X | X | X | X | X | X | X | X | X | X | X | X |
| test | | | | | | | | | | | | | |
| Serum FSH | X | | | | | | | | | | | | |
| Diary training | X | | | | | | | | | | | | |
| & distribution | | | | | | | | | | | | | |
| Diary check | | X | X | X | X | X | X | X | | X | X | X | |
| Training on POD | | X | | | | | | | | | | | |
| Diary Review | | X | X | X | X | X | X | X | | X | X | X | |
| Dispense IP | | X | X | X | X | X | X | X¹⁰ | | X | X | | |
| Collect unused IP | | | X | X | X | X | X | X | | X | X | X | |
| AE collection | X | X | X | X | X | X | X | X | X | X | X | X | X |
| Concomitant | X | X | X | X | X | X | X | X | X | X | X | X | X |
| (and rescue) | | | | | | | | | | | | | |
| medication | | | | | | | | | | | | | |
| Diary collection | | | | | | | | X | | | | X | |
| IP questionnaire | | | | | | | | X | | | | X | |
| (usability and | | | | | | | | | | | | | |
| effectiveness) | | | | | | | | | | | | | |

Abbreviations:
ECG = electrocardiogram;
FSH = follicle stimulating hormone;
FU = follow-up;
MIDAS = Migraine Disability Assessment;
UPSIT = University of Pennsylvania Smell Identification Test

The study was an outpatient study in frequent migraineurs (currently suffering a minimum of 2 migraines per month) but not diagnosed with chronic headache by International Classification of Headache Disorders version 3 beta (ICHD3b) criteria. During the screening period, the subjects were required to complete a migraine diary for at least 2 migraine attacks treated with the subject's usual acute treatment. If they were eligible at Visit 2, they were enrolled and provided with a supply of INP104 (up to 3 doses per week) and instructed to use IN104 when they experienced a recognizable migraine. They were instructed to use no more than 2 doses within a 24-hour period, 3 doses in a 7-day period, and 12 doses per 4-week period for their usual migraines.

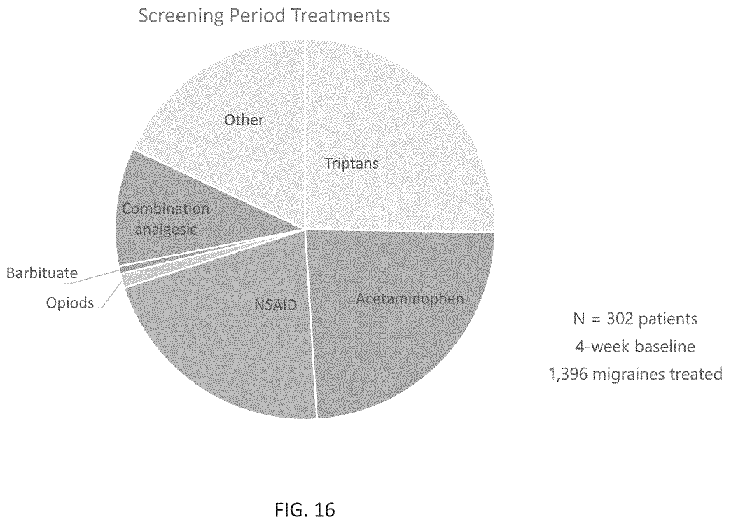

During the 28 day screening period, subjects were treated with Best Usual Care (BSC) for migraine relief as shown in FIG. 16. Typical BSC included, but was not limited to, treatment with triptans, acetaminophen, NSAID, opioids, combination analgesic, and barbiturate.

Study assessments included evaluations of clinical measures, laboratory findings, and safety reporting. Specific assessments to evaluate treatment safety included nasal endoscopy, University of Pennsylvania Smell Identification Test, and the frequency and type of adverse events. Clinical evaluations included collection of medical history, concomitant medication use, height and weight, physical examination, 12-lead electrocardiograms, and vital signs. Laboratory evaluations included hematology, serum chemistry, urinalysis, serology, and pregnancy testing for women of childbearing potential.

### 6.3.2. Key Inclusion Criteria

Subjects must have been adult males and females 18 to 65 years of age at the time of screening. Subjects must have had documented diagnosis of migraine (by International Classification of Headache Disorders version 3 beta criteria) with or without aura, with at least 2 attacks/month for previous 6 months. Subjects must have had at least 2 migraine attacks during the 28-day screening period (treated with subjects' usual treatment). Subjects who could not meet the criteria could not be rescreened. Participants were required to have been in good general health, with no significant medical history (excluding migraine), and have no clinically significant abnormalities on physical examination at screening or baseline visits.

### 6.3.3. Key Exclusion Criteria

Subjects must not have had trigeminal autonomic cephalalgias (including cluster headache, hemicrania syndromes and short-lasting, unilateral, neuralgiform headache attacks with conjunctival injection and treating), migraine aura without headache, hemiplegic migraine or migraine with brainstem aura (previously referred to as basilar migraines), chronic migraines, medication overuse headache or other chronic headache syndromes, as per by International Classification of Headache Disorders version 3 beta criteria.

Subjects must not have had a positive test for human immunodeficiency virus, hepatitis B surface antigen, or hepatitis C antibodies.

Subjects must not have had ischemic heart disease or clinical symptoms or findings consistent with coronary artery vasospasm, including Prinzmetal's variant angina. Subjects also must not have had significant risk factors for coronary artery disease or medical history of diabetes or smoking, known peripheral arterial disease, Raynaud's phenomenon, sepsis or vascular surgery (within 3 months prior to study start), or severely impaired hepatic or renal function. Subjects have a history of hypertension may be enrolled if the hypertension is stable and well-controlled on current therapies for >6 months, provided no other risk factors for coronary artery disease are present.

Subjects must not have had significant nasal congestion, physical blockage in either nostril, significantly deviated nasal septum, septal perforation, or any pre-existing nasal mucosal abnormality on endoscopy scoring 1 or more (except score 1 allowed for mucosal edema).

Subjects must not previously have shown hypersensitivity to ergot alkaloids or any of the ingredients in the drug product.

Subjects must not have previously failed to respond to intravenous DHE for treatment of migraine.

Subjects must have not have used for more than 12 days per month triptan or ergot-based medication in the 2 months prior to screening or during screening period.

### 6.3.4. Study Drug Administration

INP104 was self-administered using the 1123 POD™ Device (Impel NeuroPharma, Seattle). The dose of DHE mesylate was divided, with one spray in each nostril delivering a total target dose of 1.45 mg DHE mesylate. Subjects were instructed to use no more than 2 doses of INP104 within a 24-hour period, 3 doses in a 7-day period, and 12 doses per 4-week period.

The INP104 drug component, DHE DP, is a 3.5-mL amber glass vial filled with DHE mesylate 4 mg/mL. The formulation is identical to that in the Migranal® Nasal Spray device: a clear, colorless to faintly yellow, solution in an amber glass vial containing:

| | |
|---|---|
| dihydroergotamine mesylate, USP | 4.0 mg |
| caffeine, anhydrous, USP | 10.0 mg |
| dextrose, anhydrous, USP | 50.0 mg |
| carbon dioxide, USP | qs |
| purified water, USP | qs 1.0 mL. |

The DHE DP vial attaches to the 1123 POD Device. The 1123 POD Device has a nominal output between 175 μL/actuation pump and 205 μL/actuation pump (inclusive).

A single manual actuation of the device by the user results in the operation of the metering pump to fill the dose chamber with the DHE formulation and subsequent, but almost instantaneous, activation of the propellant canister to expel the formulation through the nozzle, as a spray, resulting in delivery to the nasal cavity of the user. The device is designed to be disposed of after successful single divided-dose drug delivery (1 spray per nostril). Actuation of the 1123 POD Device releases approximately 63 μL of HFA-134a propellant, similar to HFA exposure from metered-dose inhalers.

### 6.3.5. Outcome Measures

Subjects were evaluated for primary endpoints including evaluation of: (i) number of subjects with serious and non-serious treatment emergence adverse events; (ii) change in nasal mucosa as detected by nasal endoscopy; and (iii) change in olfactory function. The secondary endpoints included evaluation of change from baseline in rate of freedom from headache pain at 2 hours after INP104 administration; change from baseline in Most Bothersome Symptom at 2 hours after INP104 administration; change from baseline in frequency and severity of headache pain (over other time points) after INP104 administration; change from baseline in Most Bothersome Symptom at other time points after INP104 administration, frequency and severity of, and change in, nausea, phonophobia, and photophobia after INP104 administration; change in frequency and severity of migraine (measured by headache pain, nausea, phonophobia, and photophobia) by eDiary; incidence of pain relapse within 48 hours after INP104 administration; change from baseline in Migraine Disability Assessment and Headache Impact Test questionnaires; change in concomitant migraine medication use; and additional safety and tolerability are assessed by change from baseline in vital signs; change from baseline in physical examinations; change from baseline in 12-lead electrocardiogram (ECG); change from baseline in laboratory evaluations, e.g., hematology, clinical chemistry, and urinalysis. Exploratory endpoint included change from baseline in healthcare utilization for migraine.

### 6.3.6. Statistical Analysis

Statistical analysis was descriptive in nature. All subjects who received any amount of study drug (Safety Population) were included in the analyses.

Safety and tolerability were analyzed based upon the reporting of adverse events, nasal endoscopy, laboratory findings, vital sign assessments, and electrocardiogram parameters. Continuous safety data were summarized with descriptive statistics (arithmetic mean, standard deviation, median, minimum, and maximum). Categorical safety data were summarized with frequency counts and percentages. Exploratory efficacy analyses focused on summarizing the following over time: frequency and severity of migraine symptoms captures in the migraine diary; Migraine Disability Assessment and Headache Impact Test questionnaire scores; and use of concomitant migraine medications.

### 6.3.7. Results

### Summary of Headaches

The 354 patients included in the 24-week full safety set experienced 4605 headaches that were treated with INP104, of which 4515 were considered migraine attacks (Table 8). In the 24 week primary safety set, 185 patients experienced 3582 headaches that were treated with INP104, of which 3530 were considered migraine attacks.

At each postbaseline 4-week interval, >80% and ≥85% of reported headaches were migraine attacks in the 24 week full and primary safety sets, respectively. The INP104 treatment for migraine attacks at each 4-week interval primarily occurred <2 hours after the migraine attack began (>70% of migraines). In the 24-week full and primary safety sets, the number of reported headaches overall and the number of migraine attacks experienced during each postbaseline 4-week interval decreased substantially, especially in the first 12 weeks, compared with the baseline measure of the total headaches treated with standard-of-care acute medication.

In the 52-week full safety set, 73 patients experienced 2642 headaches that were treated with INP104, of which 2617 were considered migraine attacks. In the 52-week primary safety set, 55 patients experienced 2205 headaches that were treated with INP104, of which 2189 were considered migraine attacks.

TABLE 8
Summary of Headaches by 4-Week Interval -
24-Week Treatment Period

| | 24-Week Treatment Period | | | |
|---|---|---|---|---|
| | Full | | Primary | |
| | N = 354 | | N = 185 | |
| 4-Week Interval | Actual Value | | Actual Value | |
| Total number of IP-treated headaches | 4605 | | 3582 | |
| Total number of IP-treated migraines | 4515 | | 3530 | |
| Baseline | | | | |
| n | 354 | | 183 | |
| Number of headaches | 1999 | | 1086 | |
| Migraine, n (%)^{a} | 1654 | (82.7) | 923 | (84.2) |
| Nonmigraine, n (%)^{a} | 345 | (17.3) | 173 | (15.8) |
| Weeks 1-4 | | | | |
| n^{b} | 354 | | 185 | |
| Number of headaches | 1570 | | 945 | |
| Migraine, n (%)^{b} | 1303 | (83.0) | 801 | (84.8) |
| Nonmigraine, n (%)^{b} | 267 | (17.0) | 144 | (15.2) |
| Number of IP-treated headaches, n (%)^{b} | 1123 | (71.5) | 723 | (76.5) |
| Migraine, n (%)^{b} | 1099 | (70.0) | 711 | (75.2) |
| Nonmigraine, n (%)^{b} | 24 | (1.5) | 12 | (1.3) |
| Weeks 5-8 | | | | |
| n^{b} | 331 | | 185 | |
| Number of headaches | 1155 | | 794 | |
| Migraine, n (%)^{b} | 980 | (84.8) | 699 | (88.0) |
| Nonmigraine, n (%)^{b} | 175 | (15.2) | 95 | (12.0) |
| Number of IP-treated headaches, n (%)^{b} | 875 | (75.8) | 636 | (80.1) |
| Migraine, n (%)^{b} | 855 | (74.0) | 631 | (79.5) |
| Nonmigraine, n (%)^{b} | 20 | (1.7) | 5 | (0.6) |
| Weeks 9-12 | | | | |
| n^{b} | 302 | | 185 | |
| Number of headaches | 929 | | 705 | |
| Migraine, n (%)^{b} | 785 | (84.5) | 620 | (87.9) |
| Nonmigraine, n (%)^{b} | 144 | (15.5) | 85 | (12.1) |
| Number of IP-treated headaches, n (%)^{b} | 724 | (77.9) | 581 | (82.4) |
| Migraine, n (%)^{b} | 708 | (76.2) | 570 | (80.9) |
| Nonmigraine, n (%)^{b} | 16 | (1.7) | 11 | (1.6) |
| Weeks 13-16 | | | | |
| n^{b} | 286 | | 184 | |
| Number of headaches | 824 | | 673 | |
| Migraine, n (%)^{b} | 726 | (88.1) | 608 | (90.3) |
| Nonmigraine, n (%)^{b} | 98 | (11.9) | 65 | (9.7) |
| Number of IP-treated headaches, n (%)^{b} | 667 | (80.9) | 567 | (84.2) |
| Migraine, n (%)^{b} | 653 | (79.2) | 556 | (82.6) |
| Nonmigraine, n (%)^{b} | 14 | (1.7) | 11 | (1.6) |
| Weeks 17-20 | | | | |
| n^{b} | 275 | | 185 | |
| Number of headaches | 777 | | 636 | |
| Migraine, n (%)^{b} | 676 | (87.0) | 577 | (90.7) |
| Nonmigraine, n (%)^{b} | 101 | (13.0) | 59 | (9.3) |
| Number of IP-treated headaches, n (%)^{b} | 624 | (80.3) | 540 | (84.9) |
| Migraine, n (%)^{b} | 618 | (79.5) | 536 | (84.3) |
| Nonmigraine, n (%)^{b} | 6 | (0.8) | 4 | (0.6) |
| Weeks 21-24 | | | | |
| n^{b} | 265 | | 185 | |
| Number of headaches | 711 | | 617 | |
| Migraine, n (%)^{b} | 638 | (89.7) | 569 | (92.2) |
| Nonmigraine, n (%)^{b} | 73 | (10.3) | 48 | (7.8) |
| Number of IP-treated headaches, n (%)^{b} | 592 | (83.3) | 535 | (86.7) |
| Migraine, n (%)^{b} | 582 | (81.9) | 526 | (85.3) |
| Nonmigraine, n (%)^{b} | 10 | (1.4) | 9 | (1.5) |

IP = investigational product (INP104)
Note:
Baseline was defined for each patient by averaging the data recorded within 28 days prior to patient's enrollment to the study on Day 0. If no measurement of a parameter was collected before the patient's enrollment to the study on Day 0, then the baseline was set to missing.
^{a}Percentages were based on the total number of headaches during the 4-week interval.
^{b}Number of patients who had any nonmissing data in the electronic diary during the 4-week interval.

Migraine Attacks Pain-Free at 2 Hours after Administration of INP104

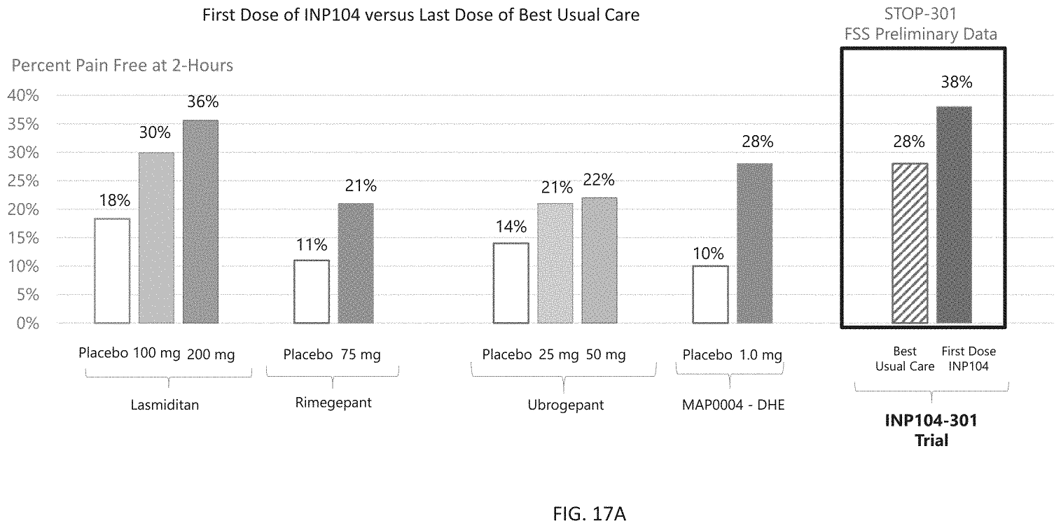

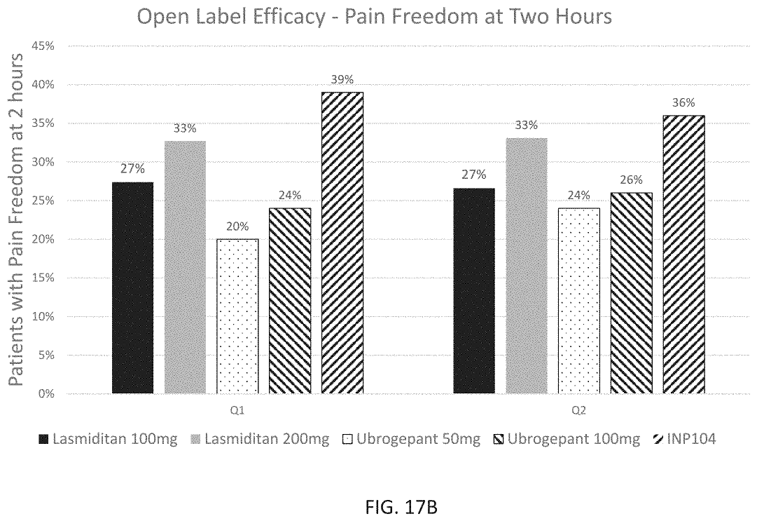

The percentage of clinical trial patients free of pain at 2 hours following the last dose of Best Usual Care and following the first dose of INP104 are plotted in FIG. 17A (“STOP-301 FSS Preliminary Data”) and compared to results reported in the literature for lasmiditan, rimegepant, ubrogepant, and MAP0004-DHE. Subjects were on Best Usual Care treatments including triptans, acetaminophen, NSAID, barbiturate, opioids, combination analgesics and others, as shown in FIG. 16. The results show that treatment with INP104 provides unsurpassed benefit with 38% of subjects having pain freedom at 2 hours, which is better than each patient's last dose of Best Usual Care, and better than prior reports from other treatments reported in the literature—i.e., administration of DHE by oral inhalation (MAP0004-DHE), or administration of lasmiditan, rimegepant, or ubrogepant. The benefit of pain freedom and the better therapeutic outcome of INP104 as compared to lasmiditan and ubrogepant was sustained over the long term. FIG. 17B.

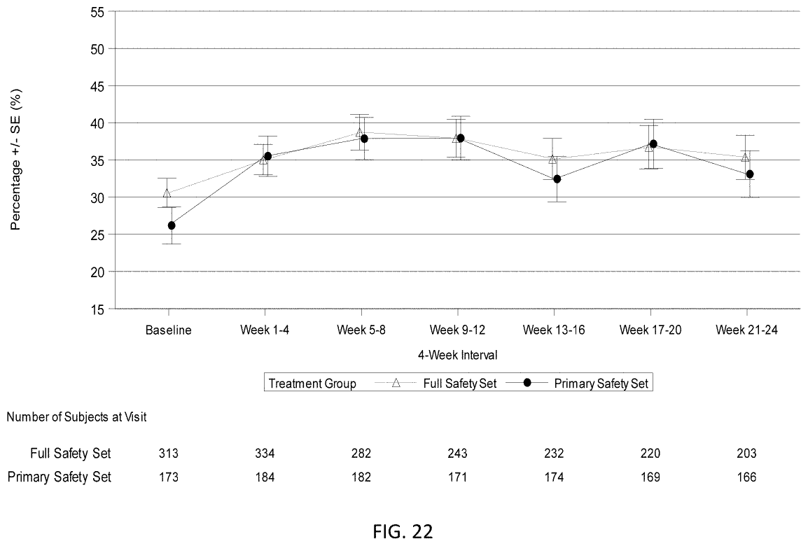

In the 24-week treatment period, the mean percentage of migraine attacks that were pain free 2 hours after INP104 administration was higher at each postbaseline 4-week interval compared with the equivalent baseline measure following Best Usual Care acute medication treatment (baseline means: 30.59% and 26.19% in the 24-week full and primary safety sets, respectively; FIG. 22). The mean increases from the baseline standard-of-care pain-free at 2 hours measure ranged from 2.60% to 10.25% in the 24 week full safety set and 4.96% to 11.55% in the 24-week primary safety set.

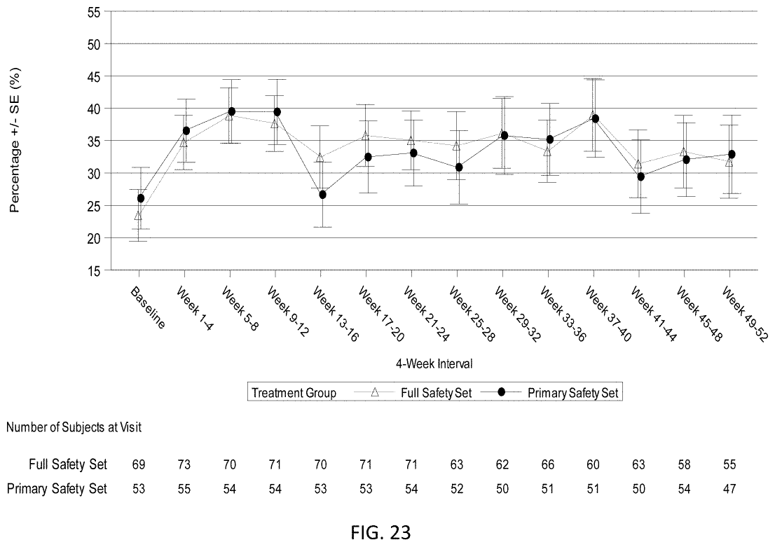

The results for the 52-week treatment period (full and primary safety sets) were consistent with the results for the 24-week treatment period (full and primary safety sets; FIG. 23).

### Most Bothersome Symptom (MBS)

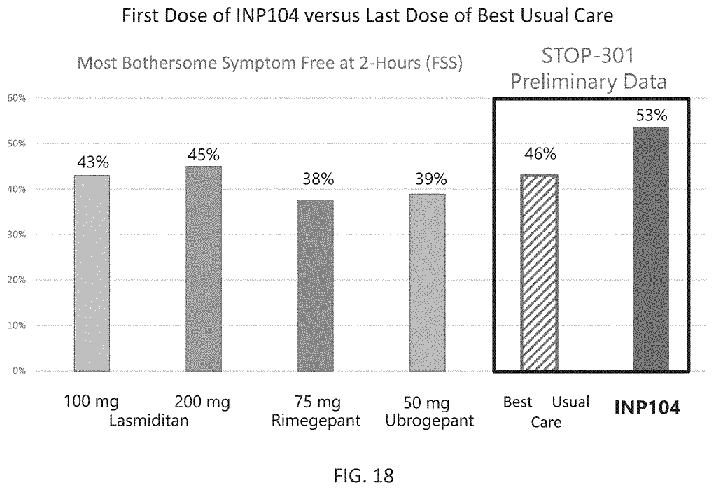

Changes from baseline in the most bothersome symptom were also measured. Percentages of patients free of the most bothersome symptom at 2 hours after first dose of INP104 administration or last dose of Best Usual Care are plotted in FIG. 18 (“STOP-301 Preliminary Data”) and compared to results reported in the literature for lasmiditan, rimegepant, and ubrogepant. INP104 first dose demonstrates unsurpassed benefit with 53% of subjects having most bothersome symptom relief at 2 hours, whereas lower percentages of subjects had relief when they received the last dose of Best Usual Care. These data are compared to data reported in the literature for lasmiditan and ubrogepant.

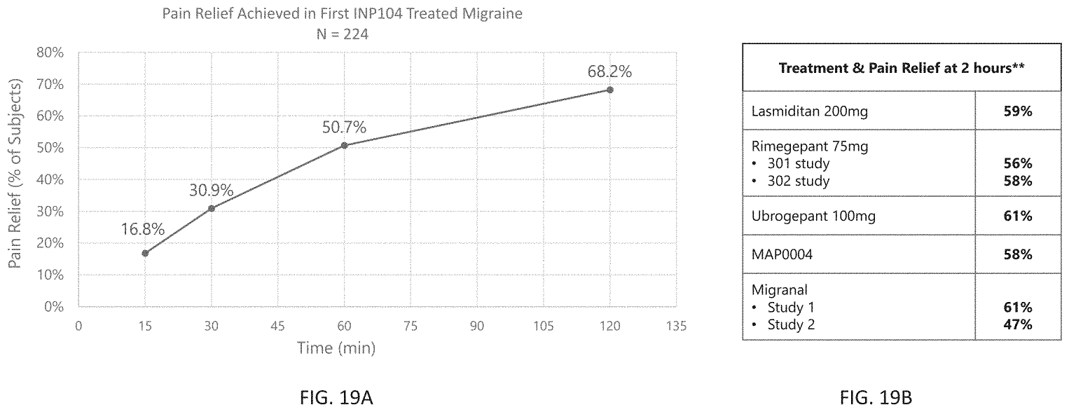

Percentages of patients having pain relief (% subjects) after treatment with a first dose of INP104 are plotted over time, from 15 min to 120 min after the intranasal administration of DHE, in FIG. 19A. In the analysis, subjects having an improvement from moderate or severe pain to mild or no pain, or from mild pain to no pain, were counted to be the subjects having a pain relief. Subjects having pain relief gradually increased over time from 16.8% at 15 min to 68.2% at 2 hours after they received the INP104 first dose. FIG. 19B tabulates reports in the literature for lasmiditan, rimegepant, ubrogepant, MAP0004, and Migranal®.

Throughout the 120 minute period, these therapeutic effects of INP104 were better than results reports for other migraine treatment methods, as provided in FIGS. 19A and B. For example, 16.8% of subjects had pain relief at 15 min after treatment with INP104, whereas 9% of subjects and 8% of subjects had pain relief at 15 min after treatment with MAP0004 and Rimegepant, respectively. At 120 min after treatment with INP104, 68.2% of subjects had pain relief, whereas 47%-61% of subjects had pain relief at 120 min after treatment with Lasmiditan (200 mg), Rimegepant (75%), Ubrogepant (100 mg), MAP0004, or Migranal. FIG. 19B. The pain relieving effects of INP104 were consistent and reproducible. About 30% of patients treated achieved mild to no pain at 2 hours in all migraines treated with INP104, and about 60% of patients achieved mild to no pain at 2 hours in 75% of migraines treated with INP104.

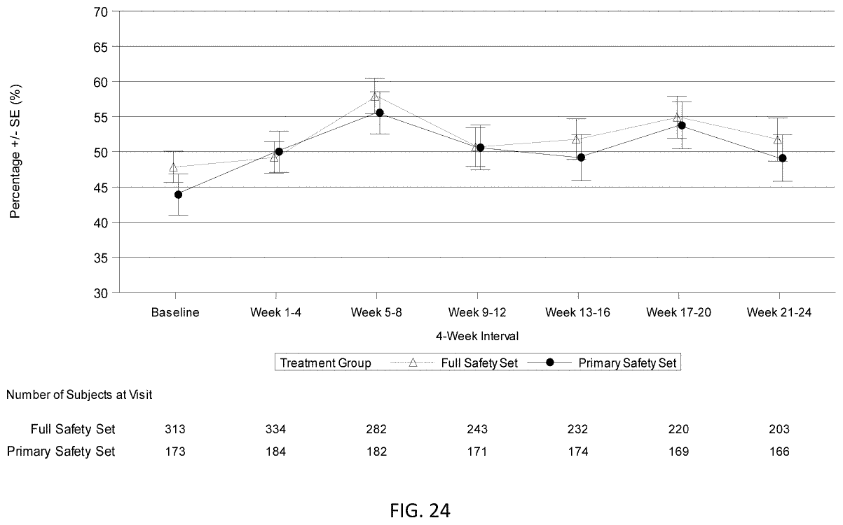

In the 24-week treatment period, the mean percentage of migraine attacks that were MBS free 2 hours after INP104 administration was higher at each postbaseline 4-week interval compared with the equivalent baseline measure following Best Usual Care acute medication treatment (baseline means: 47.85% and 43.91% in the 24-week full and primary safety sets, respectively; FIG. 24). The mean increases from the baseline measure (i.e., patients' standard treatment) for patients with data at baseline and a subsequent time point ranged from 3.07% to 9.71% in the 24 week full safety set and from 4.42% to 11.10% in the 24 week primary safety set, with the exception of a single minimal mean decrease from baseline in the 24-week full safety set at the Week 1-4 interval (−0.63%).

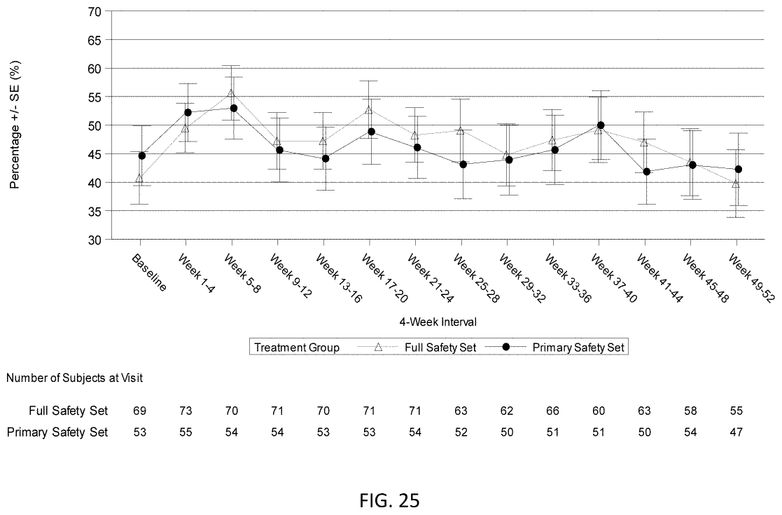

The results for the 52-week treatment period (full and primary safety set) were consistent with the results for the 24-week treatment period (full and primary safety sets; FIG. 25).

### Incidence of Pain Relapse

Pain Relapse 24 Hours after Treatment with INP104:

Percentage of migraine attacks with pain relapse by 24 hours and by 48 hours after INP104 administration was measured, where pain relapse was defined as a migraine that was pain free 2 hours after INP104 administration and then pain was present before or at 24 and/or 48 hours after INP104 administration; or there was an onset of a new headache before the 24- or 48-hour time point.

The patients included at baseline and each postbaseline time point were those with at least 1 migraine that was pain free 2 hours after non-INP104 (baseline) or INP104 (postbaseline) administration at each specific 4 week interval.

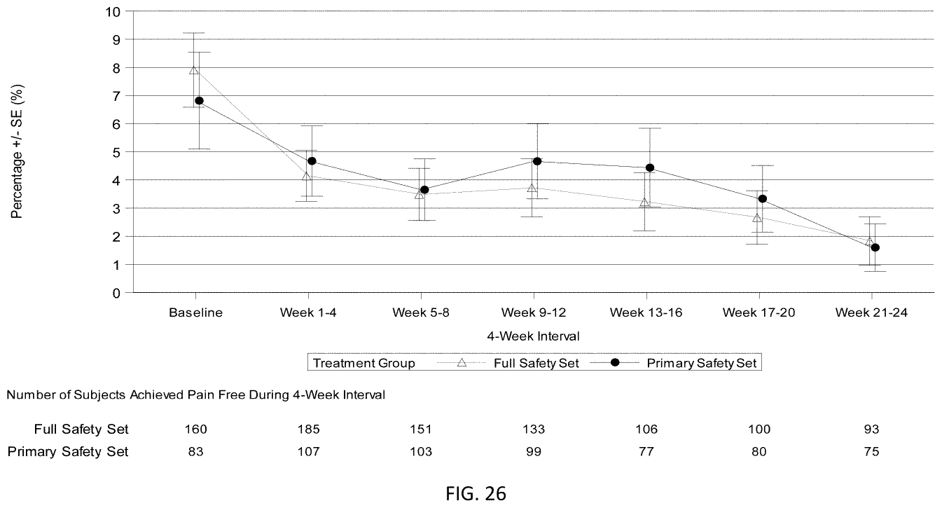

In the 24-week treatment period, the mean percentage of migraine attacks with pain relapse 24 hours after IP administration was lower at each postbaseline 4-week interval compared with the equivalent baseline measure following Best Usual Care acute medication treatment (FIG. 26).

The results for the 52-week treatment period (full and primary safety set) were consistent with the results for the 24-week treatment period.

Pain Relapse 48 Hours after Treatment with INP104:

In the 24 week full and primary safety sets, the mean percentage of migraines with pain relapse 48 hours after treatment with INP104 was lower at each postbaseline 4-week interval compared with the equivalent baseline measure following Best Usual Care acute medication treatment (Table 9).

TABLE 9
Summary of Percentage of Migraine Attacks with Pain Relapse at 48 Hours
After Treatment with INP104 by 4-Week Interval - 24-Week Treatment Period

| | 24-Week Safety Set | | | | | | | |
|---|---|---|---|---|---|---|---|---|
| | Full | | | | Primary | | | |
| | N = 354 | | | | N = 185 | | | |
| 4-Week Interval | | | Change from | | | | Change from | |
| Percentage^{a} | Actual Value | | Baseline^{b} | | Actual Value | | Baseline^{b} | |
| Baseline | | | | | | | | |
| n^{c} | 148 | | | | 77 | | | |
| Mean (SD) | 15.85 | (22.892) | | | 12.26 | (21.461) | | |
| Week 1-4 | | | | | | | | |
| n^{c} | 176 | | 93 | | 102 | | 53 | |
| Mean (SD) | 8.36 | (17.262) | −9.06 | (27.085) | 9.81 | (18.368) | −5.97 | (24.733) |
| Weeks 5-8 | | | | | | | | |
| n^{c} | 148 | | 83 | | 102 | | 56 | |
| Mean (SD) | 8.50 | (17.675) | −7.47 | (29.402) | 10.17 | (18.789) | −2.27 | (28.412) |
| Weeks 9-12 | | | | | | | | |
| n^{c} | 129 | | 68 | | 95 | | 54 | |
| Mean (SD) | 6.45 | (15.025) | −8.43 | (27.059) | 7.32 | (15.996) | −6.64 | (27.329) |
| Weeks 13-16 | | | | | | | | |
| n^{c} | 103 | | 51 | | 75 | | 39 | |
| Mean (SD) | 8.93 | (18.696) | −5.68 | (33.311) | 11.60 | (20.560) | −0.30 | (33.641) |
| 95% CI^{d} | (5.278, 12.586) | | (−15.046, 3.692) | | (6.870, 16.330) | | (−11.204, 10.606) | |
| Weeks 17-20 | | | | | | | | |
| n^{c} | 99 | | 54 | | 79 | | 46 | |
| Mean (SD) | 6.73 | (15.355) | −7.33 | (28.327) | 8.43 | (16.783) | −6.95 | (29.912) |
| Weeks 21-24 | | | | | | | | |
| n^{c} | 92 | | 52 | | 74 | | 45 | |
| Mean (SD) | 5.08 | (13.825) | −5.43 | (27.599) | 5.64 | (14.293) | −4.43 | (28.560) |

CI = confidence interval; IP = investigational product; SD = standard deviation.
Note:
Migraine pain relapse at 48 hours was defined as a migraine that was pain free at 2 hours after migraine medication administration and then was not pain free at 24 or 48 hours after medication or there was an onset of a new headache prior to 48 hours after IP administration.
^{a}For each patient, percentage at baseline was the average of all non-IP-treated migraine within 28 days prior to patient's enrollment to the study on Day 0. Percentage at each postbaseline 4-week interval was the average of all IP-treated migraines during the same interval.
^{b}For change from baseline summaries, only patients with nonmissing values at both baseline and postbaseline were included.
^{c}n is the number of patients who achieved freedom from pain 2 hours after medication and had data at the specific 4-week interval.
^{d}Wald 95% CI.

### Migraine Attack Frequency

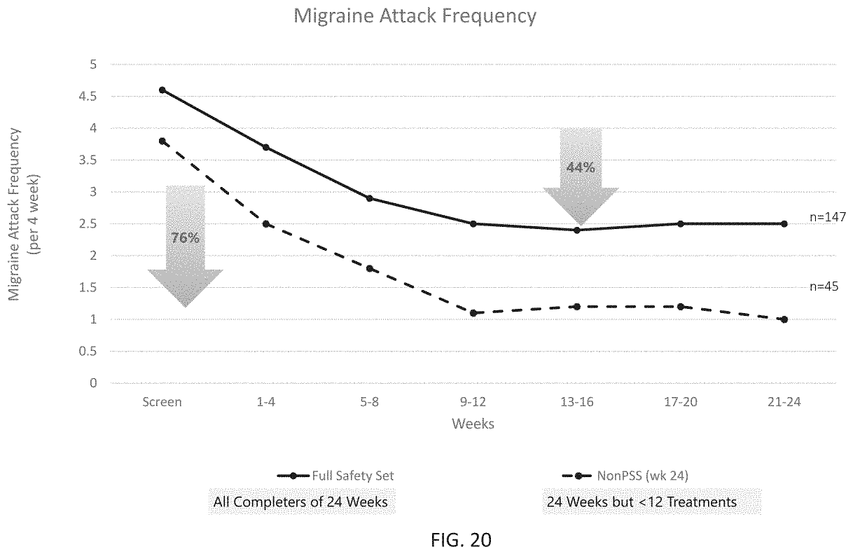

Furthermore, as shown in FIG. 20, INP104 administration on a repeat dose schedule demonstrated preventive effects, significantly reducing migraine frequency over time. The reduction of migraine frequency was sustained throughout the 6-month (or 24-week) treatment period. The 147 patients who completed the 24-week treatment period showed about 44% reduction in the migraine frequency, as shown in FIG. 20. These patients had about 4.5 migraine attacks per month on average during the screening period but only about 2.5 migraine attacks per month after treatment with INP104. A subgroup of 45 patients who completed the 24-week treatment period but who had fewer than 12 migraines during the 24-week treatment period—and thus had fewer than 12 INP104 treatments over the treatment period—showed about 76% reduction in migraine frequency. This subgroup of patients had about 3.7 migraine attacks per month on average during the screening period, but they had only about 1 migraine attack per month after treatment with INP104 for about 9-12 weeks.

### Changes in MIDAS Scores

The MIDAS category grades (I, II, III, IVa, and IVb) associated with score ranges are provided in Table 10. A higher score indicates greater headache-related disability.

TABLE 10
MIDAS Category Range

| Score Range | Grade | Definition |
|---|---|---|
| 0-5 | I | Minimal or infrequent disability |
| 6-10 | II | Mild or infrequent disability |
| 11-20 | III | Moderate disability |
| 21-40 | IVa | Severe disability - subgroup a |
| 41-270 | IVb | Severe disability - subgroup b |

At baseline, 102 of 354 patients (28.8%) and 54 of 185 patients (29.2%) in the 24 week full and primary safety sets, respectively, were Grade III (moderate disability) and 98 (27.7%) and 55 patients (29.7%), respectively, were Grade IVa (Table 14.2.2.1a), based on the percentage of patients at each severity grade. The mean MIDAS total score in each 24 week safety set decreased from the baseline at each visit (Table 11), with mean changes from baseline ranging from −5.5 to −7.4 in the 24-week full safety set and from −5.3 to −7.3 in the 24-week primary safety set. The mean scores at each of the 3 postbaseline visits were at or close to Grade III in each 24-week safety set. Patients were most frequently categorized to Grade III at each postbaseline visit in the 24-week full safety set (range: 31.0% to 34.4%) and the 24 week primary safety set (range: 33.5% to 37.2%).

The results for the 52-week treatment period (full and primary safety sets) were consistent with the results for the 24-week treatment period (full and primary safety sets).

TABLE 11
Summary of MIDAS Total Scores by Visit - 24-Week Treatment Period

| | 24-Week Safety Set | | | |
|---|---|---|---|---|
| | Full | | Primary | |
| | N = 354 | | N = 185 | |
| | | Change from | | Change from |
| Visit | Actual Value | Baseline^{a} | Actual Value | Baseline^{a} |
| Baseline | | | | |
| n | 354 | | 185 | |
| Mean (SD) | 25.1 (22.30) | | 25.4 (20.89) | |
| Median | 19.0 | | 19.0 | |
| Min, Max | 0, 180 | | 0, 122 | |
| 95% CI^{b} | (22.76, 27.42) | | (22.36, 28.42) | |
| Week 12 | | | | |
| n^{c} | 283 | 283 | 181 | 181 |
| Mean (SD) | 18.4 (17.08) | −5.5 (18.68) | 20.1 (18.03) | −5.3 (19.16) |
| Median | 14.0 | −3.0 | 15.0 | −3.0 |
| Min, Max | 0, 105 | −78, 72 | 0, 105 | −66, 72 |
| 95% CI^{b} | (16.44, 20.44) | (−7.69, −3.32) | (17.47, 22.76) | (−8.15, −2.53) |
| Week 24 | | | | |
| n^{c} | 209 | 209 | 145 | 145 |
| Mean (SD) | 17.4 (16.52) | −7.4 (17.56) | 18.9 (16.90) | −7.3 (17.46) |
| Median | 14.0 | −4.0 | 15.0 | −4.0 |
| Min, max | 0, 105 | −61, 50 | 0, 105 | −61, 50 |
| 95% CI^{b} | (15.17, 19.68) | (−9.75, −4.96) | (16.11, 21.66) | (−10.20, −4.47) |
| End of treatment | | | | |
| n^{c} | 332 | 332 | 185 | 185 |
| Mean (SD) | 18.0 (16.41) | −6.1 (17.10) | 19.1 (16.99) | −6.3 (17.18) |
| Median | 14.0 | −4.0 | 15.0 | −3.0 |
| Min, Max | 0, 105 | −74, 50 | 0, 105 | −62, 50 |
| 95% CI^{b} | (16.24, 19.78) | (−7.99, −4.30) | (16.61, 21.54) | (−8.81, −3.82) |

CI = confidence interval; Max = maximum; MIDAS = Migraine Disability Assessment; Min = minimum; SD = standard deviation.
Note:
The MIDAS total score was categorized into the following grades: I (0-5) little to no disability; II (6-10) mild disability; III (11-20) moderate disability; IVa (21-40) severe disability - subgroup a; and IVb (41-270) severe disability - subgroup b.
Baseline was defined as the last nonmissing assessment before the date of patient's enrollment to the study on Day 0. The end-of-treatment visit was the last nonmissing assessment in the 24-week treatment period. For categorical summaries, percentages were based on the n-value at each visit.
^{a}For change from baseline summaries, only patients with nonmissing values at both baseline and postbaseline were included.
^{b}Wald 95% CI.
^{c}Postbaseline only included data from patients who started the first investigational product on/before the visit evaluated.

### Changes in HIT-6 Scores

Life impact categories (little to none, some, substantial, and severe) associated with score ranges are provided in Table 12. A higher score indicates greater headache related disability.

TABLE 12
HIT-6 Category Range

| Score Range | Life Impact |
|---|---|
| ≥60 | Severe |
| 56-59 | Substantial |
| 50-55 | Some |
| ≤49 | Little to none |

For the 24-week full and primary safety sets, the baseline mean HIT-6 total scores were 63.8 and 64.2, respectively (Table 13), and the majority (>80%) of patients had a baseline score indicating a severe life impact (296 of 354 patients [83.6%] and 158 of 185 patients [85.4%], respectively). At the Week 12, Week 24, and end-of-treatment visits, the mean HIT-6 total score showed small (<1) decreases from the baseline at each visit, and headache-related disability for most patients (>70%) remained severe.

The results for the 52-week treatment period (full and primary safety sets) were consistent with the results for the 24-week treatment period (full and primary safety sets).

TABLE 13
Summary of HIT-6 Total Scores by Visit - 24-Week Treatment Period

| | 24-Week Safety Set | | | |
|---|---|---|---|---|
| | Full | | Primary | |
| | N = 354 | | N = 185 | |
| | | Change from | | Change from |
| Visit | Actual Value | Baseline^{a} | Actual Value | Baseline^{a} |
| Baseline | | | | |
| n | 354 | | 185 | |
| Mean (SD) | 63.8 (5.37) | | 64.2 (5.39) | |
| Median | 64.0 | | 64.0 | |
| Min, Max | 43, 78 | | 48, 78 | |
| 95% CI^{b} | (63.29, 64.41) | | (63.40, 64.96) | |
| Week 12 | | | | |
| n^{c} | 283 | 283 | 181 | 181 |
| Mean (SD) | 62.7 (5.89) | −0.9 (4.91) | 63.6 (5.62) | −0.6 (5.01) |
| Median | 63.0 | 0.0 | 64.0 | 0.0 |
| Min, max | 47, 76 | −16, 22 | 48, 76 | −16, 22 |
| 95% CI^{c} | (61.99, 63.37) | (−1.46, −0.31) | (62.75, 64.40) | (−1.29, 0.18) |
| Week 24 | | | | |
| n^{c} | 209 | 209 | 145 | 145 |
| Mean (SD) | 63.0 (6.85) | −0.7 (5.64) | 64.0 (6.63) | −0.2 (5.63) |
| Median | 63.0 | 0.0 | 65.0 | 0.0 |
| Min, Max | 42, 78 | −26, 20 | 42, 78 | −26, 20 |
| 95% CI^{b} | (62.10, 63.97) | (−1.49, 0.05) | (62.92, 65.09) | (−1.10, 0.74) |
| End of treatment | | | | |
| n^{c} | 331 | 331 | 185 | 185 |
| Mean (SD) | 62.9 (6.56) | −0.8 (5.28) | 63.8 (6.31) | −0.4 (5.55) |
| Median | 63.0 | 0.0 | 64.0 | 0.0 |
| Min, Max | 36, 78 | −26, 20 | 42, 78 | −26, 20 |
| 95% CI^{b} | (62.21, 63.63) | (−1.38, −0.24) | (62.90, 64.73) | (−1.17, 0.44) |

CI = confidence interval; HIT-6 = Headache Impact Test-6; Max = maximum; Min = minimum SD = standard deviation.
Note:
The HIT-6 score was categorized into the following grades: little to none (≤49); some (50-55); substantial (56-59); and severe (≥60).
Baseline was defined as the last nonmissing assessment before the date of patient's enrollment to the study on Day 0. The end-of-treatment visit was the last nonmissing assessment in the 24-week treatment period. For categorical summaries, percentages were based on the n-value at each visit.
^{a}For change from baseline summaries, only patients with nonmissing values at both baseline and postbaseline were included.
^{b}Wald 95% CI.
^{c}Postbaseline only included data from patients who started the first investigational product on/before the visit evaluated.

### Efficacy Conclusions

In the 24-week full safety set, 354 patients experienced 4605 total headaches that were treated with INP 104, of which 4515 were migraine attacks, while in the 24-week primary safety set, 185 patients experienced 3582 total headaches that were treated with INP104, of which 3530 were migraine attacks.

In the 52-week full safety set, 73 patients experienced 2642 headaches that were treated with INP 104, of which 2617 were considered migraine attacks, while in the 52-week primary safety set, 55 patients experienced 2205 headaches that were treated with INP104, of which 2189 were considered migraine attacks.

The number of migraine attacks experienced during each postbaseline 4-week interval in the 24-week treatment period decreased compared with the baseline measure of the total headaches treated with Best Usual Care acute medication treatment, with a substantial reduction in the first 12 weeks of INP104 treatment.

The mean percentage of migraine attacks that were pain free 2 hours after INP104 administration in the 24-week treatment period was higher at each postbaseline 4-week interval compared with the equivalent baseline measure following standard-of-care acute medication treatment, with a generally consistent postbaseline effect seen over time.

The mean percentage of migraine attacks that were pain free 2 hours after INP104 administration in the 24-week treatment period was higher at each postbaseline 4-week interval compared with the equivalent baseline measure following Best Usual Care acute medication treatment, with a generally consistent postbaseline effect seen over time.

The mean MIDAS total score in each 24-week safety set decreased from baseline at each visit, with mean scores at each postbaseline visit that were at or close to Grade III (moderate disability).

Overall, the efficacy results for the 52-week treatment period (full and primary safety sets) were consistent with the results for the 24-week treatment period (full and primary safety sets).

### Other Evaluations

### Patient's Impression of INP104 Usability/Effectiveness:

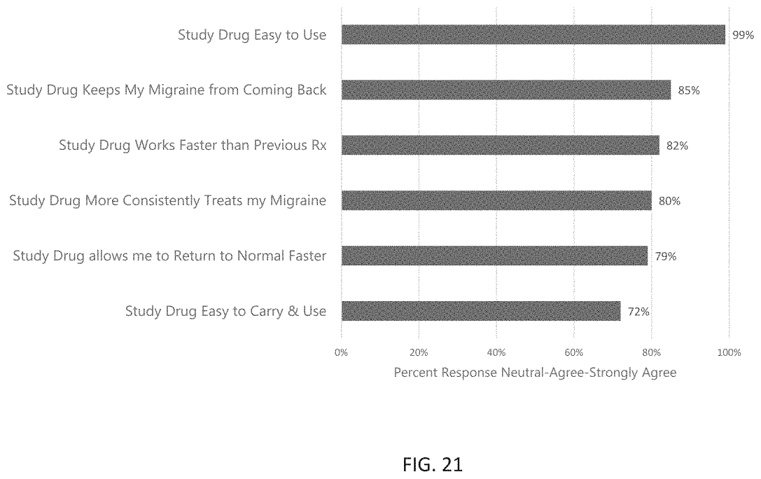

The majority of patients (>90%) in the 24-week full and primary safety sets completed the questionnaire. In the 24-week full and primary safety sets, most patients strongly agreed (50.0% and 53.8%, respectively) or agreed (33.6% and 35.3%, respectively) that the INP104 was easy to use. As shown in FIG. 21, INP104 met the needs of patients in addressing their migraine. High percentages of patients treated with INP 104 were neutral, agreed or strongly agreed that INP104 is easy to use, keeps migraine from coming back, works faster than previous drugs, consistently treats migraine, allows him or her to return to normal faster, and is easy to carry and use.

For the questions comparing INP104 with previous patient use of prescription medications, patient responses were mixed across the neutral, agree, and strongly agree categories for each question, ranging between 20% and 30% for comparison of whether the product worked faster, kept migraines from coming back for a longer time, allowed a return to normal activities faster, or more consistently relieved each migraine. Patients included in the 24 week full and primary safety sets strongly agreed (27.4% and 33.5%, respectively) or agreed (31.4% and 34.7%, respectively) that, if INP104 were commercially available, they would request a prescription of the INP104 from their physician. Compared with other nasal migraine medications, discomfort related to the INP104 device and bad taste related to the INP104 was <20% in any single agree category (i.e., strongly agree or agree), likely because >35% of patients previously had not used a nasal migraine medication.

### Healthcare Utilization:

Baseline healthcare utilization related to migraine attacks within the prior 12 months was collected in the electronic case report form (eCRF) at screening, and determined after baseline based on eDiary entries. Baseline data collection was per patients' recall and not a review of patient records. Postbaseline results were based on utilization in context of headaches or migraine attacks. For example, patients recorded in the eDiary any visit to a clinic/physician office that was unplanned and due to a headache or migraine attack; however, such visits were not recorded at the study site as unscheduled study visits in the eCRF. Baseline and postbaseline healthcare utilization and associated exposure-adjusted event rate (EAER values) are summarized in Table 14, where percentages are based on the total number of events at each relevant time point. Note that new or changed prescriptions for acute migraine/preventive migraines and preventive procedures for migraine were collected at baseline only.

A total of 162 baseline healthcare utilization events were reported for the 24-week full safety set, with the highest baseline occurrence (>20% of total events) for new or changed prescriptions for preventive migraines (41 events [25.3%]) and emergency room visits (35 events [21.6%]; Table 14). Postbaseline, 33 events were reported, with the only reported events being emergency room visits (5 events [15.2%]) and unplanned clinic/physician office visits (28 events [84.8%]); however, a serious TEAE of status migrainosus required an emergency room visit and hospitalization but, per eDiary entry, was only recorded as an emergency room visit. The EAER for emergency room visits was lower after baseline than at baseline (2.6 vs 9.9) but was higher for unplanned clinic/physician office visits (14.6 vs 8.8). Similar results were reported for the 24 week primary safety set.

TABLE 14
Summary of Healthcare Utilization

| | 24-Week Safety Set | | | | | |
|---|---|---|---|---|---|---|
| | Full | | | Primary | | |
| | N = 354 | | | N = 185 | | |
| Category | n (%) | | EAER | n (%) | | EAER |
| Baseline^{a} | | | | | | |
| Total healthcare utilization | 162 | | | 84 | | |
| events | | | | | | |
| Hospitalization | 2 | (1.2) | 0.6 | 1 | (1.2) | 0.5 |
| Emergency room visit | 35 | (21.6) | 9.9 | 17 | (20.2) | 9.2 |
| Urgent care visit | 25 | (15.4) | 7.1 | 15 | (17.9) | 8.1 |
| Unplanned clinic/physician | 31 | (19.1) | 8.8 | 11 | (13.1) | 5.9 |
| office visit | | | | | | |
| New or changed prescription | 22 | (13.6) | 6.2 | 14 | (16.7) | 7.6 |
| for acute migraine | | | | | | |
| New or changed prescription | 41 | (25.3) | 11.6 | 21 | (25.0) | 11.4 |
| for preventive migraine | | | | | | |
| New or changed preventive | 6 | (3.7) | 1.7 | 5 | (6.0) | 2.7 |
| procedure for migraine | | | | | | |
| Postbaseline^{b} | | | | | | |
| Total healthcare utilization | 33 | | | 27 | | |
| events | | | | | | |
| Hospitalization | 0 | | 0 | 0 | | 0 |
| Emergency room visit | 5 | (15.2) | 2.6 | 3 | (11.1) | 2.3 |
| Urgent care visit | 0 | | 0 | 0 | | 0 |
| Unplanned clinic/physician | 28 | (84.8) | 14.6 | 24 | (88.9) | 18.4 |
| office visit | | | | | | |

EAER = exposure-adjusted event rate.
Note:
The EAER was the expected number of specific events per 100-person years of exposure. It was defined as 100 times the number of events divided by the total exposure time (in years) among patients included in each identified analysis set. Patients with multiple occurrences of each specific event were counted multiple times. The total exposure time in years was calculated by dividing the sum of exposure time in days across all patients included in each identified analysis set by 365.25.
Percentages were based on the total number of events at relevant time points unless otherwise specified.
^{a}Baseline was defined as healthcare utilization data over the previous 12 months at the time of screening. Baseline exposure time for each patient was 12 months.
^{b}Postbaseline was defined as healthcare utilization data collected from a patient's enrollment to the study on Day 0 to the end of the study. The postbaseline exposure time for a patient was the patient's time in the study.

### Safety

INP104 was demonstrated to be safe and well tolerated by the subjects. No INP104 related safety and adverse events were reported. There were no findings of concern from nasal endoscopy examinations or olfactory function assessments. INP104 safety and tolerability is summarized in Table 15, below:

TABLE 15
INP104 safety and tolerability

| Treatment-related treatment- | | | | |
|---|---|---|---|---|
| emergent adverse events | Full safety set | | Primary safety set | |
| (≥1% in full safety set) | N = 302 | | N = 103 | |
| Any IP related TEAE | 85 | (28.1%) | 30 | (29.1%) |
| Nasal congestion | 38 | (12.6%) | 13 | (12.6%) |
| Nausea | 16 | (5.3%) | 2 | (1.9%) |
| Nasal discomfort | 12 | (4.0%) | 5 | (4.9%) |
| Dysgeusia | 9 | (3.0%) | 2 | (1.9%) |
| Vomiting | 6 | (2.0%) | 2 | (1.9%) |
| Dizziness | 5 | (1.7%) | 1 | (1.0%) |
| Rhinorrhea | 5 | (1.7%) | 1 | (1.0%) |
| Nasal mucosal disorder | 4 | (1.3%) | 1 | (1.0%) |
| Sinus congestion | 4 | (1.3%) | 1 | (1.0%) |

### 7. INCORPORATION BY REFERENCE

The disclosures of each and every patent, patent application, and publication cited herein are hereby incorporated herein by reference in their entirety.

### 8. EQUIVALENTS

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of reducing the frequency of migraine attacks in a subject who has frequent migraine headaches with or without aura, comprising:
administering a pharmaceutical composition comprising dihydroergotamine (DHE) or salt thereof via a respiratory tract of the subject on a repeat dose schedule,
wherein the schedule is a chronic intermittent schedule in which each of the repeated administrations is performed while the subject is experiencing a migraine headache.

2. The method of claim 1, wherein the step of administering is performed by intranasal administration.

3. The method of claim 2, wherein the intranasal administration is performed with a manually actuated, propellant driven, metered dose administration device.

4. The method of claim 1, wherein the step of administering is performed by pulmonary administration.

5. The method of claim 1, wherein the repeat dose schedule comprises administration of at least a first dose and a second dose of the pharmaceutical composition.

6. The method of claim 1, wherein the pharmaceutical composition is a liquid pharmaceutical composition.

7. The method of claim 1, wherein each of the doses is administered as two divided subdoses, optionally wherein the divided subdoses are administered into separate nostrils.

8. The method of claim 3, wherein the propellant is a hydrofluoroalkane propellant.

9. The method of claim 8, wherein the propellant is hydrofluoroalkane-134a.

10. The method of claim 9, wherein prior to a first actuation, a vial is nonintegral to the device and is configured to be attached thereto, optionally wherein the vial is configured to be threadably attachable to the device.

11. The method of claim 1, wherein each of the doses of the pharmaceutical composition comprises no more than 2.0 mg DHE or salt thereof.

12. The method of claim 7, wherein the liquid composition is administered as two divided subdoses in two sprays, wherein each of the two divided subdoses is 140-250 μL.

13. The method of claim 6, wherein the liquid composition comprises a salt of DHE.

14. The method of claim 13, wherein the liquid composition comprises DHE mesylate, optionally at a concentration of 2.5-7.5 mg/ml.

15. The method of claim 13, wherein the liquid composition further comprises caffeine, optionally at a concentration of 10 mg/ml.

16. The method of claim 13, wherein the liquid composition further comprises dextrose, optionally at a concentration of 50 mg/ml.

17. The method of claim 1, wherein the pharmaceutical composition comprises 4.0 mg/ml DHE mesylate, 10.0 mg/ml caffeine, and 50 mg/ml dextrose.

18. The method of claim 1, wherein the subject has at least three migraine attacks in the 4-week period immediately preceding administration of the first dose.

19. The method of claim 18, wherein the subject has fewer than 3 migraine headaches during the 4-week period immediately following administration of the second dose.

20. The method of claim 19, wherein the subject has fewer than 2 migraine headaches during the 4-week period immediately following administration of the second dose.

21. The method of claim 1, wherein the subject has no migraine headaches during the 4-week period immediately following administration of the second dose.

22. The method of claim 1, wherein the subject has fewer than 6, fewer than 4, or fewer than 2 migraine headaches during the 8-week period immediately following administration of the second dose.

23. The method of claim 1, wherein the subject has fewer than 12, fewer than 6, or fewer than 3 migraine headaches in the 12-week period immediately following administration of the second dose.

24. The method of claim 1, wherein the subject has fewer than 18, fewer than 12 or fewer than 4 migraine headaches during the 24-week period immediately following the repeated administrations.

25. The method of claim 1, wherein the frequency of migraine headaches is reduced by at least 50%, 60%, or 75% during the 4-week period immediately following administration of the second dose as compared to the frequency of migraine headaches during the 4 week-period immediately preceding administration of the first dose.

26. The method of claim 1, wherein administration of the first dose of the repeated administrations of the pharmaceutical composition reduce one or more symptoms selected from pain, nausea, phonophobia, and photophobia.

27. The method of claim 26, wherein reduction of the one or more symptoms occurs at 2 hours post administration.

28. The method of claim 1, wherein the subject has migraine that does not respond to triptan drugs.

29. The method of claim 1, wherein the repeat dose schedule lasts at least one month.

30. The method of claim 29, wherein the repeat dose schedule lasts at least two months, at least three months, at least four months, at least five months, or at least six months.
